(19) United States
(12) Patent Application Publication
(10) Pub. No.: US 2021/0277003 A1
(43) Pub. Date: Sep. 9, 2021

(54) OGA INHIBITOR COMPOUNDS

(71) Applicant: Janssen Pharmaceutica NV, Beerse (BE)

(72) Inventors: José Manuel Bartolomé-Nebreda, Toledo (ES); Andrés Avelino Trabanco-Suárez, Olias del Rey (ES); Carolina Martinez Lamenca, Beerse (BE); Joseph Elisabeth Leenaerts, Rijkevorsel (BE); Daniel Oehlrich, Geel (BE); Petrus Jacobus Johannes Antonius Buijnsters, Etten-Leur (NL); Adriana Ingrid Velter, Antwerpen (BE); Yves Emiel Maria Van Roosbroeck, Antwerpen (BE)

(21) Appl. No.: 17/253,505

(22) PCT Filed: Jun. 20, 2019
(86) PCT No.: PCT/EP2019/066394

§ 371 Date: Dec. 17, 2020

(30) Foreign Application Priority Data
Jun. 21, 2018 (EP) 18382456.4

(51) Int. Cl. C07D 471/04; C07D 519/00

(52) U.S. Cl. CPC C07D 471/04; C07D 519/00

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

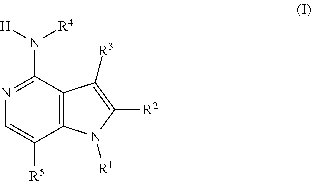

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA) O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in then-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensible for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying O-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyperphosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (AD) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2012/117219 (Summit Corp. plc., published 7 Sep. 2012) describes N-[[5-(hydroxymethyl)pyrrolidin-2-yl]methyl]alkylamide and N-alkyl-2-[5-(hydroxymethyl)pyrrolidin-2-yl]acetamide derivatives as OGA inhibitors; WO2016/0300443 (Asceneuron S. A., published 3 Mar. 2016), WO2017/144633 and WO2017/0114639 (Asceneuron S. A., published 31 Aug. 2017) disclose 1,4-disubstituted piperidines or piperazines as OGA inhibitors; WO2017/144637 (Asceneuron S. A, published 31 Aug. 2017) discloses more particular 4-substituted 1-[1-(1,3-benzodioxol-5-yl)ethyl]-piperazine; 1-[1-(2,3-dihydrobenzofuran-5-yl)ethyl]-; 1-[1-(2,3-dihydrobenzofuran-6-yl)ethyl]-; and 1-[1-(2,3-dihydro-1,4-benzodioxin-6-yl)ethyl]-piperazine derivatives as OGA inhibitors; WO2017/106254 (Merck Sharp & Dohme Corp.) describes substituted N-[5-[(4-methylene-1-piperidyl)methyl]thiazol-2-yl]acetamide compounds as OGA inhibitors.

There is still a need for OGA inhibitor compounds with an advantageous balance of properties, for example with improved potency, good bioavailability, pharmacokinetics, and brain penetration, and/or better toxicity profile. It is accordingly an object of the present invention to provide compounds that overcome at least some of these problems.

### SUMMARY OF THE INVENTION

The present invention is directed to compounds of Formula (I)

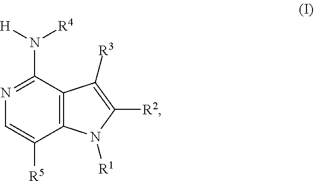

and the tautomers and the stereoisomeric forms thereof, wherein
R¹ is selected from the group consisting of C₁₋₆alkyl optionally substituted with one or more substituents each independently selected from the group consisting of halo, —CN, —OC₁₋₃alkyl, —OH, —SO₂NR^{5a}R^{6a}, and C₃₋₆cycloalkyl optionally substituted with one or more independently selected halo substituents; C₁₋₆alkyl substituted with oxetanyl; and C₁₋₆alkyl wherein two geminal hydrogens are replaced by oxetanylidene; wherein R^{5a} and R^{6a} are each independently selected from the group consisting of hydrogen and C₁₋₃alkyl; with the proviso that a —OC₁₋₃alkyl or —OH substituent, when present, is at least two carbon atoms away from the nitrogen atom of the 1H-pyrrolo[3.2-c]pyridine;
R², R³ and R⁵ are each independently selected from the group consisting of hydrogen, halo and C₁₋₃alkyl;
R⁴ is a monovalent radical selected from the group consisting of (a), (b), (c), and (d):

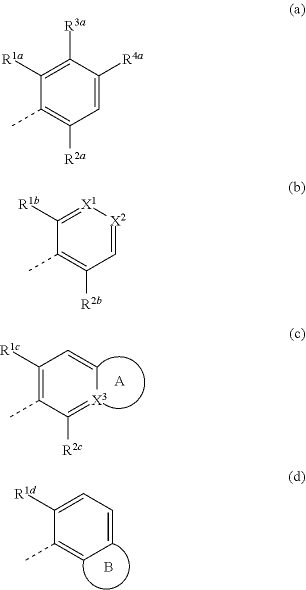

wherein
R^{1a}, R^{2a}, R^{1b}, and R^{2b} are each independently selected from the group consisting of halo, C₁₋₃alkyl, monohaloC₁₋₃alkyl, polyhaloC₁₋₃alkyl, C₁₋₃alkyloxy, monohaloC₁₋₃alkyloxy, polyhaloC₁₋₃alkyloxy, and C₃₋₆cycloalkyl;
R^{3a} is selected from the group consisting of hydrogen, halo, —C(O)—OC₁₋₃alkyl, —C(O)—NR′R″, and —N(R′″)—C(O)—C₁₋₃alkyl;
R^{4a} is selected from the group consisting of hydrogen, halo, —CN, C₁₋₃alkyl, monohaloC₁₋₃alkyl, polyhaloC₁₋₃alkyl, —C(O)—OC₁₋₃alkyl, —C(O)—NR′R″, —N(R′″)—C(O)—C₁₋₃alkyl, and Het;
with the proviso that R^{3a} and R^{4a} are not simultaneously —C(O)—OC₁₋₃alkyl, —C(O)—NR′R″, or —N(R′″)—C(O)—C₁₋₃alkyl;
R′ and R″ are each independently selected from the group consisting of hydrogen and C₁₋₃alkyl; or R′ and R″ together with the nitrogen atom to which they are attached form a heterocyclyl ring selected from the group consisting of azetidinyl, pyrrolidinyl, piperidinyl, piperazinyl and morpholinyl;
R′″ is selected from the group consisting of hydrogen and C₁₋₃alkyl;
Het is pyrazolyl or imidazolyl, optionally substituted with one or more independently selected C₁₋₃alkyl substituents;
X¹ and X² are each independently selected from N and CH, with the proviso that at least one of X¹ or X² is N;
R^{1c}, R^{2c}, and R^{1d} are each independently selected from the group consisting of halo, C₁₋₃alkyl, monohaloC₁₋₃alkyl, polyhaloC₁₋₃alkyl, C₁₋₃alkyloxy, monohaloC₁₋₃alkyloxy, polyhaloC₁₋₃alkyloxy, and C₃₋₆cycloalkyl;
X³ represents CH or N;
and each of the rings represented by

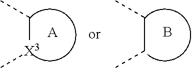

form
(i) a 5- or 6-membered unsaturated heterocycle having one, two or three heteroatoms each independently selected from nitrogen and oxygen, and which is optionally substituted with one or more substituents, each independently selected from halo, C₁₋₃alkyl and oxo; or
(ii) an aromatic heterocycle having one, two or three heteroatoms each independently selected from nitrogen, oxygen, and sulfur, and which is optionally substituted with one or more substituents, each independently selected from halo, —CN, C₁₋₃alkyl, monohaloC₁₋₃alkyl, and polyhaloC₁₋₃alkyl;
and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a therapeutically effective amount of any of the compounds or pharmaceutical compositions described above.

Further exemplifying the invention are methods of inhibiting OGA, comprising administering to a subject in need thereof a prophylactically or a therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I), as defined herein before, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or may be useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In a particular embodiment, the invention is directed to compounds of Formula (I) as defined hereinbefore, and the tautomers and the stereoisomeric forms thereof, wherein R¹ is selected from the group consisting of C₁₋₆alkyl optionally substituted with one, two or three substituents each independently selected from the group consisting of halo, —CN, —OC₁₋₃alkyl, —OH, —SO₂NR^{5a}R^{6a}, and C₃₋₆cycloalkyl optionally substituted with one, two or three independently selected halo substituents; C₁₋₆alkyl substituted with oxetanyl; and C₁₋₆alkyl wherein two geminal hydrogens are replaced by oxetanylidene; wherein R^{5a} and R^{6a} are each independently selected from the group consisting of hydrogen and C₁₋₃alkyl; with the proviso that a —OC₁₋₃alkyl or —OH substituent, when present, is at least two carbon atoms away from the nitrogen atom of the 1H-pyrrolo[3.2-c]pyridine;

R², R³ and R⁵ are each independently selected from the group consisting of hydrogen, halo and C₁₋₃alkyl;
R⁴ is a monovalent radical selected from the group consisting of (a), (b), (c), and (d), wherein
R^{1a}, R^{2a}, R^{1b}, and R^{2b} are each independently selected from the group consisting of halo, C₁₋₃alkyl, monohaloC₁₋₃alkyl, polyhaloC₁₋₃alkyl, and C₃₋₆cycloalkyl;
R^{3a} is selected from the group consisting of hydrogen, halo, —C(O)—NR′R″, and —N(R′″)—C(O)—C₁₋₃alkyl;
R^{4a} is selected from the group consisting of hydrogen, halo, C₁₋₃alkyl, monohaloC₁₋₃alkyl, polyhaloC₁₋₃alkyl, —C(O)—OC₁₋₃alkyl, —C(O)—NR′R″, —N(R′″)—C(O)—C₁₋₃alkyl, and Het; with the proviso that R^{3a} and R^{4a} are not simultaneously —C(O)—OC₁₋₃alkyl, —C(O)—NR′R″, or —N(R′″)—C(O)—C₁₋₃alkyl; R′ and R″ are each independently selected from the group consisting of hydrogen and C₁₋₃alkyl; or R′ and R″ together with the nitrogen atom to which they are attached form a heterocyclyl ring selected from the group consisting of azetidinyl, pyrrolidinyl, piperidinyl, piperazinyl and morpholinyl;
R′″ is selected from the group consisting of hydrogen and C₁₋₃alkyl;
Het is pyrazolyl or imidazolyl, optionally substituted with one or more independently selected C₁₋₃alkyl substituents;
X¹ and X² are each independently selected from N and CH, with the proviso that at least one of X¹ or X² is N;
R^{1c}, R^{2c}, and R^{1d} each independently represent halo or C₁₋₃alkyl;
X³ represents CH or N;
and each of the rings represented by

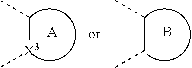

form
(i) a 5- or 6-membered unsaturated heterocycle having one, two or three heteroatoms each independently selected from nitrogen and oxygen, and which is optionally substituted with one or two substituents, each independently selected from halo, C₁₋₃alkyl and oxo; or
(ii) an aromatic heterocycle having one, two or three heteroatoms each independently selected from nitrogen and oxygen, and which is optionally substituted with one or two substituents, each independently selected from C₁₋₃alkyl;
and the pharmaceutically acceptable salts and the solvates thereof.

In a particular embodiment, the invention is directed to compounds of Formula (I) as referred to herein, and the tautomers and the stereoisomeric forms thereof, wherein R¹ is selected from the group consisting of C₁₋₆alkyl optionally substituted with one, two or three substituents each independently selected from the group consisting of halo, and C₃₋₆cycloalkyl optionally substituted with one, two or three independently selected halo substituents; C₁₋₆alkyl substituted with oxetanyl; and C₁₋₆alkyl wherein two geminal hydrogens are replaced by oxetanylidene;

R², R³ and R⁵ are each independently selected from the group consisting of hydrogen, halo and C₁₋₃alkyl;
R⁴ is a monovalent radical selected from the group consisting of (a), (b), (c), and (d), wherein
R^{1a}, R^{2a}, R^{1b}, and R^{2b} are each independently selected from the group consisting of halo, C₁₋₃alkyl, monohaloC₁₋₃alkyl, polyhaloC₁₋₃alkyl, and C₃₋₆cycloalkyl;
R^{3a} is selected from the group consisting of hydrogen, halo, and —C(O)—NR′R″;
R^{4a} is selected from the group consisting of hydrogen, halo, C₁₋₃alkyl, monohaloC₁₋₃alkyl, polyhaloC₁₋₃alkyl, —C(O)—OC₁₋₃alkyl, —C(O)—NR′R″, —N(R′″)—C(O)—C₁₋₃alkyl, and Het;
with the proviso that R^{3a} and R^{4a} are not simultaneously —C(O)—OC₁₋₃alkyl, —C(O)—NR′R″, or —N(R′″)—C(O)—C₁₋₃alkyl;
R′ and R″ are each independently selected from the group consisting of hydrogen and C₁₋₃alkyl; or R′ and R″ together with the nitrogen atom to which they are attached form a heterocyclyl ring selected from the group consisting of pyrrolidinyl, and morpholinyl;
R′″ is selected from the group consisting of hydrogen and C₁₋₃alkyl;
Het is pyrazolyl or imidazolyl, optionally substituted with one or more independently selected C₁₋₃alkyl substituents;
X¹ and X² are each independently selected from N and CH, with the proviso that at least one of X¹ or X² is N;
R^{1c}, R^{2c}, and R^{1d} each independently represent halo or C₁₋₃alkyl;
X³ represents CH or N;
and each of the rings represented by

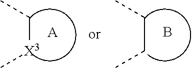

form
(i) a 5- or 6-membered unsaturated heterocycle having one, two or three heteroatoms each independently selected from nitrogen and oxygen, and which is optionally substituted with one or two substituents, each independently selected from halo, C₁₋₃alkyl and oxo; or
(ii) an aromatic heterocycle having one, two or three heteroatoms each independently selected from nitrogen and oxygen, and which is optionally substituted with one or two substituents, each independently selected from C₁₋₃alkyl;
and the pharmaceutically acceptable salts and the solvates thereof.

In a further embodiment, the invention is directed to compounds of Formula (I), as referred to herein, wherein R¹ is C₁₋₆alkyl optionally substituted with one, two or three substituents each independently selected from the group consisting of halo, and C₃₋₆cycloalkyl optionally substituted with one, two or three independently selected halo substituents or R¹ is C₁₋₆alkyl substituted with oxetanyl or C₁₋₆alkyl wherein two geminal hydrogens are replaced by oxetanylidene.

In a particular embodiment, the invention is directed to compounds of Formula (I), as referred to herein, wherein R¹ is C₁₋₆alkyl optionally substituted with one, two or three substituents each independently selected from the group consisting of halo, and C₃₋₆cycloalkyl optionally substituted with one, two or three independently selected halo substituents.

In an additional embodiment, the invention is directed to compounds of Formula (I) as referred to herein, wherein R¹ is C₁₋₆alkyl substituted with oxetanyl or C₁₋₆alkyl wherein two geminal hydrogens are replaced by oxetanylidene.

In an additional embodiment, the invention is directed to compounds of Formula (I) as referred to herein wherein R¹ is

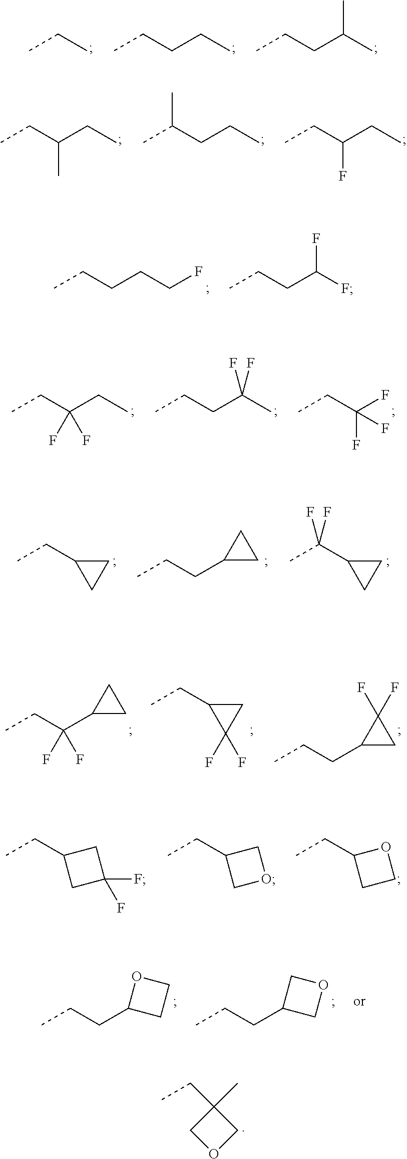

In an additional embodiment, the invention is directed to compounds of Formula (I) as referred to herein, wherein R¹ is

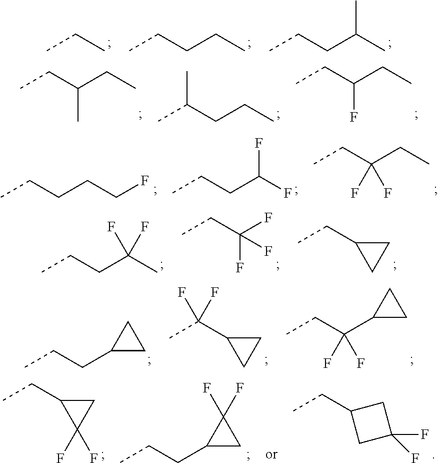

In an additional embodiment, the invention is directed to compounds of Formula (I) as referred to herein, wherein R¹ is

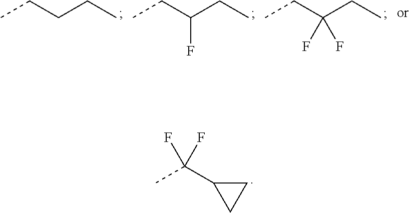

In an additional embodiment, the invention is directed to compounds of Formula (I) as referred to herein, wherein R¹ is

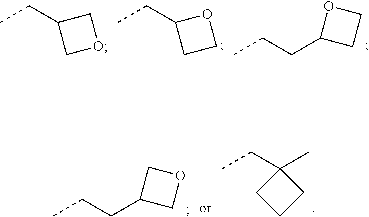

In a further embodiment, the invention is directed to compounds of Formula (I), as referred to herein, and the tautomers and the stereoisomeric forms thereof, wherein

R⁴ is a monovalent radical selected from the group consisting of (a), (b), and (c), wherein R^{1a}, R^{2a}, R^{1b}, and R^{2b} are each independently selected from the group consisting of halo and C₁₋₃alkyl;
R^{3a} is hydrogen;
R^{4a} is selected from the group consisting of hydrogen, —C(O)—NR′R″, and —N(R′″)—C(O)—C₁₋₃alkyl;
R′ and R″ are each independently selected from the group consisting of hydrogen and C₁₋₃alkyl; or R′ and R″ together with the nitrogen atom to which they are attached form a heterocyclyl ring selected from the group consisting of pyrrolidinyl, and morpholinyl;
R′″ is hydrogen;
X¹ is N and X² is CH;
R^{1c} and R^{2c} each independently represent halo or C₁₋₃alkyl;
X³ represents CH;
and

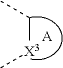

forms an imidazole optionally substituted with one or two independently selected C₁₋₃alkyl substituents;
and the pharmaceutically acceptable salts and the solvates thereof.

In another embodiment, the invention is directed to compounds of Formula (I), as referred to herein, wherein R² and R³ are each independently selected from hydrogen and fluoro.

In a further embodiment, the invention is directed to compounds of Formula (I), as referred to herein, wherein R⁵ is hydrogen, fluoro or methyl.

### Definitions

“Halo” shall denote fluoro, chloro and bromo, in particular fluoro or chloro; “oxo” shall denote ═O, i.e. an oxygen atom doubly bound to a carbon atom; “C₁₋₃alkyl” shall denote a straight or branched saturated alkyl group having 1, 2, or 3 carbon atoms, respectively, e.g. methyl, ethyl, 1-propyl, 2-propyl; “C₁₋₆alkyl” shall denote a straight or branched saturated alkyl group having 1, 2, 3, 4, 5 or 6 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₃alkyloxy” shall denote an ether radical wherein C₁₋₃alkyl is as defined before; “monohalo-C₁₋₃alkyl, polyhalo-C₁₋₃alkyl” as used herein alone or as part of another group, shall denote a C₁₋₃alkyl as defined before, substituted with 1, 2, 3 or where possible with more halo atoms as defined before; “C₃₋₆cycloalkyl” as used herein shall denote a saturated, cyclic hydrocarbon radical having from 3 to 6 carbon atoms, such as cyclopropyl, cyclobutyl, cyclopentyl, and cyclohexyl. A particular C₃₋₆cycloalkyl group is cyclopropyl.

Examples of a 5- or 6-membered unsaturated heterocycle having one, two or three heteroatoms each independently selected from nitrogen, oxygen, and sulfur, and which is optionally substituted with one or two substituents, each independently selected from halo, C₁₋₃alkyl and oxo, include, but are not limited to tetrahydrofurane, tetrahydropyrane, 1,4-dioxane, pyrrolidine, piperidine, piperazine, morpholine, lactam (e.g. pyrrolidinone, piperidinone), and the like.

Examples of an aromatic heterocycle having one, two or three heteroatoms each independently selected from nitrogen and oxygen, and which is optionally substituted with one or two substituents, each independently selected from C₁₋₃alkyl, includes, but are not limited to pyrrole, pyrazole, imidazole, triazole, and the like.

Whenever the term “substituted” is used in the present invention, it is meant, unless otherwise is indicated or is clear from the context, to indicate that one or more hydrogens, preferably from 1 to 3 hydrogens, more preferably from 1 to 2 hydrogens, more preferably 1 hydrogen, on the atom or radical indicated in the expression using “substituted” are replaced with a selection from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Experimental Procedure 1

Final compounds of Formula (I) can be prepared by reacting an intermediate compound of Formula (II-a) with a compound of Formula (III) according to reaction scheme 1. The reaction is performed in a suitable reaction-inert solvent, such as for example tBuOH, in the presence of a base, such as Cs₂CO₃ or K₃PO₄, in the presence of a catalyst, such as Pd(OAc)₂ or Pd₂dba₃, and a suitable phosphorus ligand, such as XantPhos, under thermal conditions, such as for example at 110-130° C. for a suitable period of time to drive the reaction to completion. In reaction scheme 1 all variables are defined as in Formula (I) and halo represents a halogen, in particular, bromo or chloro.

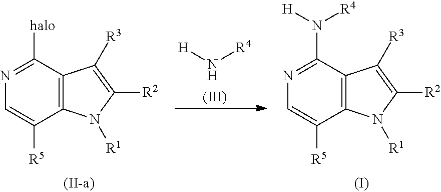

### Experimental Procedure 2

Alternatively, final compounds of Formula (I) can be prepared by reacting an intermediate compound of Formula (II-b) with a compound of Formula (IV) according to reaction scheme 2. The reaction is performed under the same conditions as described in experimental procedure 1.

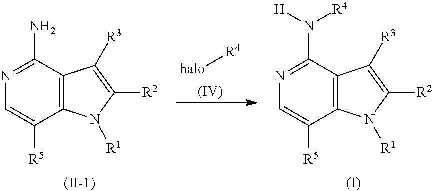

### Experimental Procedure 3

Alternatively, final compounds of Formula (I) can be prepared by reacting an intermediate compound of Formula (II-c) with a compound of Formula (V) according to reaction scheme 3. The reaction is performed in a suitable reaction-inert solvent, such as for example DMF, in the presence of a suitable base such as for example NaH, at a suitable temperature, such as for example 0° C. to room temperature for a suitable period of time to drive the reaction to completion. In reaction scheme 3 all variables are defined as in Formula (I) and halo represents a halogen, in particular, bromo or chloro.

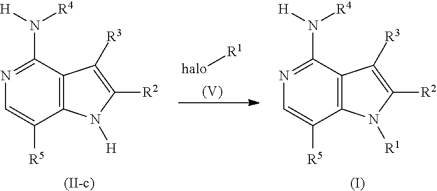

### Experimental Procedure 4

Intermediate compounds of Formula (II-a) wherein R² is fluoro, herein referred to as (II-a1), can be prepared by reacting an intermediate compound of Formula (VI) with N-fluorobenzenesulfonimide under reaction conditions known to the skilled person, such as for example, in THF at −78° C. to RT to the preformed carbanion, according to reaction scheme 4. In reaction scheme 4 all variables are defined as in Formula (I) and halo represents a halogen, in particular, bromo or chloro.

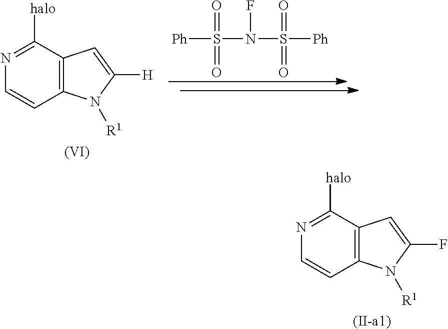

### Experimental Procedure 5

Intermediate compounds of Formula (II-a) wherein R³ is fluoro, herein referred to as (II-a2), can be prepared by reacting an intermediate compound of Formula (VII) with SelectFluor® under reaction conditions known to the skilled person, such as for example, in nitroethane at 0° C., according to reaction scheme 5. In reaction scheme 5 all variables are defined as in Formula (I) and halo represents a halogen, in particular, bromo or chloro.

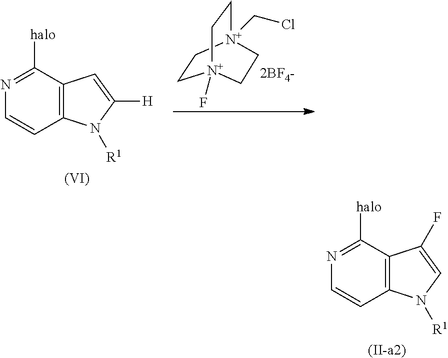

Intermediate compounds of Formulae (II-a), (II-b), (II-c) and (VI) are either commercially available or can be synthesized according to reaction procedures known to the skilled person.

### Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical states in Alzheimer's and tauopathy diseases: In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that Aβ accumulation and tau-aggregation begins many years before the onset of overt clinical impairment. The key risk factors for elevated amyloid accumulation, tau-aggregation and development of AD are age (ie, 65 years or older), APOE genotype, and family history. Approximately one third of clinically normal older individuals over 75 years of age demonstrate evidence of Aβ or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

### Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The invention also provides a kit comprising a compound according to the invention, prescribing information also known as “leaflet”, a blister package or bottle, and a container. Furthermore, the invention provides a kit comprising a pharmaceutical composition according to the invention, prescribing information also known as “leaflet”, a blister package or bottle, and a container. The prescribing information preferably includes advice or instructions to a patient regarding the administration of the compound or the pharmaceutical composition according to the invention. In particular, the prescribing information includes advice or instruction to a patient regarding the administration of said compound or pharmaceutical composition according to the invention, on how the compound or the pharmaceutical composition according to the invention is to be used, for the prevention and/or treatment of a tauopathy in a subject in need thereof. Thus, in an embodiment, the invention provides a kit of parts comprising a compound of Formula (I) or a stereoisomeric for thereof, or a pharmaceutically acceptable salt or a solvate thereof, or a pharmaceutical composition comprising said compound, and instructions for preventing or treating a tauopathy. The kit referred to herein can be, in particular, a pharmaceutical package suitable for commercial sale.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### EXPERIMENTAL PART

Hereinafter, the term “AcOH” means acetic acid, “aq.” means aqueous, “Boc” means tert-butoxycarbonyl, “DAST” means (diethylamino)sulfur trifluoride, “DCE” means dichloroethane, “DCM” means dichloromethane, “DMF” means dimethylformamide, “DIBAL” means diisobutylaluminium hydride, “DIPE” means diisopropyl ether, “DME” means dimethylether, “DIPA” means diisopropylamine, “DMSO” means dimethyl sulfoxide, “EtOAc” means ethyl acetate, “EtOH” means ethanol, “Et₃N” means triethylamine, “Et₂O” means diethyl ether, “HATU” means N-[(dimethylamino)-1H-1,2,3-triazolo-[4,5-b]pyridin-1-ylmethylene]-N-methylmethanaminium hexafluorophosphate N-oxide, “HPLC” means high-performance liquid chromatography, “i-PrNH₂” means isopropylamine, “i-PrOH” means isopropyl alcohol, “LC-MS” means liquid chromatography/mass spectrometry, “LiHMDS” means lithium bis(trimethylsilyl)amide, “MeOH” means methanol, “[M+H]+” means the protonated mass of the free base of the compound, “MIK” means methyl isobutyl ketone, “m.p.” means melting point, “min” means minutes, “MW” means microwave, “NP” means normal phase, “ol” or “OL” means organic layer, “org.” means organic, “Pd/C” means palladium on carbon, “Pd(OAc)₂” means palladium(II) acetate, “Pd₂dba₃” means tris(dibenzylideneacetone)dipalladium(0), “Pd(dppf)Cl₂” means [1,1′-bis(diphenylphosphino)ferrocene]dichloropalladium(II), “Pd(PPh₃)₃” means tetrakis(triphenylphosphine)palladium(0), “r.m.” means reaction mixture, “RP” means reversed phase, “Rt” means retention time (in minutes), “r.t.” or “RT” means room temperature, “rac” or “RS” means racemic, “sat.” means saturated, “SFC” means supercritical fluid chromatography, “SFC-MS” means supercritical fluid chromatography/mass spectrometry, SelectFluor® means 1-chloromethyl-4-fluoro-1,4-diazoniabicyclo[2.2.2]octane bis(tetrafluoroborate), “sol.” means solution, “TBAF” means tetrabutylammonium fluoride hydrate, “TFA” means trifluoroacetic acid, “THF” means tetrahydrofuran, “TLC” means thin layer chromatography, “t-BuOH” means tert-butanol, “wt” means weight, “XantPhos” means 4,5-bis(diphenylphosphino)-9,9-dimethylxanthene, “XPhos” means 2-dicyclohexylphosphino-2′,4′,6′-triisopropylbiphenyl.

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “R*” or “S*” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques.

Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or PuriFlash® 430evo systems from Interchim, or 971-FP systems from Agilent, or Isolera ISV systems from Biotage.

### Preparation of Intermediate Compounds

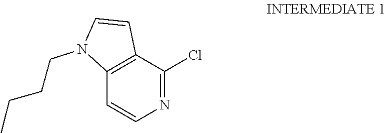

To a solution of 4-chloro-1H-pyrrolo-[3,2-c]-pyridine [60290-21-3] (2.0 g, 13.1 mmol) dissolved in DMF (30.5 mL, 0.944 g/mL, 393.2 mmol) at 0° C. was added portionwise sodium hydride (1.1 g, 28.8 mmol). The reaction mixture was allowed to reach rt and stirred 45 min, after which it was re-cooled to 0° C. and 1-bromobutane (2.1 mL, 1.27 g/mL, 19.7 mmol) was added dropwise. The mixture was then allowed to reach rt and stirred overnight. NaHCO₃ sat solution was added and the aqueous phase was extracted with EtOAc. The combined organic extracts were washed with water and brine, then dried over MgSO₄ and concentrated in vacuo. The crude residue was purified by column chromatography (silica gel; gradient Heptane/EtOAc from 100/0 to 50/50) to yield I-1 (2.7 g, 98.7%) as a yellow liquid.

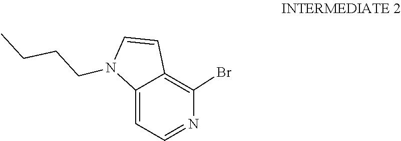

I-2 was prepared in a similar manner to I-1, starting from 4-bromo-1H-pyrrolo[3,2-c]pyridine [1000342-68-6] (2 g, 10.2 mmol) and 1-bromobutane (1.65 mL, 15.2 mmol) to yield I-2 (2.33 g, 91%) as a yellow liquid.

The following intermediates were prepared in an analogous manner from the indicated starting material, either starting with 4-bromo-1H-pyrrolo[3,2-c]pyridine ([1000342-68-6]) or 4-chloro-1H-pyrrolo[3,2-c]pyridine ([60290-21-3]).

| STARTING | | |
|---|---|---|
| MATERIAL | REAGENT | INTERMEDIATE |
| [60290-21-3] | 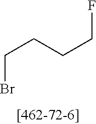 | 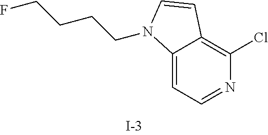 [SMILES: FCCCCn1ccc2c(Cl)nccc21] |
| [60290-21-3] | 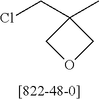 | 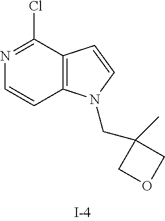 [SMILES: CC1(Cn2ccc3c(Cl)nccc32)COC1] |
| ([60290-21-3]) = Cl ([1000342-68-6]) = Br | 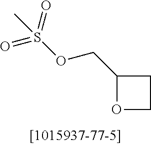 | 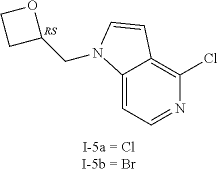 |
| ([60290-21-3]) = Cl ([1000342-68-6]) = Br | 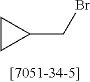 | 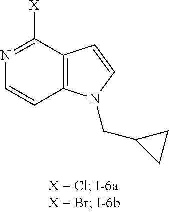 |
| ([60290-21-3]) = Cl ([1000342-68-6]) = Br | 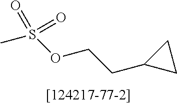 | 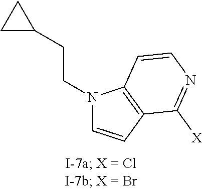 |
| [60290-21-3] | 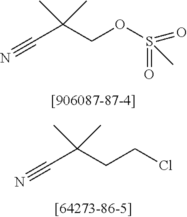 | 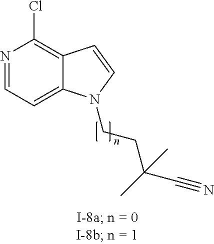 |
| [60290-21-3] | 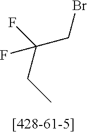 | 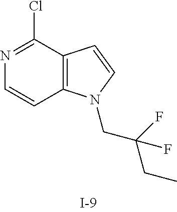 [SMILES: CCC(F)(F)Cn1ccc2c(Cl)nccc21] |
| [60290-21-3] | 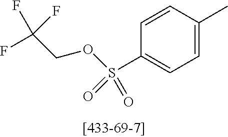 [SMILES: Cc1ccc(S(=O)(=O)OCC(F)(F)F)cc1] | 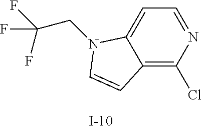 [SMILES: FC(F)(F)Cn1ccc2c(Cl)nccc21] |
| [60290-21-3] | 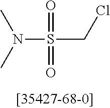 | 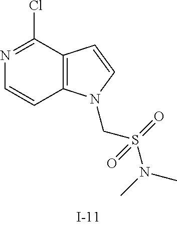 [SMILES: CN(C)S(=O)(=O)Cn1ccc2c(Cl)nccc21] |
| [60290-21-3] | 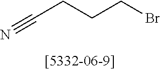 | 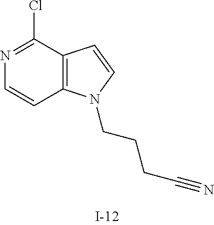 [SMILES: N#CCCCn1ccc2c(Cl)nccc21] |
| [60290-21-3] | 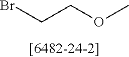 | 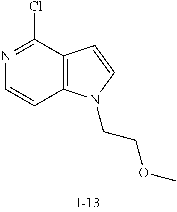 |
| [60290-21-3] | 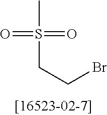 | 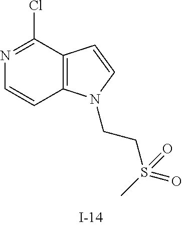 [SMILES: CS(=O)(=O)CCn1ccc2c(Cl)nccc21] |
| [60290-21-3] | 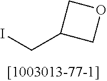 | 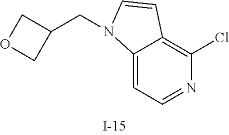 [SMILES: Clc1nccc2c1ccn2CC1COC1] |
| [60290-21-3] | 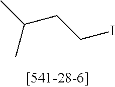 | 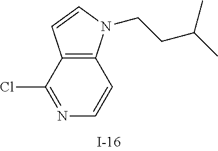 |
| [60290-21-3] | 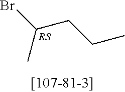 | 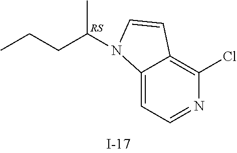 |
| ([60290-21-3]) = Cl ([1000342-68-6]) = Br | 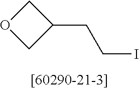 | 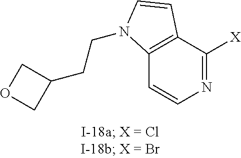 |
| [60290-21-3] | 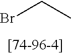 | 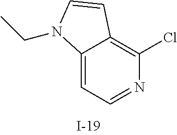 |
| [60290-21-3] | 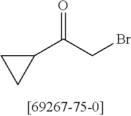 | 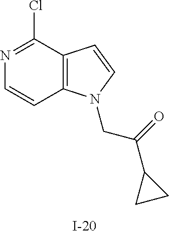 [SMILES: O=C(Cn1ccc2c(Cl)nccc21)C1CC1] |
| [60290-21-3] | 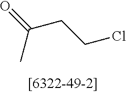 | 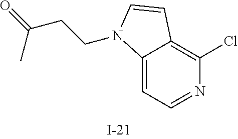 |
| [60290-21-3] | 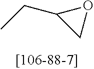 | 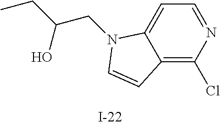 |
| [1000342-68-6] | 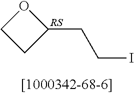 | 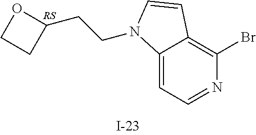 |
| [60290-21-3] | 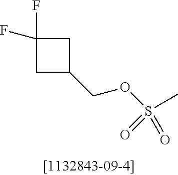 | 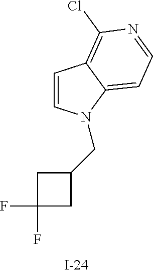 [SMILES: FC1(F)CC(Cn2ccc3c(Cl)nccc32)C1] |
| [60290-21-3] | 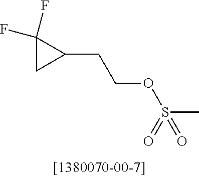 | 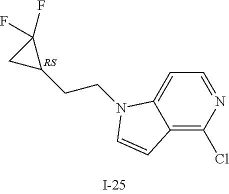 |
| 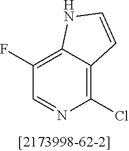 [SMILES: Fc1cnc(Cl)c2cc[nH]c12] | 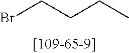 | 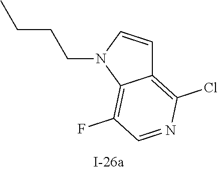 |
| 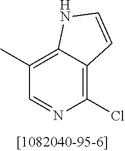 | 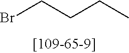 | 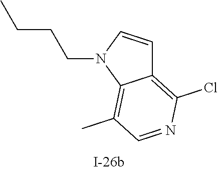 |
| [1000342-68-6] | 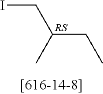 | 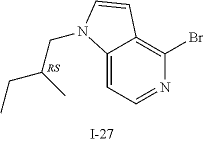 |
| 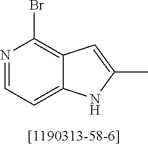 | 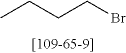 | 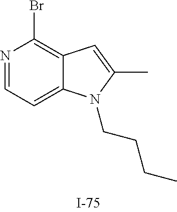 |
| 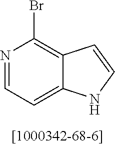 | 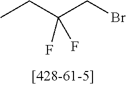 | 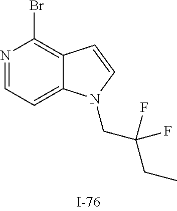 [SMILES: CCC(F)(F)Cn1ccc2c(Br)nccc21] |
| 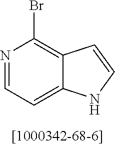 | 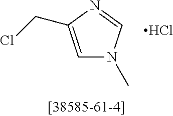 | 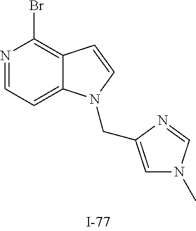 [SMILES: Cn1cnc(Cn2ccc3c(Br)nccc32)c1] |
| 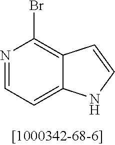 | 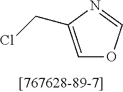 | 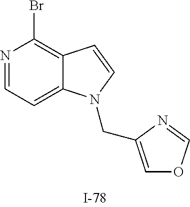 [SMILES: Brc1nccc2c1ccn2Cc1cocn1] |
| 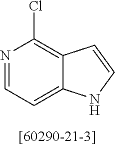 | 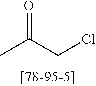 | 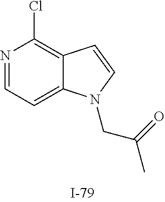 |
| 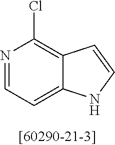 | 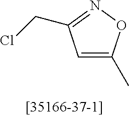 | 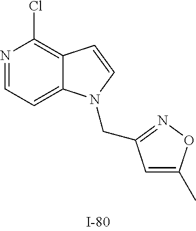 [SMILES: Cc1cc(Cn2ccc3c(Cl)nccc32)no1] |
| 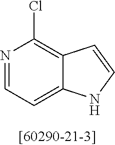 | 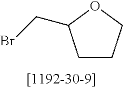 | 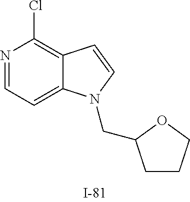 [SMILES: Clc1nccc2c1ccn2CC1CCCO1] |
| 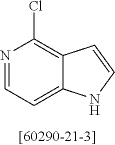 | 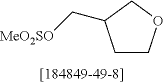 | 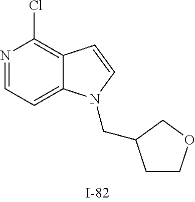 [SMILES: Clc1nccc2c1ccn2CC1CCOC1] |
| 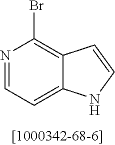 | 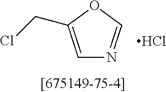 | 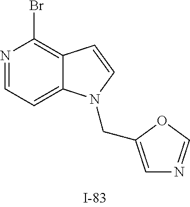 [SMILES: Brc1nccc2c1ccn2Cc1cnco1] |
| 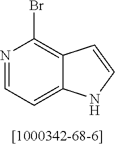 | 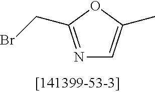 | 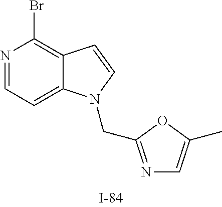 [SMILES: Cc1cnc(Cn2ccc3c(Br)nccc32)o1] |
| 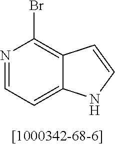 | 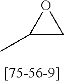 | 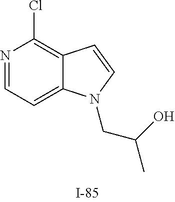 |
| 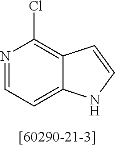 | 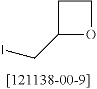 | 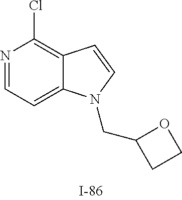 [SMILES: Clc1nccc2c1ccn2CC1CCO1] |
| 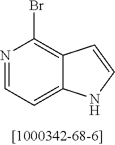 | 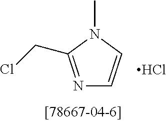 | 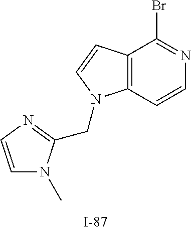 [SMILES: Cn1ccnc1Cn1ccc2c(Br)nccc21] |
| 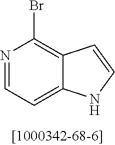 | 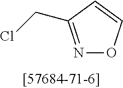 | 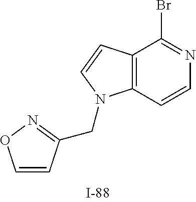 [SMILES: Brc1nccc2c1ccn2Cc1ccon1] |
| 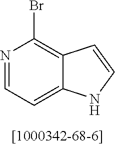 | 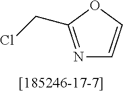 | 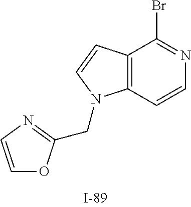 [SMILES: Brc1nccc2c1ccn2Cc1ncco1] |
| 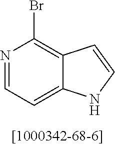 | 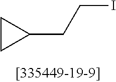 | 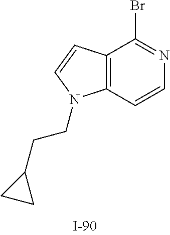 [SMILES: Brc1nccc2c1ccn2CCC1CC1] |
| 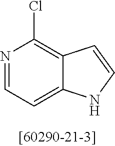 | 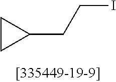 | 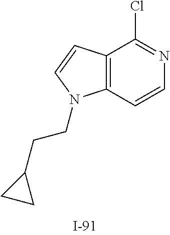 [SMILES: Clc1nccc2c1ccn2CCC1CC1] |

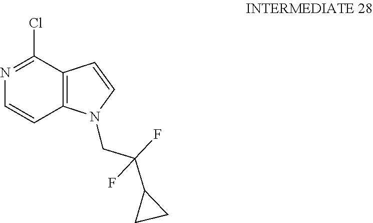 [SMILES: FC(F)(Cn1ccc2c(Cl)nccc21)C1CC1]

A solution of DAST [38078-09-0] (1.04 mL, 8.49 mmol) was added dropwise to a solution of I-20 (465 mg, 1.98 mmol) in dry DCM, (42.46 mL). The resulting solution was stirred at 35° C. for 48 h, after which the reaction was quenched by the addition of a sat. sol. of sodium bicarbonate. The RM was then extracted three times using DCM. The OL was dried over Na₂SO₄, filtered and concentrated in vacuo. The crude residue was purified by column chromatography (silica gel, EtOAc in Heptane, gradient from 0 to 30%). The pure fractions were evaporated, yielding I-28 (164 mg, 32%) as a sticky solid.

The following intermediates were synthesized in an analogous manner, from the indicated starting materials:

| STARTING MATERIAL | INTERMEDIATE |
|---|---|
| 1-20 | 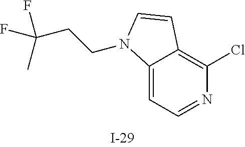 [SMILES: CC(F)(F)CCn1ccc2c(Cl)nccc21] |
| 1-22 | 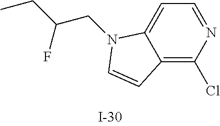 |
| 1-79 | 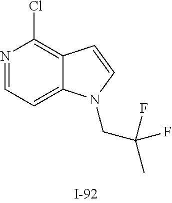 |

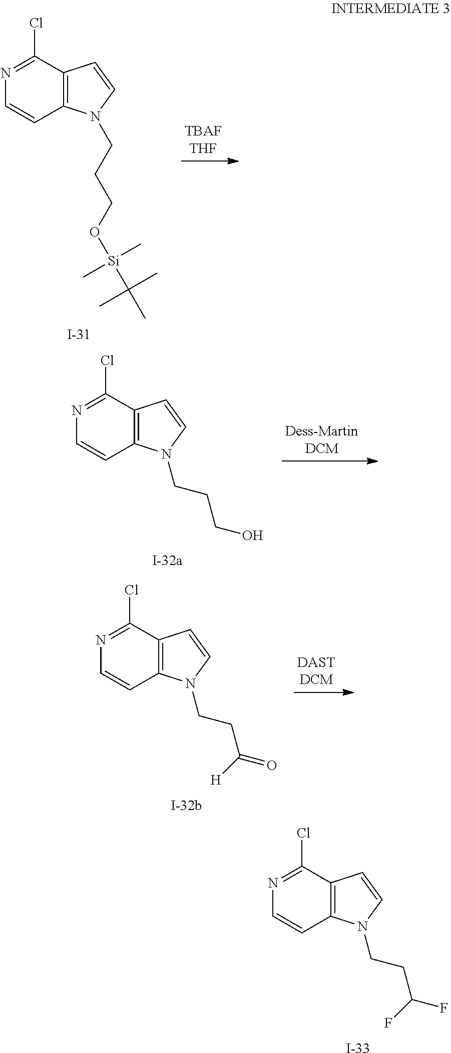

To a solution of 4-chloro-1H-pyrrolo-[3,2-c]-pyridine [60290-21-3] (1.0 g, 6.5 mmol) dissolved in DMF (51 mL) at 0° C. was added portionwise sodium hydride (288 mg, 7.2 mmol). The reaction mixture was allowed to reach rt and stirred 45 min, after which it was re-cooled to 0° C. and (3-bromopropoxy)-tert-butyldimethylsilane [89031-84-5] (2.5 g, 9.8 mmol) was added dropwise. The mixture was then allowed to reach rt and stirred overnight. NaHCO₃ sat solution was added and the aqueous phase was extracted with EtOAc The combined organic extracts were washed with water and brine, then dried over MgSO₄ and concentrated in vacuo to afford. The residue was purified by column chromatography (silica gel; DCM/MeOH, gradient from 100/0 to 95/5)) to yield I-31 (2.7 g, 98.7%) as a yellow liquid.

I-31 (1.67 g, 5.146 mmol) was dissolved in THF (41 mL) and TBAF (1M in THF, 6.7 mL, 6.69 mmol) was added and the rm was stirred at room temp for 1 h. The RM was concentrated in vacuo and the residue was partitioned between an aq. sol. of NaHCO₃ and DCM, and extracted with DCM. The organic fraction was dried over MgSO₄ and concentrated in vacuo. The residue was purified by column chromatography (silica gel; DCM/MeOH, gradient from 100/0 to 95/5) to yield I-32a (1 g, 92%).

To a solution of I-32a (900 mg, 4.272 mmol) in DCM (21 mL) was added Dess-Martin periodinane (1.9 g, 4.486 mmol) in one portion at 0° C. The reaction mixture was stirred at rt for 1 h. The reaction mixture was quenched with sat. aq. NaHCO₃ and sat. aq. Na₂S₂O₃ was added and the reaction mixture stirred for 30 min. The organic layer was separated, washed with brine, dried over MgSO₄ and the solvent was removed under vacuum to afford I-32b which was used in next step without purification (900 mg, yield 100%).

I-32b (891.34 mg, 4.3 mmol) was suspended in DCM (178 mL) and cooled down to 0° C. Diethylaminosulfur trifluoride (1 mL, 4.3 mmol) was added dropwise. Then the reaction mixture was stirred first at 0° C. and then allowed to warm to rt. After 3 h at rt, the reaction mixture was treated with water and NaHCO₃ and extracted with DCM. The combined extracts were washed with water, dried over MgSO₄, filtered and concentrated. The crude residue was purified by column chromatography (silica gel; eluent: DCM) to afford I-33 (425 mg, yield 43%).

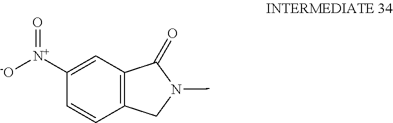

A solution of methyl 2-(bromomethyl)-5-nitro-benzoate [90725-68-1] (1 g, 3.65 mmol) and methylamine (40% in water, 0.346 mL, 4.014 mmol) in MeOH (8 mL) was stirred rt for 16 h. Water was added and the mixture was extracted with EtOAc. The combined organic layers were dried over MgSO₄, filtered and evaporated in vacuo to yield I-34 (700 mg, quantitative) as yellow solid.

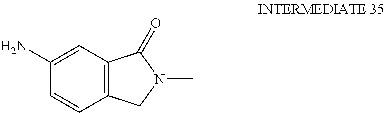

Pd/C (10%, 96.911 mg, 0.0911 mmol) was added to a stirred solution of I-34 (700 mg, 3.64 mmol) in MeOH (8 mL) and EtOH (8 mL) under nitrogen atmosphere. The mixture was hydrogenated H2 (atmospheric pressure) at rt for 18 h. The mixture was filtered through a pad of diatomaceous earth and the residue was washed with MeOH. The filtrate was evaporated in vacuo to yield I-35 (590.78 mg, quantitative) as a yellow solid.

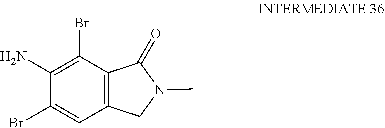

I-35 (0.591 g, 3.643 mmol) was dissolved in acetic acid (7.5 mL) and CHCl₃ (7.5 mL). Then a solution of Br₂ (0.411 mL, 8.01 mmol) in acetic acid (2.5 mL) and CHCl₃ (2.5 mL) was added under vigorous stirring. The mixture was stirred at rt for 16 h. DCM was added and the solution was washed with water and sat NaHCO₃. The organic phase was dried over MgSO₄, filtered, and volatiles were evaporated in vacuo. The crude product was purified by flash column chromatography (silica gel; EtOAc in heptane, gradient from 0/100 to 100/0). The desired fractions were collected and concentrated in vacuo to yield I-36 (373 mg, 32%) as a yellow solid.

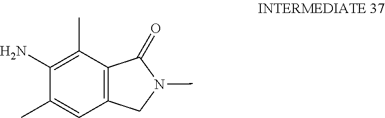

I-36 (323 mg, 1.009 mmol) and methylboronic acid (302.125 mg, 5.047 mmol) was added to a stirred solution of 1,4-dioxane (8 mL), water (2 mL), and sodium carbonate (641.93 mg, 6.06 mmol). PdCl₂(dppf) (82.638 mg, 0.101 mmol) was added. The reaction mixture was stirred overnight at 105° C. Water and EtOAc were then added. The organic layer was separated, dried (MgSO₄) and filtered and the solvents were evaporated in vacuo. The crude mixture was purified by flash column chromatography (silica; EtOAc in heptane, gradient from 0/100 to 50/50). The desired fractions were collected and concentrated in vacuo to yield I-37 (107 mg, 56%) as an orange solid.

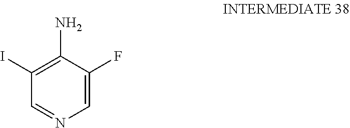

4-Amino-3-fluoropyridine [2247-88-3] (3 g, 26.76 mmol) and N-iodosuccinimide [516-12-1] (6.081 g, 27.028 mmol) was dissolved in DMF (51.802 mL, 669.01 mmol) and stirred at rt for 12 h then at 70° C. for 3 days. Then, additional N-iodosuccinimide (3.0 g, 13.4 mmol) was added each day for 2 days and the reaction was stopped after 50% conversion. The solvent was concentrated in vacuo. The crude was dissolved in EtOAc and washed with a sat sol of NaHSO₃. The organic layer was dried (MgSO₄), filtered and concentrated. A second purification was performed by flash column chromatography (silica, heptane/EtOAc, gradient from 100/0 to 50/50) to yield I-38 (1.7 g, 27%) as a white solid.

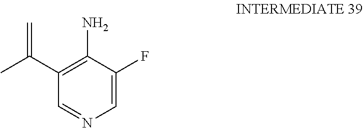

A mixture of I-38 (350 mg, 1.471 mmol), isoprenylboronic acid pinacol ester [126726-62-3] (414.632 μL, 2.21 mmol) and Pd(PPh₃)₄ (169.937 mg, 0.15 mmol) in NaHCO₃ sat. solution (2 mL) and 1,4-dioxane (3.76 mL, 44.1 mmol) was stirred and heated under nitrogen atmosphere for 15 min at 130° C. in a MW. The mixture was treated with sat. NaHCO₃ and extracted with EtOAc. The organic layer was separated, dried (MgSO₄), filtered and the solvents were evaporated in vacuo. The product was purified flash column chromatography (silica, heptane/EtOAc, gradient from 100/0 to 50/50) to obtain I-39 (205 mg, 92%) as a colourless oil.

In an analogous manner, the following intermediates were synthesized from the indicated starting materials and reagents

| STARTING MATERIAL | REAGENT | INTERMEDIATE |
|---|---|---|
| 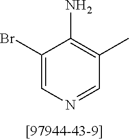 | 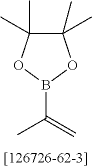 | 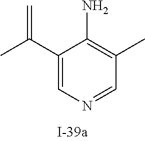 |
| 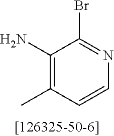 | 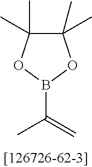 | 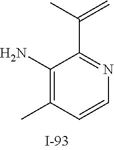 |

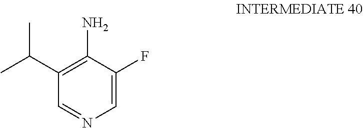

To a solution of I-39 (205 mg, 1.347 mmol) in EtOH (23.205 mL) was added Pd/C (10%, 1.434 g, 1.347 mmol). The mixture was stirred under hydrogen atmosphere for 1 h. The solvent was evaporated in vacuo to obtain I-40 (202.5 mg, yield 97%) as a colorless liquid.

In an analogous manner, the following intermediates were synthesized from the indicated starting materials and reagents

| STARTING MATERIAL | INTERMEDIATE |
|---|---|
| I-39a | 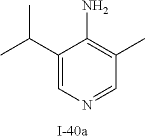 |
| I-93 | 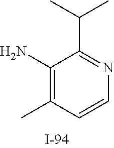 |

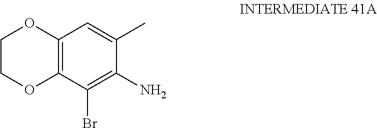

2,3-Dihydro-7-methyl-1,4-benzodioxin-6-amine [59820-84-7] (0.3 g, 1.816 mmol) was dissolved in acetic acid (10 mL). Then acetic acid (2 mL) solution containing Br₂ (0.102 mL, 1.998 mmol) was dropped into the solution under vigorous stirring. The mixture was stirred at rt for 4 h. CHCl₃ (10 mL) was added in the mixture. DCM was added and the solution was washed with water. The combined organic extracts were dried (MgSO₄), filtered and all volatiles were evaporated in vacuo. The crude product was purified by flash column chromatography (silica; EtOAc in heptane, gradient from 0/100 to 20/80). The desired fractions were collected and concentrated in vacuo to yield I-41 (333 mg, 75%) as a yellow solid.

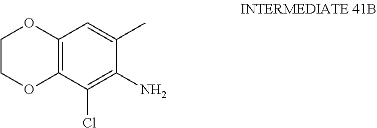

2,3-Dihydro-7-methyl-1,4-benzodioxin-6-amine [59820-84-7] (0.3 g, 1.816 mmol) was dissolved in acetic acid (10 mL). Then N-chlorosuccinimide (266.76 mg, 1.998 mmol) was added and the mixture was stirred at RT for 16 h. DCM was added and the solution was washed with water. The organic phase was washed with NaHCO₃, dried over MgSO₄, filtered, and all volatiles were evaporated in vacuo. The crude product was purified by flash column chromatography (silica; EtOAc in heptane, gradient from 0/100 to 40/60). The desired fractions were collected and concentrated in vacuo to yield I-41b (117 mg, 32%) as a yellow solid.

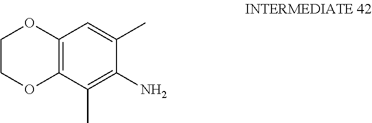

I-41a (233 mg, 0.96 mmol) and methylboronic acid (142.85 mg, 2.39 mmol) was added to a stirred solution of 1,4-dioxane (8 mL), water (2 mL), and sodium carbonate (303.52 mg, 2.86 mmol). PdCl₂(dppf) (39.07 mg, 0.048 mmol) was added. The reaction mixture was stirred overnight at 100° C. Then, methylboronic acid (142.85 mg, 2.39 mmol), sodium carbonate (303.52 mg, 2.86 mmol), and PdCl₂(dppf) (39.07 mg, 0.048 mmol) were added at rt, and the reaction mixture was stirred for 16 h at 105° C. Water and EtOAc were added, the organic layer was separated, dried (MgSO₄) and filtered and the solvents evaporated in vacuo. The crude was purified by flash column chromatography (silica; EtOAc in heptane, gradient from 0/100 to 50/50). The desired fractions were collected and concentrated in vacuo to yield I-42 (94 mg, 55%) as a solid.

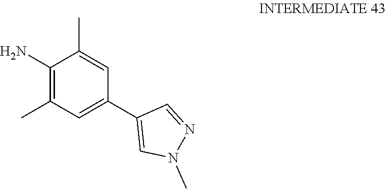

A solution of 4-bromo-2,6-dimethyl-benzenamine (400 mg, 2.0 mmol), 1-methyl-1H-pyrazole-4-boronic acid (302.098 mg, 2.40 mmol) and sodium carbonate (1M aq., 1.999 mL, 1.999 mmol) in 1,4-dioxane (10 mL) was bubbled with N2 for 5 min. Then PdCl₂(dppf) (81.63 mg, 0.1 mmol) was added and the mixture reaction was stirred for 6 h at 100° C. Water was then added and the mixture was extracted with EtOAc. The combined organic layers were dried over MgSO₄, filtered and evaporated in vacuo. The crude was purified by flash chromatography (silica; EtOAc in heptane, gradient from 0/100 to 60/40) to yield I-43 (160 mg, 40%) as a white solid.

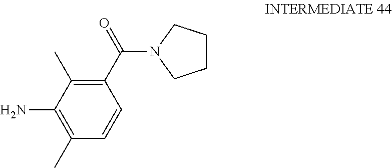 [SMILES: Cc1ccc(C(=O)N2CCCC2)c(C)c1N]

HATU [148893-10-1] (503.1 mg, 1.323 mmol) was added to a solution of 3-amino-2,4-dimethyl-benzoic acid [64289-45-8] (154 mg, 0.932 mmol), pyrrolidine [123-75-1](110 μL, 1.305 mmol) and triethylamine (260 μL, 1.865 mmol) in DCM (3 mL) while stirring at rt, and the reaction mixture was stirred for 48 h. The mixture was poured into a K₂CO₃ solution and the organic layer was separated. The aqueous phase was extracted twice with DCM. The organic layers were combined, dried over MgSO₄, filtered and concentrated. The crude intermediate was purified via Prep HPLC (stationary phase: RP XBridge Prep C18 OBD-10 μm, 30×150 mm, mobile phase: 0.25% NH₄HCO₃ solution in water, MeOH) to yield I-44 (139.6 mg, yield 68.597%) as a yellow oil.

In an analogous manner, the following intermediates were synthesized from the indicated starting materials and reagents.

| STARTING MATERIAL | REAGENT | INTERMEDIATE |
|---|---|---|
| 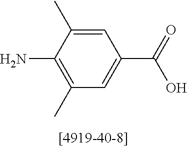 | pyrrolidine | 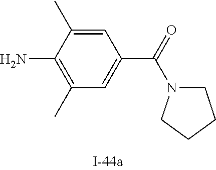 [SMILES: Cc1cc(C(=O)N2CCCC2)cc(C)c1N] |
| 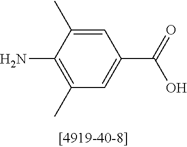 | morpholine | 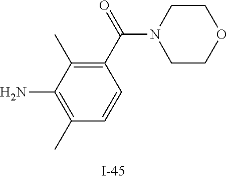 [SMILES: Cc1ccc(C(=O)N2CCOCC2)c(C)c1N] |
| 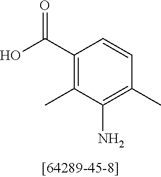 | morpholine | 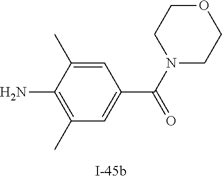 [SMILES: Cc1cc(C(=O)N2CCOCC2)cc(C)c1N] |
| 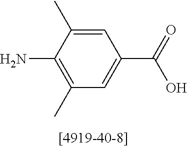 | CH₃NH₂ | 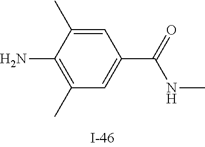 |
| 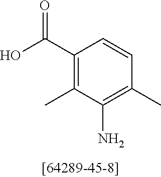 | CH₃NH₂ | 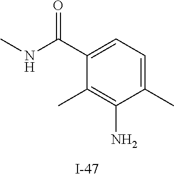 |
| 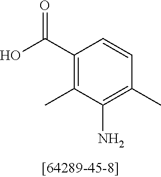 | (CH₃)₂NH | 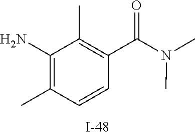 |
| 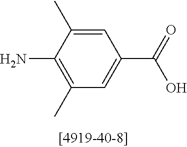 | (CH₃)₂NH | 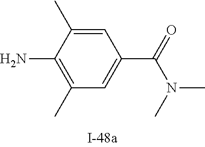 |

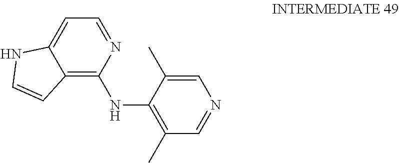 [SMILES: Cc1cncc(C)c1Nc1nccc2[nH]ccc12]

To a solution of 1-(phenylsulfonyl)-4-bromo-5-azaindole [1257294-40-8] (1 g, 2.9 mmol), in tert-butanol (12 mL) were added 3,5-dimethylpyridin-4-amine (398.5 mg, 3.3 mmol) and cesium carbonate (2.2 g, 6.5 mmol), and the resulting solution was degassed with nitrogen. To this reaction mixture were added Pd(OAc)₂ (67 mg, 0.297 mmol) and Xantphos (171.6 mg, 0.297 mmol) and the resulting solution was heated at 120° C. for 1 h. The solvent was removed in vacuo and the crude was diluted with water, extracted with DCM, dried over MgSO₄, and concentrated in vacuo. The crude mixture was purified by flash column chromatography (silica, DCM/(NH₃ in MeOH), gradient from 100/0 to 97/3) to afford I-49 (43 mg, 6%).

In an analogous manner, the following intermediate was synthesized from the indicated starting materials and reagents.

| STARTING MATERIAL | REAGENT | INTERMEDIATE |
|---|---|---|
| [1257294-40-8] | 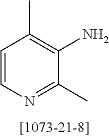 | 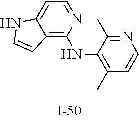 [SMILES: Cc1ccnc(C)c1Nc1nccc2[nH]ccc12] |

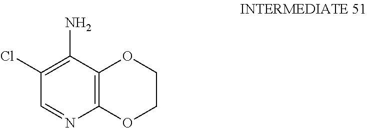 [SMILES: Nc1c(Cl)cnc2c1OCCO2]

A mixture of tert-butyl N-(7-chloro-2,3-dihydro-[1,4]dioxino[2,3-b]pyridin-8-yl)carbamate [1346447-03-7] (480 mg, 1.67 mmol) in HCl (6M in i-PrOH, 15 mL, 90 mmol) was stirred at rt for 2 h. The solvent was evaporated and the residue dissolved in water, taken up in water, and basified using K₂CO₃. The solution was extracted with DCM, dried over MgSO₄, filtered and evaporated to afford I-51 (307 mg, 98%) as a colourless oil.

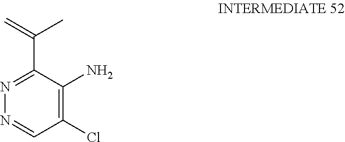

To a mixture of 3,5-dichloropyridazin-4-amine [53180-76-0] (1000 mg, 6.1 mmol) in DME (25 mL) and an aqueous solution of K₂CO₃ (12.5 mL) were added isoprene boronicacid pinacolester [126726-62-3] (1.13 g, 6.7 mmol) and Pd(PPh₃)₄ (422.79 mg, 0.37 mmol). The resulting mixture was stirred and heated under nitrogen atmosphere for 90 min at 120° C. in a pressure tube. The solvent was evaporated and the residue was taken up in water and extracted with DCM. The combined organic extracts were dried over MgSO₄, filtered and evaporated. The residue was purified by flash column chromatography (silica, heptane/EtOAc, gradient from 100/0 to 50/50). The pure fractions were evaporated to afford I-52 (930 mg, 89.9%) as a brown solid

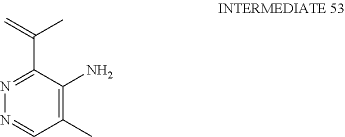

A mixture of I-52 (810 mg, 4.78 mmol), methyl zinc chloride [5158-46-3] (4.78 mL, 2 M, 9.55 mmol) and Pd(t-Bu₃P)₂ [53199-31-8] (366.09 mg, 0.72 mmol) in dry THF (20 mL) was stirred at room temp for 2 h. The reaction was quenched with the addition of NH₄Cl sat. solution and the mixture was evaporated till water. The aqeuous phase was extracted with DCM, dried over MgSO₄, filtered and evaporated. The residue was purified by flash column chromatography (silica gel, DCM/MeOH, gradient from 100/0 to 90/10). The pure fractions were evaporated, yielding I-53 (126 mg, 28.65%) as a white solid.

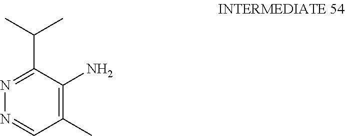

To a solution of I-53 (126 mg, 0.85 mmol) in MeOH (22 mL) was added Pd/C (10%, 90 mg, 0.085 mmol). The mixture was stirred under hydrogen atmosphere for 1 h. The solvent was evaporated in vacuo to obtain I-54 (120 mg, 94%) as a white solid.

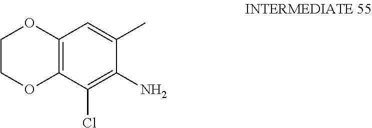

N-Chlorosuccinimide (266 mg, 1.8 mmol) was added to a solution of 2,3-dihydro-7-methyl-1,4-benzodioxin-6-amine ([59820-84-7], 300 mg, 1.8 mmol) in acetic acid (10 mL) and CHCl₃ (10 mL). The mixture was stirred at room temperature for 16 h. DCM was added and the solution was washed with water, NaHCO₃ and dried over MgSO₄. The solution was filtered, and all volatiles were evaporated in vacuo. The crude product was purified by flash column chromatography (silica; EtOAc in heptane, gradient from 0/100 to 40/60). The desired fractions were collected and concentrated in vacuo to I-55 (117 mg, 32%) as a yellow solid.

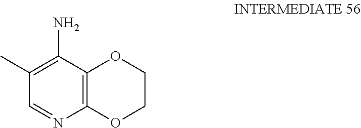

To a solution of I-51 (207 mg, 1.11 mmol) in THF (10 mL) were added methylzinc chloride [5158-46-3] (2 M, 1.11 mL, 2.22 mmol) and Pd(t-Bu₃P)₂ (85.04 mg, 0.17 mmol) and the mixture was stirred at room temp for 2 h. Additional methylzinc chloride (2 M, 1.11 mL, 2.22 mmol) was added and the mixture was stirred at rt. overnight. The reaction was quenched with sat. NH₄Cl solution and extracted with EtOAc. The combined organic layers were washed with brine, dried over MgSO₄, filtered and evaporated. The residue was purified by SFC (Stationary phase: Chiralpak Daicel IC 20×250 mm; mobile phase: CO₂, EtOH+0.4 iPrNH₂) to afford I-56 (10 mg, 5.3%) as a colourless oil.

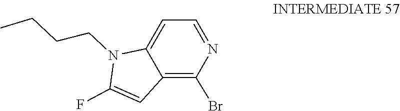

To a solution of BuLi (2.5M in hexane, 0.63 mL, 1.58 mmol) in dry THF (5.1 mL) stirred at −40° C. was added DIPA (0.28 mL, 1.98 mmol) and the mixture was stirred at −40° C. for 15 min. The RM was cooled to −78° C. and a solution of I-2 (250 mg, 0.99 mmol) in THF (10 mL) was added dropwise. The reaction mixture was stirred at −78° C. for 30 min. Then a solution of N-fluorobenzene-sulfonimide [133745-75-2] (498.29 mg, 1.58 mmol) in THF (10 mL) was added dropwise and the reaction mixture was stirred at −78° C. for 1 h and then slowly warmed to room temp over a 1 h period. The reaction mixture was decomposed with the addition of water and evaporated till water remained. The aqueous phase was extracted with DCM, dried over MgSO₄, filtered and evaporated. The residue was purified by RP chromatography, yielding I-57 (98 mg, 36.6%) as a sticky oil.

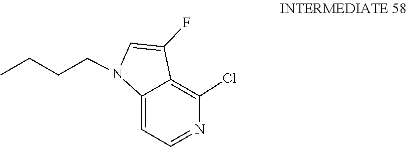

To a solution of I-1 (500 mg, 2.4 mmol) dissolved in nitroethane (10 mL) was added portion wise SelectFluor® (1697.55 mg, 4.79 mmol) at 0° C. The reaction mixture was stirred for 98 h. The mixture was quenched with ice water (20 mL) and neutralised with NaOH (1M solution in water, 1 mL). This mixture was extracted with EtOAc (twice). The combined organic layers were dried over MgSO₄, filtered and evaporated. The residue was purified by flash column chromatography (heptane/EtOAc, gradient from 90/10 to 50/50). Fractions were evaporated to afford I-58 (125 mg, 23%), as a clear oil.

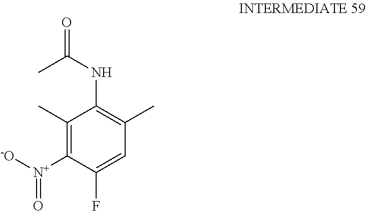

To a solution of N-(4-fluoro-2,6-dimethylphenyl)-acetamide [16643-18-8] (572 mg, 3.16 mmol) in concentrated sulfuric acid (1 mL) at −15° C. was added fuming nitric acid (136 μL, 3.18 mmol) dropwise while maintaining the temperature of the reaction at −15° C. After the addition, the reaction was stirred for 30 min and then poured into ice water. A white solid precipitate was formed which was isolated by filtration to provide the product I-59 (714 mg, 3.157 mmol).

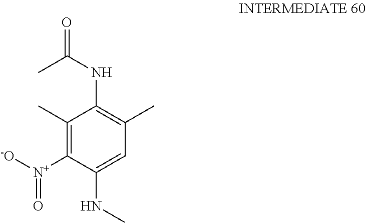

A solution of I-59 and methylamine (299 μL, 3.47 mmol) in EtOH (10 mL) was stirred for 16 h at 65° C. Then, additional methylamine (299 μL, 3.47 mmol) was added at rt and stirred for 16 h at 100° C. The solvent was evaporated. The crude product was purified by flash column chromatography (silica; EtOAc in heptane, gradient from 0/100 to 100/0). The desired fractions were collected and concentrated in vacuo to yield I-60 (621 mg, 2.6 mmol) as a yellow solid.

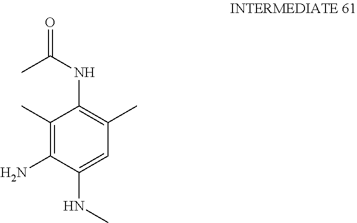

I-60 (621 mg, 2.6 mmol) was added to a stirred solution of Pd/C (10%, 69.64 mg, 0.065 mmol) in MeOH (5 mL) under nitrogen. The mixture was hydrogenated (atmospheric pressure) at room temperature for 18 h. The mixture was filtered through a pad of diatomaceous earth and the residue was washed with MeOH. The filtrate was evaporated in vacuo to yield I-61 as a white solid (534 mg, 98%).

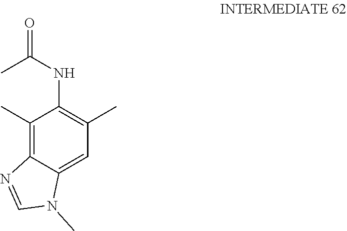

Formic acid (9 mL) was added to I-61 (534 mg, 2.6 mmol). The reaction mixture was stirred for 4 h at 100° C. The solvent was evaporated in vacuo to yield I-62 (553 mg, 98%) as a yellow solid.

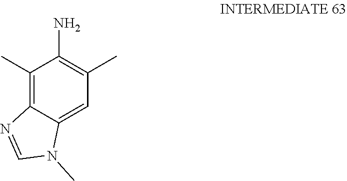

A solution of I-62 and HCl (4M in dioxane, 1.27 mL, 5.1 mmol) in MeOH (10 mL) was stirred for 16 h at 40° C. Then, additional HCl (4M in dioxane, 1.27 mL, 5.1 mmol) was added at rt, and the mixture was then stirred for an additional 16 h at 80° C. HCl (4M in dioxane, 1.27 mL, 5.1 mmol) was added daily for 10 days and the reaction mixture was stirred and heated at 80° C. The solvents were evaporated. NaHCO₃ was added and the mixture was extracted with EtOAc. The combined organic layers were dried over MgSO₄, filtered and evaporated in vacuo. The crude product was purified by flash column chromatography (silica; EtOAc in heptane, gradient from 0/100 to 100/0; then DCM/MeOH (10:1) in DCM, gradient from 0/100 to 50/50). The desired fractions were collected and concentrated in vacuo to afford I-63 (50 mg, 11%) as a brown oil.

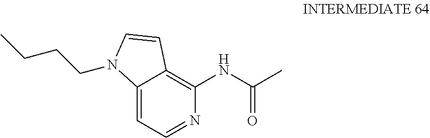 [SMILES: CCCCn1ccc2c(NC(C)=O)nccc21]

I-1 (100 mg, 0.48 mmol) and acetamide (31 mg, 0.52 mmol) were added to a stirred solution of Pd(OAc)₂ (4.3 mg, 0.019 mmol), XantPhos (24 mg, 0.043 mmol) and cesium carbonate (0.3 g, 0.96 mmol) in dioxane (8 mL) under nitrogen atmosphere. The mixture was stirred at 90° C. for 18 h. The residue was dissolved in EtOAc and water. The organic layer was washed with water, dried (MgSO₄), filtered and the solvents were evaporated in vacuo. The crude product was purified by flash column chromatography (silica, EtOAc in heptane, gradient from 0/100 to 100/0). The desired fractions were collected and concentrated in vacuo to yield I-64 (48 mg, 43%) as a sticky solid.

The following intermediate was obtained in an analogous manner to that described for I-64 from the indicated starting material.

| STARTING MATERIAL | INTERMEDIATE |
|---|---|
| 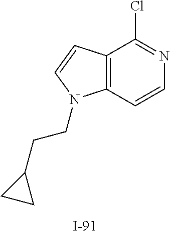 [SMILES: Clc1nccc2c1ccn2CCC1CC1] | 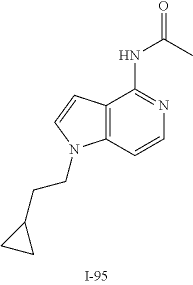 [SMILES: CC(=O)Nc1nccc2c1ccn2CCC1CC1] |

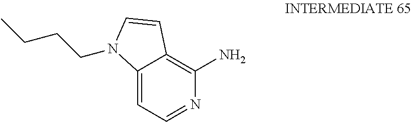

A solution of I-64 (322 mg, 1.39 mmol) and hydrochloric acid (2.27 mL, 2.78 mmol) in MeOH (2 mL) was stirred 16 h at 50° C. Then, additional hydrochloric acid (2.27 mL, 2.78 mmol) was added at rt, and the mixture was stirred for 16 h at 50° C. The solvents were evaporated. NaHCO₃ was added and the mixture was extracted with EtOAc. The combined organic layers were dried over MgSO₄, filtered and evaporated in vacuo. The crude product was purified by flash column chromatography (silica; EtOAc in heptane, gradient from 0/100 to 100/0; then DCM/MeOH (10:1) in DCM, gradient from 0/100 to 0/100). The desired fractions were collected and concentrated in vacuo to yield I-65 (100 mg, 38%) as a yellow oil.

The following intermediate was obtained in an analogous manner to that describe for I-65 from the indicated starting material

| STARTING MATERIAL | INTERMEDIATE |
|---|---|
| 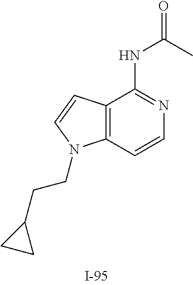 [SMILES: CC(=O)Nc1nccc2c1ccn2CCC1CC1] | 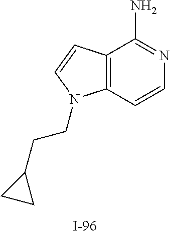 [SMILES: Nc1nccc2c1ccn2CCC1CC1] |

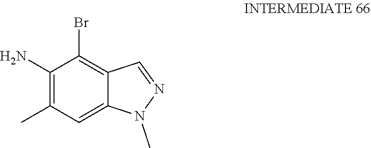

To a solution of 1,6-dimethyl-1H-indazol-5-amine ([1780910-53-3], 430 mg, 2.7 mmol) in DCM (15 mL) was added a solution of bromine (150 μL, 2.94 mmol) in DCM (5 mL). The mixture was stirred at room temperature for 16 h. DCM (30 mL) was added and the solution was washed with water. The combined organic extracts were dried over MgSO₄, filtered, and all volatiles were evaporated in vacuo. The crude product was purified by column chromatography (silica; EtOAc in heptane, gradient from 0/100 to 50/50). The desired fractions were collected and concentrated in vacuo to yield I-66 (610 mg, 95%) as a white solid.

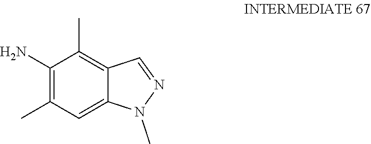

I-66 (610 mg, 2.54 mmol) and methylboronic acid (380 mg, 6.35 mmol) were added to a stirred mixture of sodium carbonate (807 mg, 7.6 mmol) in water (2 mL), and dioxane (8 mL) under nitrogen atmosphere. PdCl₂(dppf) (103 mg, 0.12 mmol) was added. The reaction mixture was stirred overnight at 105° C. Water and EtOAc were added. The organic layer was separated, dried (MgSO₄), filtered and the solvents were evaporated in vacuo. The crude was purified by flash column chromatography (silica; EtOAc in Heptane, gradient from 0/100 to 50/50). The desired fractions were collected and concentrated in vacuo to yield I-67 (330 mg, 74%) as a yellow solid.

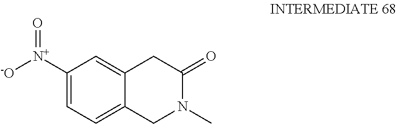

A solution of phosphorous pentoxide (1.79 g, 12.6 mmol) in methanesulfonic acid (14.9 mL, 229 mmol) was stirred for 5 h, after which N-methyl-3-nitro-benzeneacetamide [19281-10-8] (1.79 g, 12.6 mmol) and paraformaldehyde (387.7 mg, 12.6 mmol) were added under nitrogen atmosphere and the reaction mixture was stirred at 80° C. for 48 h. The reaction mixture was cooled to 0° C. and water was added. The residue was dissolved in EtOAc and the pH of the mixture was adjusted to 8 using NaOH (5M) and extracted with EtOAc. The organic phase was separated, dried (MgSO₄), filtered and the solvents were evaporated in vacuo. The crude was purified by flash column chromatography (silica, EtOAc in heptane, gradient from 0/100 to 100/0). The desired fractions were collected and concentrated in vacuo to yield I-68 (495 mg, 24%) as a white solid.

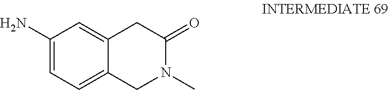

Pd/C 10% (74.8 mg, 0.07 mmol) was added to a stirred solution of I-68 (580 mg, 2.8 mmol) in MeOH (10 mL) under nitrogen atmosphere. The mixture was hydrogenated (atmospheric pressure) at room temperature for 18 h. The mixture was filtered through a pad of diatomaceous earth and the residue was washed with MeOH. The filtrate was evaporated in vacuo to yield I-69 (452 mg, 53%) as a brown solid.

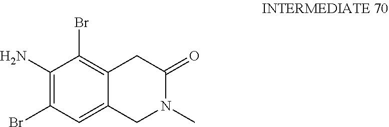

I-69 (456 mg, 2.6 mmol) was dissolved in CHCl₃ (7.5 mL) and acetic acid (7.5 mL). Then a CHCl₃ (2.5 mL) and acetic acid (2.5 mL) solution containing bromine (292 μL, 5.6 mmol) was dropped into the mixture under vigorous string. The mixture was stirred at room temperature for 5 h. DCM was added and the solution was washed with water and sat NaHCO₃, dried over MgSO₄, filtered and all volatiles were evaporated in vacuo. The crude product was purified by flash column chromatography (silica; EtOAc in heptane, gradient from 0/100 to 50/50). The desired fractions were collected and concentrated in vacuo to yield I-70 (452 mg, 52%) as a yellow solid.

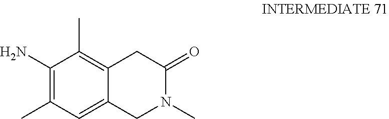

I-70 (452 mg, 1.35 mmol) and methylboronic acid (405 mg, 6.7 mmol) was added to a stirred solution of dioxane (8 mL), water (2 mL) and sodium carbonate (860 mg). PdCl₂(dppf) (110 mg, 0.135 mmol) was added and the reaction mixture was stirred overnight at 105° C. Water and EtOAc were added. The organic layer was separated, dried (MgSO₄) and filtered and the solvents were evaporated in vacuo. The crude was purified by flash column chromatography (silica; EtOAc in Heptane, gradient from 0/100 to 100/0). The desired fractions were collected and concentrated in vacuo to yield I-71 (151 mg, 54%) as a yellow solid.

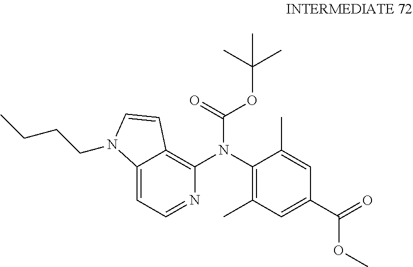 [SMILES: CCCCn1ccc2c(N(C(=O)OC(C)(C)C)c3c(C)cc(C(=O)OC)cc3C)nccc21]

To a solution of Co. No. 64 (200 mg, 0.569 mmol) in THF (19 mL) and DMF (18 mL) was added NaH (60% dispersion in mineral oil, 25 mg, 0.626 mmol) at rt. Then the reaction mixture was stirred until gas evolution stopped. Di-tert-butyl dicarbonate (136 mg, 0.626 mmol) was added portion wise and the reaction mixture was stirred at rt for 4 h and at 80° C. during 1 h. The mixture was then diluted with water and extracted with DCM. The organic layer was separated, dried (MgSO₄), filtered and the solvents were evaporated in vacuo. The crude mixture was purified by flash column chromatography (silica gel; DCM/MeOH, gradient from 100/0 to 100/0) to obtain I-72 (182 mg, 71%)

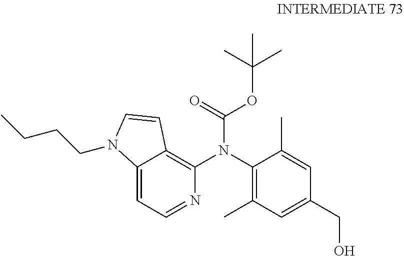 [SMILES: CCCCn1ccc2c(N(C(=O)OC(C)(C)C)c3c(C)cc(CO)cc3C)nccc21]

Lithium borohydride (2M in THF, 236 μL, 0.473 mmol) was added to a stirred solution of I-72 (178 mg, 0.394 mmol) in THF (5 mL) at 0° C. The reaction mixture was stirred at rt for 12 h. Additional lithium borohydride was added (98.5 μL) and the reaction mixture was stirred at rt for 4 h. Then Na₂SO₄.10 H₂O was added and the mixture was stirred during 1 h at rt. The solution was filtered through diatomaceous earth and washed with EtOAc. The solvents were evaporated in vacuo to afford I-73 which was used in the next step without further purification.

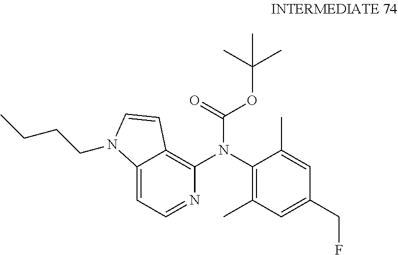 [SMILES: CCCCn1ccc2c(N(C(=O)OC(C)(C)C)c3c(C)cc(CF)cc3C)nccc21]

I-73 (170 mg, 0.401 mmol) was suspended in DCM (17 mL) and cooled down to 0° C. DAST (59 μL, 0.482 mmol) was added dropwise and the reaction mixture was stirred first at 0° C. and then at rt for 15 h. Additional DAST (14.7 μL) was added and the reaction mixture was stirred for 12 h. The reaction mixture was treated with water and extracted with DCM. The combined organic extracts were washed with water, dried (MgSO₄), filtered and concentrated in vacuo. The crude mixture was purified by Prep HPLC (Stationary phase: XBridge Prep C18 3.5 μm, 4.6×100 mm; mobile phase: 0.2% NH₄HCO₃ solution in water, MeOH) to afford I-74 (74 mg, 43%).

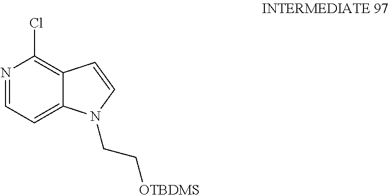 [SMILES: CC(C)(C)[Si](C)(C)OCCn1ccc2c(Cl)nccc21]

4-Chloro-1H-pyrrolo[3,2-c]pyridine [60290-21-3] (1.00 g, 6.55 mmol) was dissolved in DMF (52 mL). NaH (60% dispersion in mineral oil, 288 mg, 7.21 mmol) was added at 0° C. and the reaction mixture was stirred at room temperature. When gas evolution stopped, (2-bromoethoxy)-tert-butyldimethylsilane (2.1 mL, 9.83 mmol) was added at 0° C. The reaction mixture was stirred at room temperature for 3 h and quenched with water. The mixture was diluted with EtOAc. The aqueous layer was extracted with EtOAc (3 times). The combined organic layers were washed with brine, dried (MgSO₄), filtered and concentrated in vacuo. The residue was purified by flash column chromatography (silica, DCM/MeOH, gradient from 100:0 to 98:2) to afford I-97 (1.6 g, 79%).

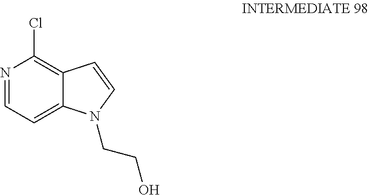 [SMILES: OCCn1ccc2c(Cl)nccc21]

I-97 (1.60 g, 5.15 mmol) was dissolved in THF (41 mL) and TBAF (1M in THF, 6.7 mL, 6.70 mmol) was added. The reaction mixture was stirred at room temperature for 1 h and concentrated in vacuo. The residue was taken up with NaHCO₃ (sat., aq.) and extracted with DCM. The organic layer was dried (MgSO₄), filtered and evaporated in vacuo. The crude mixture was purified by flash column chromatography (silica, DCM/MeOH, gradient from 100:0 to 95:5) to afford I-98 (950 mg, 94%).

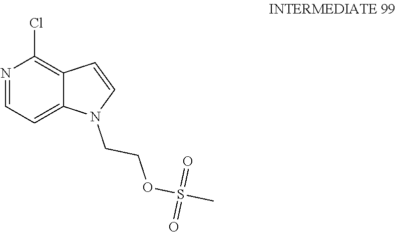 [SMILES: CS(=O)(=O)OCCn1ccc2c(Cl)nccc21]

A stirred solution of I-98 (300 mg, 1.53 mmol) in DCM (20 mL) and DMF (5 mL) was cooled to 0° C. Et₃N (0.28 mL, 1.98 mmol) was added followed by MsCl (0.13 mL, 1.68 mmol). The reaction mixture was stirred at this temperature for 1 h and quenched with water. The aqueous phase was extracted with DCM. The organic layers were dried (MgSO₄), filtered and concentrated in vacuo to afford I-99 which was used as such in the next step.

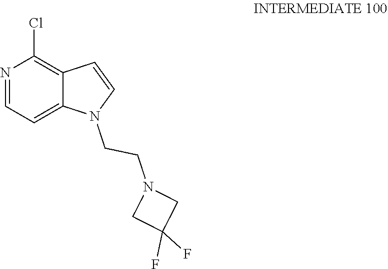 [SMILES: FC1(F)CN(CCn2ccc3c(Cl)nccc32)C1]

A mixture of I-99 (419 mg, 1.53 mmol), 3,3-difluoroazetidine hydrochloride [288315-03-7] (296 mg, 2.29 mmol), Et₃N (2.1 mL, 15.3 mmol) and KI (253 mg, 1.53 mmol) in DMF (10 mL) was stirred at 60° C. The reaction mixture was cooled to room temperature and diluted with EtOAc. The mixture was washed with water and brine. The organic fraction was dried (MgSO₄), filtered and concentrated in vacuo. The crude mixture was purified by flash column chromatography (silica, DCM/MeOH, gradient from 100:0 to 98:2) to afford I-100 (90 mg, 22%).

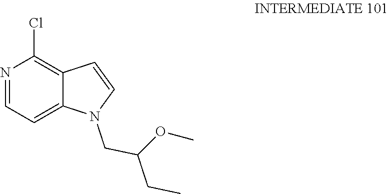 [SMILES: CCC(Cn1ccc2c(Cl)nccc21)OC]

I-22 (500 mg, 1.78 mmol) was dissolved in DMF (7 mL). NaH (60% dispersion in mineral oil, 78 mg, 1.96 mmol) was added at 0° C. and the mixture was stirred at room temperature. When gas evolution stopped, Mel (222 μL, 3.56 mmol) was added at 0° C. and the reaction mixture was stirred at room temperature for 6 h, quenched with water and diluted with EtOAc. The aqueous layer was extracted with EtOAc (3 times). The combined organic layers were washed with brine, dried (MgSO₄), filtered and concentrated in vacuo. The crude mixture was purified by flash column chromatography (silica, heptane/EtOAc, gradient from 100:0 to 80:20) to afford I-101 (300 mg, 28%).

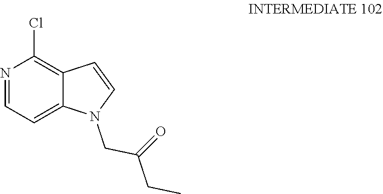

To a solution of I-22 (1.36 g, 6.05 mmol) in DCM (30 mL) was added Dess-Martin periodinane (2.70 g, 6.54 mmol) at 0° C. The reaction mixture was stirred at room temperature for 1 h. The reaction was quenched with NaHCO₃ (sat., aq.) and Na₂S203 (sat., aq.). The mixture was stirred for 30 min. The organic layer was separated, washed with brine, dried (MgSO₄), filtered and the solvent was removed in vacuo. The crude mixture was purified by flash column chromatography (silica, DCM/MeOH, gradient from 100:0 to 98:2) to afford I-102 (416 mg, 31%).

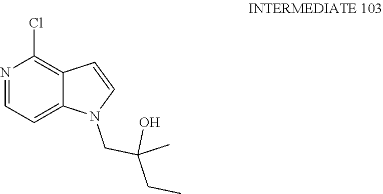 [SMILES: CCC(C)(O)Cn1ccc2c(Cl)nccc21]

MeMgBr (3M solution, 0.3 mL, 0.9 mmol) was added to a solution of I-102 (100 mg, 0.45 mmol) in THF (1 mL) at 0° C. The reaction mixture was stirred for 3 h, and NH₄Cl (sat., aq.) was added. The mixture was extracted with EtOAc. The combined organic extracts were dried (Na₂SO₄), filtered and concentrated in vacuo. The crude mixture was purified by flash column chromatography (silica, DCM/MeOH, gradient from 100:0 to 98:2) to afford I-103 (57 mg, 53%).

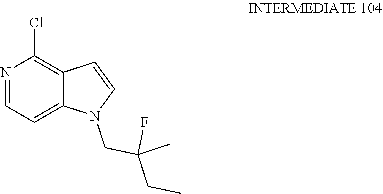 [SMILES: CCC(C)(F)Cn1ccc2c(Cl)nccc21]

I-103 (500 mg, 2.095 mmol) was suspended in DCM (40 mL) and the solution was cooled to 0° C. DAST (0.5 mL, 4.19 mmol) was added dropwise and the reaction mixture was stirred at 0° C. and then at room temperature for 3 h. The reaction was treated with water and NaHCO₃. The aqueous phase was extracted with DCM. The combined organic extracts were washed with water, dried (MgSO₄), filtered and concentrated in vacuo. The crude mixture was purified by flash column chromatography (silica, DCM) to afford I-104 (450 mg, 89%).

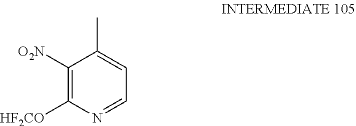

NaH (60% dispersion in mineral oil, 649 mg, 16.2 mmol) was added to a slurry of 2-hydroxy-4-methyl-3-nitropyridine [21901-18-8] (1.00 g, 6.49 mmol) in CH₃CN (70 mL) at 0° C. and under N2 atmosphere. The mixture was stirred at room temperature for 45 min, and 2,2-difluoro-2-(fluorosulfonyl)acetic acid [1717-59-5] (0.89 mL, 8.37 mmol) was added dropwise. The reaction mixture was stirred at 20° C. overnight. The reaction was quenched with NH₄Cl (sat., aq.) and extracted with EtOAc (twice). The combined organic extracts were washed with brine, dried (MgSO₄), filtered and concentrated to dryness in vacuo. The crude mixture was purified by flash column chromatography (silica, heptane/EtOAc, gradient from 100:0 to 50:50) to afford I-105 (670 mg, 51%).

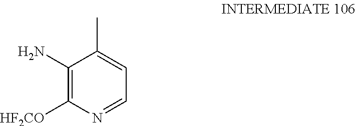

I-105 (0.81 g, 3.97 mmol) was dissolved in EtOH (22 mL), THF (7.4 mL) and water (7.4 mL). Iron (1.77 g, 31.7 mmol) and ammonium chloride (2.55 g, 47.6 mmol) were added. The reaction mixture was stirred in a sealed tube at 60° C. for 2 h. The reaction mixture was diluted EtOH and filtered through Celite®. The pad was washed with EtOH, and the filtrate was concentrated in vacuo to ˜2 mL. The solution was diluted with DCM and washed with NaHCO₃ (sat., aq.). The organic layer was dried, filtered and evaporated in vacuo to afford I-106 (685 mg, 79%, 80% purity).

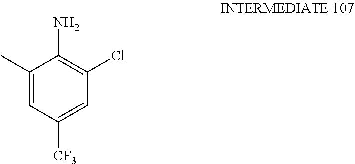

To a stirred solution of 2-methyl-4-(trifluoromethyl)aniline [67169-22-6] (5.00 g, 28.5 mmol) in DMF (50 mL) was added in small portions N-chlorosuccinimide (4.28 g, 31.4 mmol). The reaction mixture was stirred at 50° C. for 2 h, cooled and concentrated in vacuo. The residue was diluted with DCM and treated with K₂CO₃ (sat., aq.) (twice). The organic layer was dried (MgSO₄), filtered and concentrated in vacuo. The crude mixture was purified by flash column chromatography (silica, heptane/EtOAc, gradient from 100:0 to 60:40). The residue was dissolved in DIPE and treated with HCl (6M in i-PrOH) and stirred overnight. The white solid was collected by filtration and dried to afford I-107 (5.6 g, 80%).

The following intermediate was synthesized in a similar manner to that described for intermediate I-107 from the indicated starting material.

| STARTING MATERIAL | INTERMEDIATE |
|---|---|
| 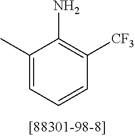 | 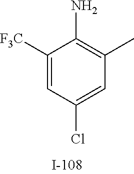 |
| 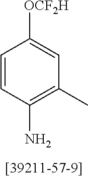 | 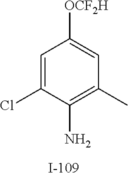 |

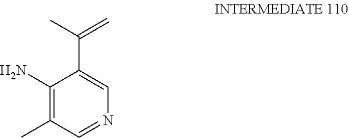

A mixture of 3-bromo-5-methylpyridine-4-amine [97944-43-9] (5.00 g, 26.7 mmol), isopropenylboronic acid pinacol ester (6.70 g, 39.9 mmol), Pd(PPh₃)₄ (3.20 g, 2.71 mmol) and NaHCO₃ (sat., aq. 50 mL) in 1,4-dioxane (50 mL) was stirred under reflux for 16 h. The suspension was cooled down and diluted with water and DCM until clear phase separation. The aqueous phase was extracted with DCM. The combined organic extracts were dried (MgSO₄), filtered and concentrated in vacuo. The crude mixture was purified by flash column chromatography (silica, DCM/(7N NH₃ in MeOH), gradient from 100:0 to 97:3).

The residue was combined with another fraction (10 mmol) and the mixture was dissolved in i-PrOH (20 mL) and treated with HCl (6M in i-PrOH, 9 mL, 54 mmol). The mixture was stirred over the weekend, ice-cooled and the product was collected by filtration to afford I-110 (4.5 g, 76%) as a white solid.

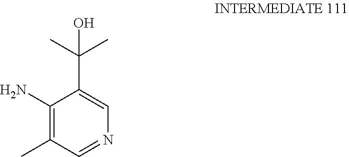

I-110 (1.50 g, 8.12 mmol) was cooled to 10° C. and H2SO₄ (50% in H₂O, 3.4 mL) was added dropwise over 10 min. The reaction mixture was stirred at 0° C. over the weekend. The mixture was added to an ice-cold solution of NaOH (100 mL). K₂CO₃ was added and the aqueous phase was extracted with CHCl₃. The mixture was concentrated in vacuo. The residue was taken up in Et₂O and stirred at room temperature. The resulting solid was filtered off and dried to afford I-111 (449 mg, 33%).

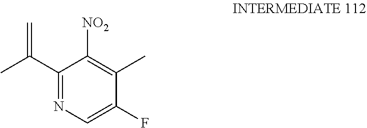

A sealed tube was charged with 3-bromo-5-methylpyridin-4-amine [97944-43-9] (1.00 g, 4.26 mmol), isopropenylboronic acid pinacol ester [126726-62-3] (1.07 g, 6.34 mmol), Pd(PPh₃)₄ (507 mg, 0.43 mmol), 1,4-dioxane (10 mL) and NaHCO₃ (sat., aq., 10 mL). The reaction mixture was stirred under reflux for 16 h, cooled down and diluted with water and DCM until clear phase separation. The aqueous phase was extracted with DCM. The combined organic extracts were dried (MgSO₄), filtered and concentrated in vacuo to afford I-112 (1.77 g, 83%, 39% purity) which was sued as such in the next step.

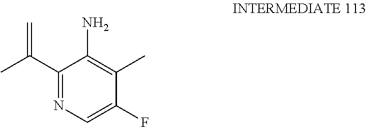

I-112 (1.77 g, 3.52 mmol) was dissolved in MeOH (20 mL), H₂O (10 mL) and THF (20 mL). Iron (4.25 g, 76.1 mmol) and NH₄Cl (5.24 g, 98.0 mmol) were added and the reaction mixture was stirred at 63° C. for 2 h. The mixture was cooled and diluted with DCM and NaHCO₃ (sat., aq.). Dicalite was added. The mixture was filtered and the filtered cake was washed with DCM. The organic layer was separated and evaporated in vacuo. The residue was treated with HCl and washed with DCM. The aqueous layer was basified with NaHCO₃ and extracted with DCM. The combined organic extracts were dried (MgSO₄), filtered and evaporated in vacuo. The crude mixture was purified by flash column chromatography (silica, heptane/EtOAc, gradient from 100:0 to 70:30) to afford I-113 (467 mg, 80%).

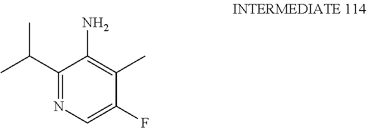

To a solution of I-113 (233 mg, 1.40 mmol) in THF (17 mL) was added platinum (5.46 mg, 0.03 mmol) and the reaction mixture was stirred at room temperature for 1 h under H2 atmosphere. The reaction mixture was filtered and the filtrate was evaporated in vacuo. The residue was combined with another fraction (1.4 mmol) and purified by flash column chromatography (silica, DCM/MeOH, gradient from 100:0 to 90:10) to afford I-114 (224 mg, 48%).

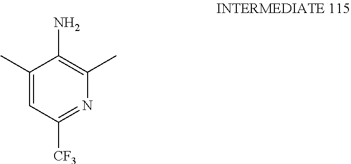

2,4-Dibromo-6-(trifluoromethyl)pyridine-3-amine [1214365-67-9] (900 mg, 2.81 mmol) was dissolved in 1,4-dioxane (7.2 mL) and water (0.9 mL). Trimethylboroxine [823-96-1] (1.13 mL, 8.07 mmol), Pd(dppf)Cl₂.DCM (206 mg, 0.25 mmol) and K₂CO₃ (1.17 g, 8.47 mmol) were added to the solution and the reaction mixture was stirred at 140° C. for 1 h in a microwave. The crude mixture was combined with another fraction (0.31 mmol) and diluted with water and EtOAc. The aqueous layer was extracted. The combined organic extracts were washed with brine, dried (MgSO₄), filtered and evaporated in vacuo. The crude mixture was purified by flash column chromatography (silica, DCM) to afford I-115 (424 mg, 71%).

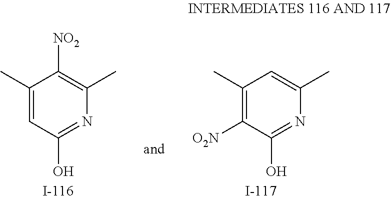

2-Amino-5-nitro-4,6-dimethylpyridine [22934-22-1] (1.43 g, 8.55 mmol) was dissolved in HCl (15% in H₂O, 22.9 mL, 274 mmol) and then cooled to 0° C. An aqueous solution of sodium nitrite (590 mg, 8.55 mmol) was added dropwise and the reaction mixture was stirred at 0° C. for 30 min, then at room temperature overnight. The mixture was extracted with CHCl₃. The organic phase was dried (MgSO₄), filtered and evaporated in vacuo to afford a mixture of I-116 and I-117 (1.15 g, 80%).

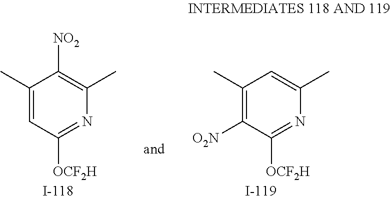

NaH (60% dispersion in mineral oil, 684 mg, 17.1 mmol) was added to a mixture of I-116 and I-117 (1.15 g, 6.84 mmol) in CH₃CN (42.2 mL) at 0° C. and under N2 atmosphere. The mixture was stirred for 45 min at room temperature and 2,2-difluoro-2-(fluorosulfonyl)acetic acid [1717-59-5] (0.94 mL, 8.83 mmol) was added dropwise. The reaction mixture was stirred at room temperature overnight and quenched with NaHCO₃ (sat., aq.). the aqueous phase was extracted with EtOAc. The combined organic extracts were dried (MgSO₄), filtered and evaporated in vacuo. the crude mixture was purified by flash column chromatography (silica, heptane/EtAOc, gradient from 100:0 to 90:10) to afford a mixture of I-118 and I-119 (1.10 g, 74%).

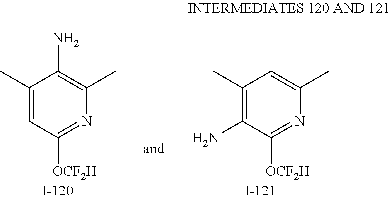

A mixture of I-118 and I-119 (1.10 g, 5.04 mmol) was dissolved in EtOH (28 mL), THF (9.4 mL) and water (9.38 mL). Iron (2.25 g, 40.3 mmol) and ammonium chloride (3.24 g, 60.5 mmol) were added. The reaction mixture was stirred at 60° C. for 2 h. The reaction mixture was diluted with EtOH and filtered through Celite®. The Celite® pad was washed with EtOH and the filtrate was concentrated in vacuo. The residue was diluted with DCM and washed with NaHCO₃ (sat., aq.). The organic layer was dried, filtered and evaporated in vacuo. The crude mixture was purified by flash column chromatography (silica, heptane/EtOAc, gradient from 100:0 to 70:30) to afford I-121 (290 mg, 31%) and I-120 (250 mg, 26%).

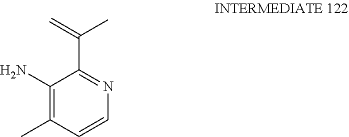

Pd(PPh₃)₄ (45.1 g, 39.03 mmol) was added to a mixture of 2-bromo-3-amino-4-methylpyridine [126325-50-6] (73.0 g, 390 mmol) and isopropenylboronic acid pinacol ester [126726-62-3] (78.7 g, 468 mmol) in 1,4-dioxane (741 mL) and NaHCO₃ (1M in H₂O, 742 mL, 742 mmol) under N2 atmosphere. The reaction mixture was stirred at 100° C. overnight. The reaction mixture was cooled to room temperature and filtered through Celite®. The filtered cake was washed with EtOAc. The layers were separated. The aqueous phase was extracted with EtOAc (twice). The combined organic extracts were washed with brine, dried (MgSO₄), filtered and concentrated in vacuo. The residue was dissolved in DCM and cooled to 0° C. HCl (2M, 400 mL, 800 mmol) was added and the resulting mixture was stirred at 0° C. for 20 min. The aqueous layer was separated and extracted with DCM (3 times). The aqueous layer was diluted with DCM (200 mL) and cooled to 0° C. Na₂CO₃ (86.9 g, 820 mmol) was added portionwise and the mixture was stirred for 5 min. Water (100 mL) was added. The mixture was stirred for another 20 min and the organic layer was separated. The aqueous layer was extracted with DCM (twice). The combined organic extracts were dried (MgSO₄), filtered and evaporated in vacuo to afford I-122 (55.7 g, 96%).

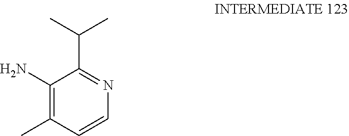

To a solution of I-122 (24.0 g, 162 mmol) in EtOH (687 mL) was added Pd/C (10%, 2.06 g, 1.94 mmol). The reaction mixture was stirred at room temperature under H2 atmosphere for 8 h. The mixture was filtered through Celite® and the filtrate was concentrated in vacuo. The crude mixture was purified by flash column chromatography (silica, DCM/MeOH gradient from 100:0 to 98:2) to afford I-123 (18.8 g, 77%).

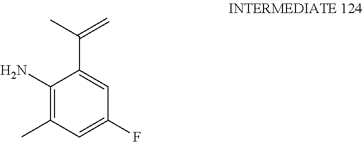

A mixture of 2-bromo-4-fluoro-6-methylaniline [202865-77-8] (2.00 g, 9.80 mmol), isopreneboronic acid pinacol ester [126726-62-3] (1.81 g, 10.8 mmol), Pd(PPh₃)₄ (680 mg, 0.59 mmol) and K₂CO₃ (sat., aq., 25 mL) in DME (40.2 mL) was stirred at 120° C. under N2 atmosphere for 90 min in a pressure tube. The mixture was concentrated in vacuo. The residue was taken up in water and DCM. The organic phase was separated, dried (MgSO₄), filtered and evaporated in vacuo. The crude mixture was purified by flash column chromatography (silica, heptane/EtOAc, gradient from 100:0 to 50:50) to afford I-124 (1.13 g, 70%) as a yellow oil.

The following intermediate was obtained in an analogous manner to that described for I-124 from the indicated starting material and reagent.

| STARTING | | |
|---|---|---|
| MATERIAL | REAGENT | INTERMEDIATE |
| 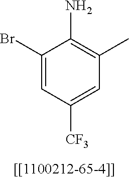 | 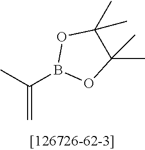 | 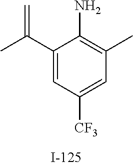 |

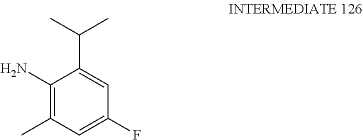

A mixture of I-124 (1.13 g, 6.84 mmol) and Pd/C (10%, 728 mg, 0.68 mmol) in MeOH (179 mL) was stirred under H2 atmosphere at room temperature for 72 h. The mixture was filtered and the filtrate was evaporated in vacuo to afford I-126 (884 mg, 77%).

The following intermediate was obtained in an analogous manner to that described for I-126 from the indicated starting material.

| STARTING MATERIAL | INTERMEDIATE |
|---|---|
| 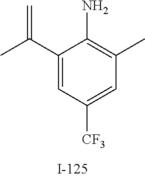 | 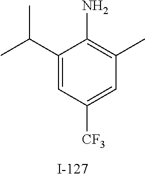 |

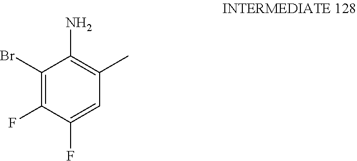

N-Bromosuccinimide [128-08-5] (3.26 g, 18.3 mmol) was dissolved in DMF (10 mL) and was added dropwise to a solution of 4,5-difluoro-2-methylaniline [875664-57-6](2.50 g, 17.5 mmol) in anhydrous DMF (21.4 mL) at 0° C. The reaction mixture was warmed to room temperature over 15 min and poured out in water. The mixture was extracted with Et₂O. The organic layer was dried (MgSO₄), filtered and evaporated in vacuo. The crude mixture was purified by flash column chromatography (silica, heptane/EtOAc, gradient from 100:0 to 70:30) to afford I-128 (1.8 g, 46%).

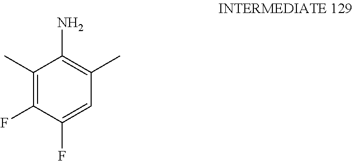

The reaction was carried out under anhydrous conditions and using dried glassware. A mixture of I-128 (650 mg, 2.93 mmol) in anhydrous THF (14.6 mL) was purged for 10 min with N2. Pd(t-Bu₃)₂P (43.9 mg, 85.9 mmol) was added and methylzinc chloride (2M solution, 2.20 mL, 4.40 mmol) was added with a syringe while maintaining the internal temperature around room temperature. The reaction mixture was stirred for 1 and water (10 mL) was added. The mixture was filtered through dicalite and the filtrate was evaporated in vacuo (water remained). The mixture was diluted with water (20 mL) and the aqueous phase was extracted with DCM. The combined organic extracts were dried (MgSO₄), filtered and concentrated in vacuo. The crude mixture was purified by flash column chromatography (silica, heptane/EtOAc, gradient from 100:0 to 70:30) to afford I-129 (430 mg, 93%).

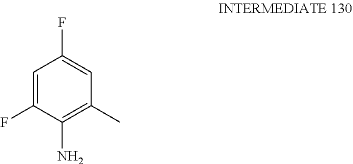

To a stirred solution of 1,5-difluoro-3-methyl-2-notrobenzene [1616526-80-7] in EtOH (200 mL) and THF (75 mL) was added solution of ammonium chloride (26 g, 0.49 mol) in H₂O (75 mL). Then iron (18 g, 0.32 mol) was added and the black suspension was vigorously stirred at 60° C. for 2 h. The mixture was cooled down and filtered over dicalite. The plug of dicalite was washed with EtOH. The filtrate was diluted with THF and filtered over a small plug of dicalite. The filtrate was diluted with brine and Et₂O.

The layers were separated. The aqueous phase was extracted with Et₂O (3 times). The combined organic extracts were washed with brine, dried (MgSO₄), filtered and concentrated in vacuo. The residue was dissolved in EtOH, treated with HCl (6N in i-PrOH) and concentrated in vacuo. The residue was suspended in DIPE to afford I-130 as a white solid (2.31 g, 32%).

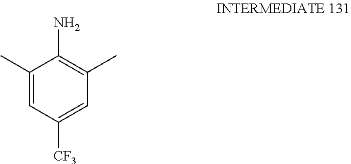

A mixture of 2,6-dibromo-4-(trifluoromethyl)aniline [72678-19-4] (5.13 g, 16.1 mmol), triemthylboroxine [823-96-1] (5 mL, 35.3 mmol), Pd(PPh₃)₄ (1.11 g, 1.00 mmol) and K₂CO₃ (sat., aq., 74 mL) in DME (74 mL) was stirred at 150° C. for 2 h. The mixture was concentrated in vacuo and the residue was taken up in water and DCM. The organic phase was separated, dried (MgSO₄), filtered and evaporated in vacuo. The crude mixture was purified by flash column chromatography (silica, heptane/EtOAc, gradient from 100:0 to 50:50) to afford I-131 (1.86 mg, 61%) as a brown oil.

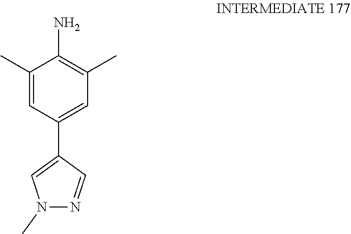

A mixture of 4-bromo-2,6-dimethylphenylamine [24596-19-8] (1.00 g, 5.00 mmol), 1-methyl-1H-pyrazole-4-boronic acid [847818-55-7] (974 mg, 5.99 mmol) and sodium carbonate (1.32 g, 12.5 mmol) in 1,4-dioxane (17 mL) was purged with N2 for 5 min. PdCl₂(dppf) (204 mg, 0.25 mmol) was added and the reaction mixture was stirred for 6 h at 90° C. The mixture was diluted with water and extracted with EtOAc. The combined organic layers were dried (MgSO₄), filtered and evaporated in vacuo. The crude mixture was purified by flash column chromatography (silica; heptane/EtOAc, gradient from 100/0 to 50/50) to afford I-177 (206 mg, 20%).

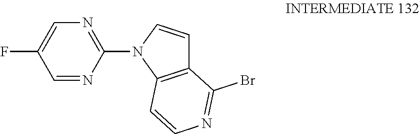 [SMILES: Fc1cnc(-n2ccc3c(Br)nccc32)nc1]

A mixture of 2-chloro-5-fluoropyrimidine [62802-42-0] (370 mg, 2.79 mmol), 4-bromo-TH-pyrrolo[2,3-d]pyridine [1000342-68-6] (500 mg, 2.54 mmol) and NaH (60% dispersion in mineral oil, 152 mg, 3.81 mmol) in DMF (30 mL) was stirred at 80° C. overnight. The reaction was quenched with water (30 mL) and extracted with DCM (3×50 mL). The combined organic layers were dried (Na₂SO₄), filtered and evaporated in vacuo. The crude mixture was purified by flash column chromatography (silica, petroleum ether/EtOAc, gradient from 100:0 to 30:10) to afford I-132 (230 mg, 31%).

The following intermediates were prepared in an analogous manner to that described for I-132 from the indicated starting materials and reagents.

| STARTING MATERIAL | REAGENT | INTERMEDIATE |
|---|---|---|
| 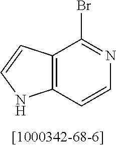 | 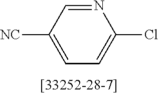 | 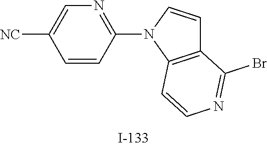 [SMILES: N#Cc1ccc(-n2ccc3c(Br)nccc32)nc1] |
| 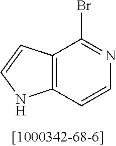 | 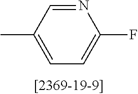 | 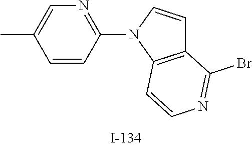 [SMILES: Cc1ccc(-n2ccc3c(Br)nccc32)nc1] |

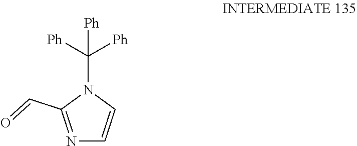 [SMILES: O=Cc1nccn1C(c1ccccc1)(c1ccccc1)c1ccccc1]

n-BuLi (2.5M solution, 5.16 mmol, 12.90 mmol) was added at 0° C. to a solution of N-tritylimidazole [15469-97-3] (2.00 g, 6.44 mmol) in THF (32 mL). The reaction mixture was stirred at 0° C. for 1.5 h and DMF (1.25 mL, 16.1 mmol) was added dropwise. The reaction mixture was stirred at 0° C. for 1 h and diluted with NH₄Cl (sat., aq.). The aqueous phase was extracted with EtOAc (twice). The combined organic layers were dried (MgSO₄), filtered and the solvents were evaporated in vacuo. The crude mixture was purified by flash column chromatography (silica, heptane/EtOAc, gradient from 100:0 to 60:40) to afford I-135 (1.52 g, 69%).

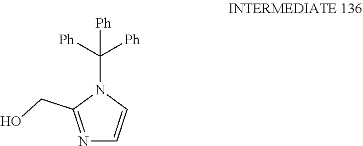 [SMILES: OCc1nccn1C(c1ccccc1)(c1ccccc1)c1ccccc1]

NaBH₄ (510 mg, 13.5 mmol) was added to a solution of I-135 (1.52 g, 4.49 mmol) in MeOH (30 mL). The reaction mixture was stirred at room temperature for 16 h. The white precipitate was filtered off and washed with CHCl₃ to afford I-136 (1.49 g, 98%). as white solid.

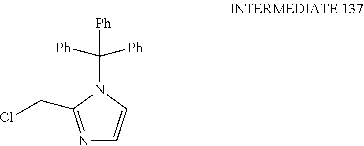 [SMILES: ClCc1nccn1C(c1ccccc1)(c1ccccc1)c1ccccc1]

Thionyl chloride (0.48 ml, 6.60 mmol) was added dropwise to a mixture of I-136 (1.50 g, 4.40 mmol) and Et₃N (1.23 mL, 8.80 mmol) in toluene (41 mL). The reaction mixture was stirred at room temperature for 1 h. Ice was added to the mixture and the aqueous phase was extracted with EtOAc (twice). The combined organic phases were washed with brine, dried (MgSO₄), filtered and concentrated in vacuo. The crude mixture was purified by flash column chromatography (silica, DCM/MeOH, gradient from 100:0 to 90:10) to afford I-137 (950 mg, 60%) as a light orange solid.

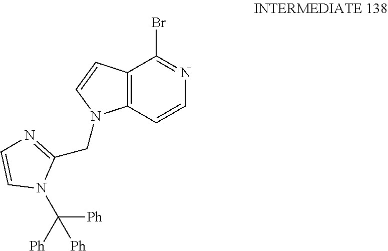 [SMILES: Brc1nccc2c1ccn2Cc1nccn1C(c1ccccc1)(c1ccccc1)c1ccccc1]

NaH (60% dispersion in mineral oil, 88.2 mg, 2.21 mmol) was added to a solution of 4-bromo-1H-pyrrolo[3,2-c]pyridine [1000342-68-6] (435 mg, 2.21 mmol) in anhydrous DMF (15 mL) under N2 atmosphere at 0° C. The mixture was stirred for 2 h and I-137 (950 mg, 2.65 mmol) was added at 0° C. The reaction mixture was warmed to room temperature and stirred for 20 h. The mixture was diluted with water and extracted with EtAOc. The organic layer was dried (MgSO₄), filtered and the solvents were evaporated in vacuo. The crude mixture was purified by flash column chromatography (silica, heptane/EtOAc, gradient from 100:0 to 0:100) to afford I-138 (854 mg, 54%, 73% purity).

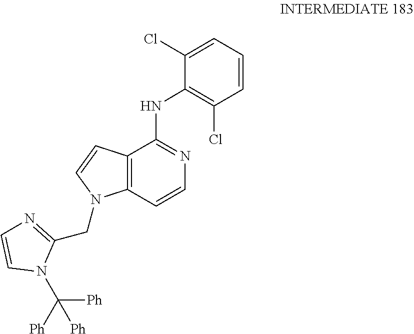 [SMILES: Clc1cccc(Cl)c1Nc1nccc2c1ccn2Cc1nccn1C(c1ccccc1)(c1ccccc1)c1ccccc1]

Pd₂dba₃ (28.2 mg, 30.8 μmol), Xantphos (44.6 mg, 0.08 mmol) and Cs₂CO₃ (376 mg, 1.16 mmol) were added to a solution of I-138 (400 mg, 0.77 mmol) in DMF (10 mL) in a sealed tube while N2 was bubbling. After 10 min, 2,6-dichloroaniline [608-31-1] (162 mg, 1.00 mmol) was added and the reaction mixture was stirred at room temperature for 10 min, and at 100° C. for 20 h. The mixture was filtrated over Celite® and the filtrate was concentrated in vacuo. The crude product was purified by flash column chromatography (silica; EtOAc in heptane, gradient from 0/100 to 100/0)). The desired fractions were collected and concentrated in vacuo to afford I-183 (152 mg, 33%).

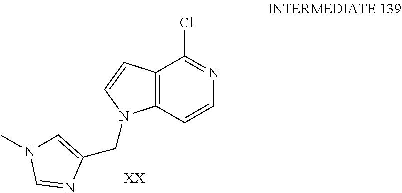

CuI (110 mg, 0.12 mmol), trans-N,N′-dimethylcyclohexane-1,2-diamine (37.9 μL, 0.24 mmol) and K₂CO₃ (332 mg, 2.40 mmol) were added to a stirred solution of 4-chloro-1H-pyrrolo[3,2-c]pyridine [60290-21-3] (238 mg, 1.56 mmol) and 4-iodo-1-methyl-1H-imidazole [71759-87-0] (250 mg, 1.20 mmol) in toluene (5 mL). The reaction mixture was stirred at 105° C. for 24 h, cooled to room temperature and partitioned between NaHCO₃ (sat., aq.) and EtOAc. The aqueous phase was extracted with EtOAc (twice). The combined organic phases were washed with brine, dried (MgSO₄), filtered and the solvents were evaporated in vacuo. The crude mixture was purified by flash column chromatography (silica, heptane/EtOAc, gradient from 100:0 to 0:100) to afford I-139 (120 mg, 43%).

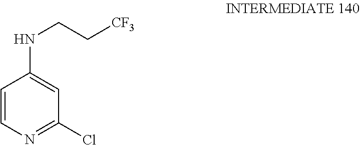 [SMILES: FC(F)(F)CCNc1ccnc(Cl)c1]

Pd₂dba₃ (356 mg, 0.39 mmol), Xantphos (375 mg, 0.65 mmol) and K₃PO₄ (4.40 g, 20.7 mmol) were added to a solution of 2-chloro-4-iodopyridine [153034-86-7] (1.55 g, 6.47 mmol) in anhydrous DMF (25 mL) in a sealed tube while N2 was bubbling. After 10 min, 3,3,3-trifluoropropylamine hydrochloride [2968-33-4] (997 mg, 6.67 mmol) was added and the reaction mixture was stirred at room temperature for 10 min, and at 70° C. for 20 h. The mixture was filtered over Celite® and the filtrate was concentrated in vacuo. The crude mixture was purified by flash column chromatography (silica, heptane/EtOAc, gradient from 100:0 to 90:10) to afford I-140 (1.06 g, 72%).

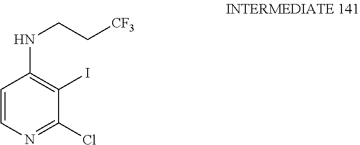 [SMILES: FC(F)(F)CCNc1ccnc(Cl)c1I]

Sodium acetate (1.16 g, 14.1 mmol) was added to a stirred solution of I-140 (1.06 g, 4.71 mmol) in acetic acid (40.7 mL). The mixture was cooled to 15° C. and iodine monochloride (236 μL, 4.71 mmol) was added dropwise. The reaction mixture was stirred at 60° C. for 24 h. The mixture was diluted with water and then the solvents were evaporated in vacuo. The residue was diluted with brine and extracted with EtOAc. The organic layer was washed with NaOH (5M) until pH 14, dried (MgSO₄), filtered and concentrated in vacuo. The crude mixture was purified by flash column chromatography (silica, heptane/EtOAc, gradient from 100:0 to 85:15) to afford I-141 (519 mg, 31%).

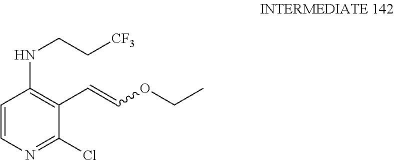 [SMILES: CCOC=Cc1c(NCCC(F)(F)F)ccnc1Cl]

PdCl₂(dppf).DCM (72.5 mg, 0.09 mmol) was added to mixture of I-141 (519 mg, 1.48 mmol), (EZ)-2-(2-ethoxyvinyl)-4,4,5,5-tetramethyl-1,3,2-dioxaborolane [1360111-87-0] (323 mg, 1.63 mmol) and LiOH.H₂O (186 mg, 4.44 mmol) in DMF (5.8 mL) at room temperature while N2 was bubbling. The reaction mixture was stirred at room temperature for 15 min and at 70° C. for 15 h. The mixture was diluted with water and extracted with EtOAc. The organic layer was washed with water, dried (MgSO₄), filtered and the solvents were evaporated in vacuo. The crude mixture was purified by flash column chromatography (silica, heptane/EtOAc, gradient from 100:0 to 40:60) to afford I-142 (389 mg, 88%).

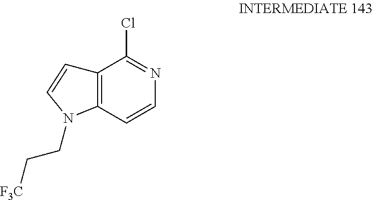 [SMILES: FC(F)(F)CCn1ccc2c(Cl)nccc21]

I-142 (389 mg, 1.25 mmol) was dissolved in acetic acid (10 mL) under N2 atmosphere. The reaction mixture was stirred at 105° C. for 5 h. The solvent was evaporated and the residue was co-distilled with toluene several times. The residue was dissolved in DCM and NaHCO₃. The organic layer was separated, dried (MgSO₄), filtered and concentrated in vacuo. The crude mixture was purified by flash column chromatography (silica, heptane/EtOAc, gradient from 100:0 to 90:10) to afford I-143 (220 mg, 70%).

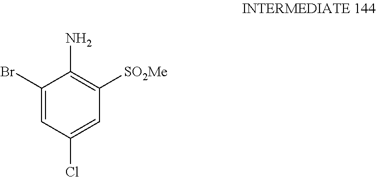 [SMILES: CS(=O)(=O)c1cc(Cl)cc(Br)c1N]

1-Amino-2-methanesulphonyl-4-chlorobenzene [102153-42-4] (660 mg, 3.21 mmol) was dissolved in DCM (20 mL). A solution of bromine (181 μL, 3.53 mmol) in DCM (20 mL) was added dropwise while vigorous stirring. The reaction mixture was stirred at room temperature for 16 h. The mixture was diluted with water. The organic layer separated and concentrated in vacuo. The crude mixture was purified by flash column chromatography (silica, heptane/EtOAc, gradient from 100:0 to 95:5) to afford I-144 (822 mg, 90%).

The following intermediate was prepared in an analogous manner to that described for I-144 from the indicated starting material.

| STARTING MATERIAL | INTERMEDIATE |
|---|---|
| 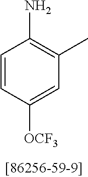 | 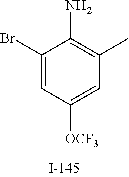 |

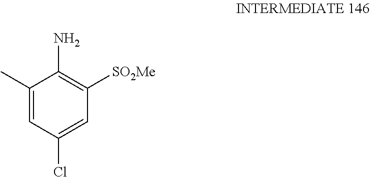

I-144 (822 mg, 2.89 mmol) was added to a stirred solution of Na₂CO₃ (918 mg, 8.66 mmol) and PdCl₂(dppf) (118 mg, 0.14 mmol) in a mixture of 1,4-dioxane (8 mL) and water (2 mL) while N2 was bubbling. The mixture was stirred at 40° C. for 5 min, then methylboronic acid (432 mg, 7.22 mmol) was added. The reaction mixture was stirred for 3 h at 105° C. The mixture was diluted with water. The aqueous phase was extracted with EtOAc. The combined organic layers were dried (MgSO₄), filtered and concentrated in vacuo. The crude mixture was purified by flash column chromatography (silica, heptane/EtOAc, gradient from 100:0 to 50:50) to afford I-146 (526 mg, 83%).

The following intermediate was prepared in an analogous manner to that described for I-146 from the indicated starting material.

| STARTING MATERIAL | INTERMEDIATE |
|---|---|
| 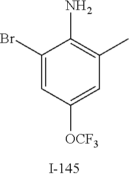 | 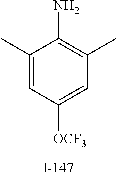 |

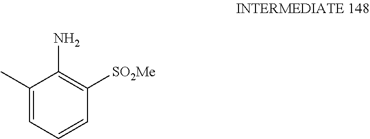

Pd/C (10%, 180 mg, 0.17 mmol) was added to a stirred mixture of I-146 (449 mg, 1.70 mmol, 83% purity) and Et₃N (0.17 mL, 1.70 mmol) in MeOH (7.60 mL). The reaction mixture was stirred under H2 atmosphere for 4 h at room temperature. The mixture was filtered through Celite® and washed with EtOAc. The filtrate was concentrated in vacuo. The crude mixture was purified by flash column chromatography (silica, heptane/EtOAc, gradient from 100:0 to 80:20) to afford I-148 (291 mg, 93%).

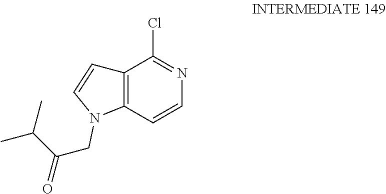 [SMILES: CC(C)C(=O)Cn1ccc2c(Cl)nccc21]

NaH (60% dispersion in mineral oil, 220 mg, 5.50 mmol) was added to a solution of 4-chloro-5-azaindole [60290-21-3] (841 mg, 5.23 mmol) in DMF (30 mL). The reaction mixture was stirred at room temperature for 30 min under N2 atmosphere. 1-Bromo-3-methyl-2-butanone [19967-55-6] (1.00 g, 5.76 mmol) was added dropwise and the reaction mixture was stirred for 16 h. The residue was dissolved with EtOAc and water. The organic layer was washed with water (twice) and brine, dried (MgSO₄), filtered and the solvents were evaporated in vacuo. The crude mixture was purified by flash column chromatography (silica, heptane/EtOAc, gradient from 100:0 to 90:10) to afford I-149 (953 mg, 76%).

The following intermediate was prepared in an analogous manner to that described for I-149 starting from the indicated starting material.

| STARTING MATERIAL | REAGENT | INTERMEDIATE |
|---|---|---|
| 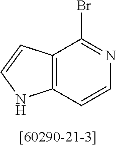 | 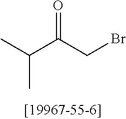 | 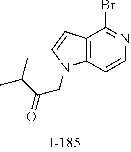 [SMILES: CC(C)C(=O)Cn1ccc2c(Br)nccc21] |

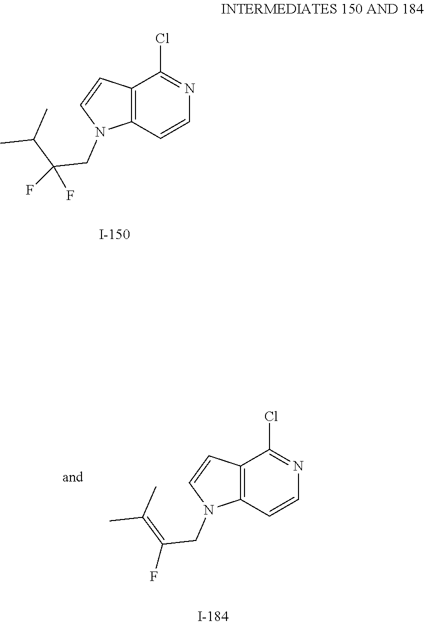 [SMILES: CC(C)C(F)(F)Cn1ccc2c(Cl)nccc21 ; CC(C)=C(F)Cn1ccc2c(Cl)nccc21]

DAST (1.97 mL, 16.1 mmol) was added to a stirred solution of I-149 (953 mg, 4.03 mmol) in anhydrous DCM (30.2 mL) under N2 atmosphere at −78° C. The reaction mixture was stirred at room temperature for 18 h. NaHCO₃ (sat., aq.) was added and the mixture was extracted with DCM (3×15 mL). The combined organic layers were dried (MgSO₄), filtered and concentrated in vacuo. The crude mixture was purified by flash column chromatography (silica, heptane/EtOAc, gradient from 100:0 to 80:20) to afford a mixture of I-150 and I-184 (686 mg, 66%).

The mixture was combined with another fraction and purified by chiral phase (Lux Cellulose-1 (150×21.2 mm, 5 um) column, mobile phase: [n-Heptane+0.1% DEA]/[2-Propanol+0.1% DEA], from 75/25 to 38/62). The desired fractions were collected and concentrated in vacuo to afford I-184 and I-150.

The following intermediate was prepared in an analogous manner to that described for I-150 and I-184 starting from the indicated starting material.

| STARTING MATERIAL | INTERMEDIATE |
|---|---|
| 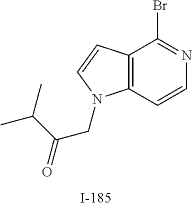 [SMILES: CC(C)C(=O)Cn1ccc2c(Br)nccc21] | 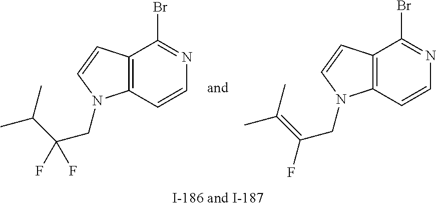 [SMILES: CC(C)C(F)(F)Cn1ccc2c(Br)nccc21 ; CC(C)=C(F)Cn1ccc2c(Br)nccc21] |

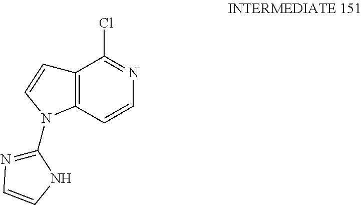 [SMILES: Clc1nccc2c1ccn2-c1ncc[nH]1]

Cs₂CO₃ (568 mg, 1.74 mmol) was added to a solution of CuI (16.2 mg, 85.1 μmol) and 1,1,1-tris(hydroxymethyl)ethane (10.2 mg, 85.1 μmol) in anhydrous 1,4-dioxane (45 mL) and anhydrous DMF (5 mL) in a sealed tube while N2 was bubbling. After 10 min, 4-chloro-1H-pyrrolo[3,2-c]pyridine [60290-21-3] (130 mg, 0.85 mmol) and 2-bromo-1H-imidazole [16681-56-4] (150 mg, 1.02 mmol) were added. The reaction mixture was stirred at room temperature for 10 min, and at 110° C. for 4 days. The mixture was filtered through Celite® and the solvents were evaporated in vacuo. The crude mixture was purified by flash column chromatography (silica, heptane/EtOAc, gradient from 100:0 to 60:40) to afford I-151 (36 mg, 18%, 35% purity).

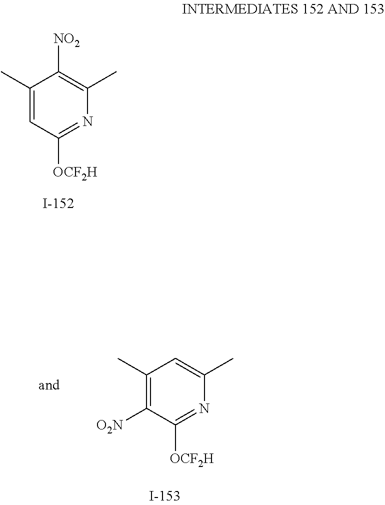

NaH (60% dispersion in mineral oil, 837 mg, 9.39 mmol) was added to a stirred solution of 5-nitro-4,6-dimethyl-pyridon-2 [22934-24-3] (1.5 g, 3.48 mmol, 39% purity) in CH₃CN. The mixture was stirred for 15 min and 2,2-difluoro-2-(fluorosulfonyl)acetic acid (0.61 mL, 5.91 mmol) was added dropwise. The reaction mixture was stirred at room temperature for 15 min and the reaction was quenched with water. CH₃CN was removed in vacuo and the residue was diluted with EtAOc. The organic layer was dried (MgSO₄), filtered and concentrated in vacuo. The crude mixture was purified by flash column chromatography (silica, heptane/EtOAc, gradient from 100:0 to 0:100) to afford a mixture of I-152 and I-153 (363 mg, 48%).

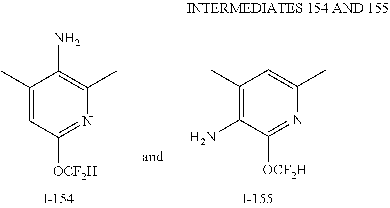

Iron (532 mg, 9.53 mmol) was added to a stirred mixture of I-152 and I-153 (260 mg, 1.19 mmol) in MeOH (13.3 mL) and H₂O (2.86 mL). The reaction mixture was stirred at 70° C. for 2 h. The mixture was cooled to room temperature and diluted with DCM. The mixture was filtered over a short pad of Celite®. The organic layer was separated, dried (MgSO₄), filtered and the solvents were evaporated in vacuo. The crude mixture was purified by flash column chromatography (silica, heptane/EtOAc, gradient from 100:0 to 85:15) to afford I-154 (85 mg, 38%) and I-155 (95 mg, 42%).

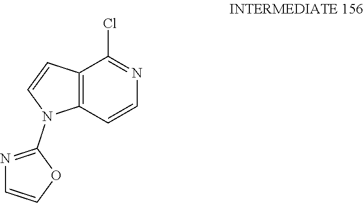 [SMILES: Clc1nccc2c1ccn2-c1ncco1]

trans-N,N′-Dimethylcyclohexane-1,2-diamine (14.5 μL, 91.8 μmol) and K₂CO₃ (127 mg, 0.92 mmol) were added to a solution of 4-chloro-1H-pyrrolo[3,2-c]pyridine [60290-21-3] (70.0 mg, 0.46 mmol) in 1,4-dioxane (6 mL) and DMF (2 mL) in a sealed tube while N2 was bubbling. The reaction mixture was stirred at room temperature for 10 min and 5-iodo-1-methyl-1H-pyrazole [34091-51-5] (125 mg, 0.64 mmol) and CuI (8.74 mg, 45.9 μmol) were added. The reaction mixture was stirred at room temperature at 110° C. for 16 h. The mixture was cooled to room temperature and partitioned between NaHCO₃ (sat., aq.) and EtOAc. The aqueous phase was extracted with EtOAc (twice). The combined organic phases were washed with brine, dried (MgSO₄), filtered and the solvents were evaporated in vacuo. The crude mixture was purified by flash column chromatography (silica, heptane/EtOAc, gradient from 100:0 to 50:50) to afford I-156 (66 mg, 65%).

The following intermediates were prepared in an analogous manner to that described for I-156 from the indicated starting materials and reagents.

| STARTING MATERIAL | REAGENT | INTERMEDIATE |
|---|---|---|
| 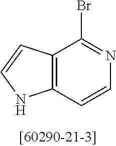 | 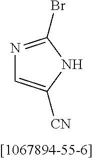 | 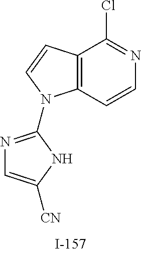 [SMILES: N#Cc1cnc(-n2ccc3c(Cl)nccc32)[nH]1] |
| 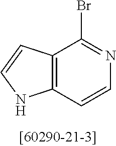 | 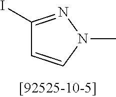 | 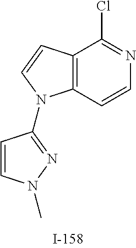 [SMILES: Cn1ccc(-n2ccc3c(Cl)nccc32)n1] |

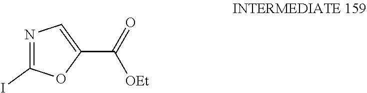 [SMILES: CCOC(=O)c1cnc(I)o1]

LiHMDS (1M solution, 15 mL, 15.0 mmol) was added at −78° C. to a solution of ethyl 5-oxazolecarboxylate [118994-89-1] (1.26 mL, 10.0 mmol) in THF (50 mL). The reaction mixture was stirred at −78° C. for 1 h and ZnCl₂ (0.7M solution, 22.8 mL, 16.0 mmol) was added dropwise. The reaction mixture was warmed to room temperature and stirred for 30 min. A solution of 12 (5.13 g, 20.0 mmol) in THF (5 mL) was added dropwise at −78° C. The reaction mixture was stirred at −78° C. for 15 min and at room temperature for 1 h. The mixture was diluted with Na₂S203 (sat., aq.) and extracted with Et₂O (twice). The combined organic extracts were dried (MgSO₄), filtered and concentrated in vacuo. The crude mixture was purified by flash column chromatography (silica, heptane/EtOAc, gradient from 100:0 to 95:5) to afford I-159 (2.2 g, 82%).

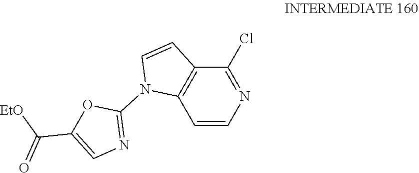 [SMILES: CCOC(=O)c1cnc(-n2ccc3c(Cl)nccc32)o1]

K₂CO₃ (362 mg, 2.62 mmol), CuI (49.9 mg, 0.26 mmol) and trans-N,N′-dimethylcyclohexane-1,2-diamine (82.7 μL, 0.52 mmol) were added to solution of 4-chloro-1H-pyrrolo[3,2-c]pyridine [60290-21-3] (200 mg, 1.31 mmol) and I-159 (420 mg, 1.57 mmol) in toluene (10 mL) in a sealed tube while N2 was bubbling. The reaction mixture was stirred at room temperature for 10 min and at 110° C. for 18 h. The mixture was cooled to room temperature and partitioned between NaHCO₃ (sat., aq.) and EtOAc. The aqueous phase was extracted with EtOAc. The combined organic phases were washed with brine, dried (MgSO₄), filtered and concentrated in vacuo. The crude mixture was purified by flash column chromatography (silica, heptane/EtOAc, gradient from 100:0 to 85:15) to afford I-160 (42 mg, 9%, 86% purity).

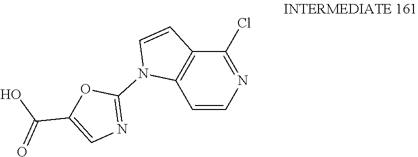 [SMILES: O=C(O)c1cnc(-n2ccc3c(Cl)nccc32)o1]

NaBH₄ (32.7 mg, 0.86 mmol) was added portionwise to a suspension of CaCl₂) (47.9 mg, 0.43 mmol) in anhydrous THF (1 mL) and EtOH (1 mL) at −20° C. under N2 atmosphere. The mixture was stirred for 15 min at −20° C. and a solution of I-160 (42.0 mg, 0.14 mmol) in anhydrous THF (1 mL) was added portionwise. The reaction mixture was stirred at −10° C. for 1 h and allowed to warm to room temperature. The reaction mixture was stirred for 16 h. The mixture was cooled to 0° C. and carefully diluted with NH₄Cl (sat., aq.) and DCM. The mixture was filtered over a pad of Celite®. The filtrate was concentrated in vacuo to afford I-161 which was used as such in the next step.

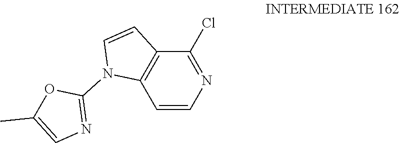 [SMILES: Cc1cnc(-n2ccc3c(Cl)nccc32)o1]

I-161 (32.0 mg, 128 μmol) was added to a stirred solution of triethylsilane (71.7 μL, 0.45 mmol) in TFA (2 mL) at room temperature. The reaction mixture was stirred at 55° C. for 18 h. The solvent was removed in vacuo. The residue was diluted with NaHCO₃ (sat., aq.) and extracted with DCM. The combined organic fractions were washed with brine, dried (MgSO₄), filtered and concentrated in vacuo. The crude mixture was purified by flash column chromatography (silica, heptane/EtOAc, gradient from 100:0 to 60:40) to afford I-162 (19 mg, 63%).

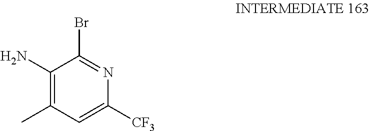

N-Bromosuccinimide (594 mg, 3.34 mmol) was added to a stirred solution of 4-methyl-6-(trifluoromethyl)pyridine-3-amine [944317-54-8] (235 mg, 1.33 mmol) in DMSO (5.6 mL) and water (310 μL). The reaction mixture was stirred at room temperature for 48 h and quenched with water. The aqueous phase was extracted with EtOAc (twice). The combined organic layers were dried (MgSO₄), filtered and concentrated in vacuo. The crude mixture was purified by flash column chromatography (silica, heptane/EtOAc, gradient from 100:0 to 85:15) to afford I-163 (209 mg, 61%).

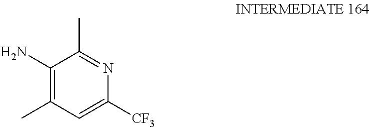

I-163 (50 mg, 0.20 mmol) and methylboronic acid (29.9 mg, 0.49 mmol) were added to a mixture of Na₂CO₃ (62.3 mg, 0.59 mmol) in 1,4-dioxane (4 mL) and H₂O (1 mL). PdCl₂(dppf) (8.00 mg, 9.8 μmol) was added and the reaction mixture was stirred at 100° C. for 16 h. The reaction mixture was diluted with water and EtOAc. The organic layer was separated, dried (MgSO₄), filtered and concentrated in vacuo. The crude mixture was combined with another fraction (0.60 mmol) and purified by flash column chromatography (silica, heptane/EtOAc, gradient from 100:0 to 90:10) to afford I-164 (122 mg, 80%).

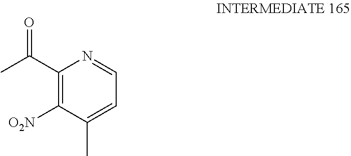

To a solution of 2-bromo-4-methyl-3-nitropyridine [23056-45-3] (6.00 g, 27.6 mmol) in toluene (264 mL) were added tributyl(1-ethoxyvinyl)tin [97674-02-7] (13.9 mL, 41.2 mmol) and Pd(PPh₃)₄ (3.20 g, 2.77 mmol). The reaction mixture was stirred at 100° C. for 16 h. HCl (37% in H₂O, 23 mL, 276 mmol) was added at 0° C. and the mixture was stirred at room temperature for 1 h. NaHCO₃ (sat., aq.) was added and the aqueous phase was extracted with Et₂O. The combined organic extracts were washed with brine, dried (Na₂SO₄), filtered and evaporated in vacuo. The crude mixture was purified by flash column chromatography (silica, heptane/EtOAc, gradient from 100:0 to 70:30) to afford I-165 (3.13 g, 63%).

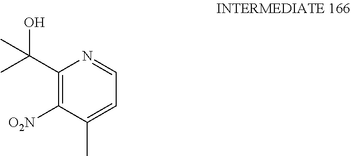

To a solution of I-165 (3.13 g, 17.4 mmol) in THF (41.5 mL) at 0° C. was added dropwise MeMgBr (1.4 M solution, 30 mL, 42 mmol). The reaction mixture was stirred at room temperature for 3 h and quenched with NH₄Cl (sat., aq.). The aqueous phase was extracted with EtOAc. The combined organic extracts were dried (Na₂SO₄), filtered and concentrated in vacuo. The crude mixture was purified by flash column chromatography (silica, DCM/MeOH, gradient from 100:0 to 99:1) to afford I-166 (736 mg, 22%).

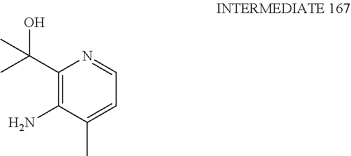

I-166 (736 mg, 3.75 mmol) was dissolved in EtOH (21 mL), THF (7 mL) and water (7 mL). iron (1.68 g, 30.0 mmol) and ammonium chloride (2.41 g, 45.0 mmol) were added and the reaction mixture was stirred in a sealed tube at 60° C. for 2 h. The reaction mixture was diluted with DCM and NaHCO₃ (sat., aq.) was added. the mixture was filtered through Celite®. The Celite® pad was washed with DCM and the filtrate was dried and evaporated in vacuo to afford I-167 (744 mg, 82%, 69% purity) which was used as such in the next step.

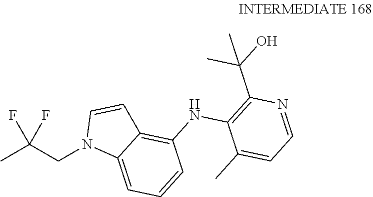 [SMILES: Cc1ccnc(C(C)(C)O)c1Nc1cccc2c1ccn2CC(C)(F)F]

A mixture of I-92 (319 mg, 1.18 mmol, 85% purity), I-167 (350 mg, 1.45 mmol, 69% purity) and Cs₂CO₃ (771 mg, 2.37 mmol) in t-BuOH (3.3 mL) was purged with N2. Pd(OAc)₂ (48.4 mg, 0.22 mmol) and Xantphos (82.3 mg, 0.14 mmol) were added and the reaction mixture was stirred at 110° C. for 1 h and at 130° C. for 2 h. The mixture was diluted with DCM and filtered over Celite®. The filtrate was concentrated in vacuo. The crude mixture was purified by flash column chromatography (silica, DCM/MeOH, gradient from 100:0 to 96:4) to afford I-168 (269 mg, 32%, 50% purity).

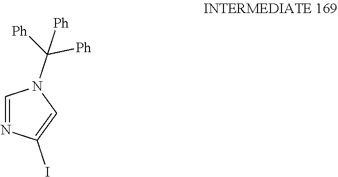 [SMILES: Ic1cn(C(c2ccccc2)(c2ccccc2)c2ccccc2)cn1]

Et₃N (0.59 mL, 4.25 mmol) was added to a solution of 4-iodoimidazole [71759-89-2](750 mg, 3.87 mmol) in DCM (30 mL). The reaction mixture was stirred at room temperature for 5 min and trytil chloride (1.19 g, 4.25 mmol) was added. The reaction mixture was stirred at 40° C. for 16 h. The reaction mixture was diluted with NaHCO₃ (sat., aq.) and extracted with DCM. The organic layer was dried (MgSO₄), filtered and the solvent were evaporated in vacuo. The crude mixture was purified by flash column chromatography (silica, heptane/EtOAc, gradient from 100:0 to 60:40) to afford I-169 (976 mg, 58%).

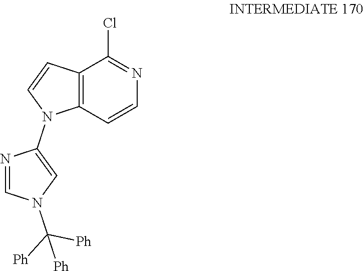 [SMILES: Clc1nccc2c1ccn2-c1cn(C(c2ccccc2)(c2ccccc2)c2ccccc2)cn1]

CuI (21.8 mg, 0.12 mmol), trans-N,N′-dimethylcyclohexane-1,2-diamine (36.1 μL, 0.23 mmol) and K₂CO₃ (317 mg, 2.29 mmol) were added to a solution of I-169 (500 mg, 1.15 mmol) in toluene (6.25 mL) in a sealed tube while N2 was bubbling. After 10 min, 4-chloro-1H-pyrrolo[3,2-c]pyridine [60290-21-3] (227 mg, 1.15 mmol) was added. The reaction mixture was stirred at room temperature for 10 min, and at 100° C. for 20 h. The reaction mixture was cooled to room temperature and diluted with NaHCO₃ (sat., aq.) and extracted with EtOAc. The organic layer was dried (MgSO₄), filtered and the solvents were evaporated in vacuo. The crude mixture was purified by flash column chromatography (silica, heptane/EtOAc, gradient from 100:0 to 80:20) to afford I-170 (430 mg, 81%).

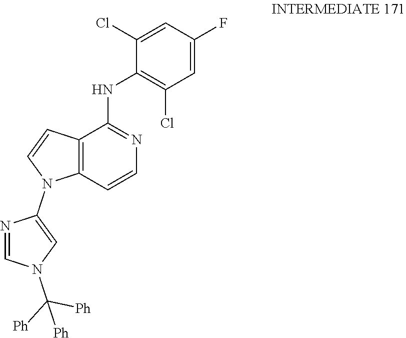 [SMILES: Fc1cc(Cl)c(Nc2nccc3c2ccn3-c2cn(C(c3ccccc3)(c3ccccc3)c3ccccc3)cn2)c(Cl)c1]

Pd₂dba₃ (34.2 mg, 37.3 μmol), Xantphos (53.9 mg, 93.3 μmol) and Cs₂CO₃ (456 mg, 1.40 mmol) were added to a mixture of I-170 (430 mg, 0.93 mmol) in DMF (12 mL) in a sealed tube while N2 was bubbling. After 10 min, 2,6-dichloro-4-fluoroaniline [344-19-4] (218 mg, 1.21 mmol) was added and the reaction mixture was stirred at room temperature for 10 min, and at 100° C. for 20 h. The mixture was filtered over Celite® and the filtrate was concentrated in vacuo. The crude mixture was purified by flash column chromatography (silica, heptane/EtOAc, gradient from 100:0 to 0:100) to afford I-171 (400 mg, 64%, 90% purity).

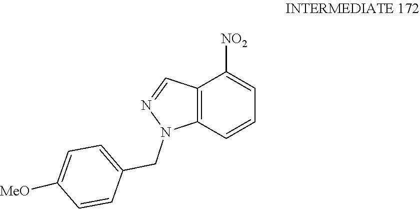 [SMILES: COc1ccc(Cn2ncc3c([N+](=O)[O-])cccc32)cc1]

Cs₂CO₃ (9.99 g, 30.7 mmol) and 4-methoxybenzyl chloride (2.5 mL, 18.4 mmol) were added to a solution of 4-nitro-1H-indazole [2942-40-7] (2.50 g, 15.3 mmol) in THF (60 mL) under N2 atmosphere. The reaction mixture was stirred at room temperature for 18 h. Additional quantity of 4-methoxybenzyl chloride (2.50 mL, 18.4 mmol) was added and the reaction mixture was stirred for another 18 h. The mixture was dissolved in water and extracted with EtOAc. The combined organic layers were dried (MgSO₄), filtered and concentrated in vacuo. The crude mixture was purified by flash column chromatography (silica, heptane/EtOAc, gradient from 100:0 to 80:20) to afford I-172 (2.15 g, 48%).

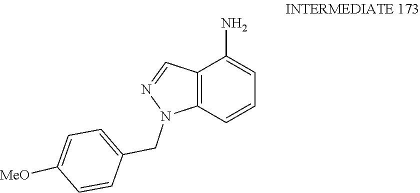 [SMILES: COc1ccc(Cn2ncc3c(N)cccc32)cc1]

Iron (3.38 g, 60.4 mmol) was added to a stirred mixture of I-172 (2.14 g, 7.55 mmol) and ammonium chloride (4.39 g, 82.1 mmol) in MeOH (84.2 mL) and H₂O (18.1 mL). The reaction mixture was stirred at 70° C. for 2 h. The mixture was cooled to room temperature and diluted with DCM. The mixture was filtered over a short pad of Celite®. The organic layer was separated, dried (MgSO₄), filtered and the solvents were evaporated in vacuo. The crude mixture was purified by flash column chromatography (silica, heptane/EtOAc, gradient from 100:0 to 65:35) to afford I-173 (1.40 g, 70%).

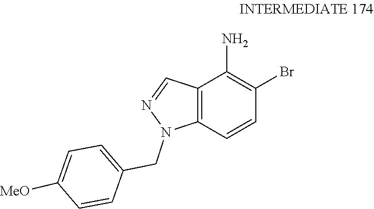 [SMILES: COc1ccc(Cn2ncc3c(N)c(Br)ccc32)cc1]

N-Bromosuccinimide (1.09 g, 6.11 mmol) was added dropwise to a solution of I-173 (1.40 g, 5.53 mmol) in CH₃CN (30 mL). The reaction mixture was stirred at 60° C. for 16 h, cooled to room temperature and diluted with NaHCO₃ (sat., aq.). The aqueous phase was extracted with EtOAc. The combined organic extracts were dried (MgSO₄), filtered and concentrated in vacuo. The crude mixture was purified by flash column chromatography (silica, heptane/EtOAc, gradient from 100:0 to 80:20) to afford I-174 (1.38 g, 74%).

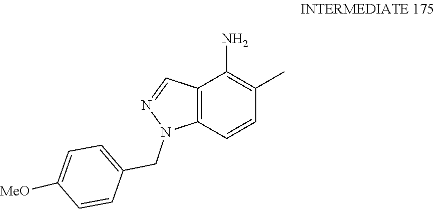 [SMILES: COc1ccc(Cn2ncc3c(N)c(C)ccc32)cc1]

I-174 (500 mg, 1.51 mmol) and methylboronic acid (450 mg, 7.53 mmol) were added to a stirred solution of Na₂CO₃ (957 mg, 9.03 mmol) in 1,4-dioxane (8 mL) and H₂O (2 mL) under N2 atmosphere. PdCl₂(dppf) (123 mg, 0.15 mmol) was added. The reaction mixture was stirred at 105° C. for 18 h in a sealed tube. The mixture was diluted with NaHCO₃ and EtOAc. The organic layer was separated, dried (MgSO₄), filtered and the solvents were evaporated in vacuo. The crude mixture was purified by flash column chromatography (silica, heptane/EtOAc, gradient from 100:0 to 80:20) to afford I-175 (288 mg, 41%, 57% purity).

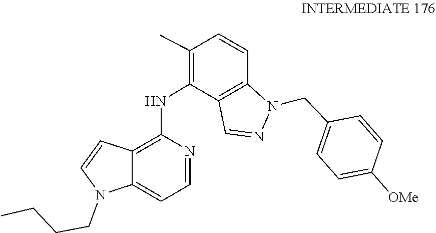 [SMILES: CCCCn1ccc2c(Nc3c(C)ccc4c3cnn4Cc3ccc(OC)cc3)nccc21]

I-175 (174 mg, 0.65 mmol) and I-2 (150 mg, 0.59 mmol) were added to a stirred mixture of Pd(OAc)₂ (5.31 mg, 23.7 μmol), Xantphos (27.4 mg, 47.3 μmol) and Cs₂CO₃ (578 mg, 1.78 mmol) in t-BuOH under N2 atmosphere. The reaction mixture was stirred at 115° C. for 8 h and diluted with EtOAC and water. The organic layer was washed with water and brine, dried (MgSO₄), filtered and the solvents were evaporated in vacuo. The crude mixture was purified by flash column chromatography (silica, heptane/EtOAc, gradient from 100:0 to 75:25) to afford I-176 (121 mg, 43%, 93% purity).

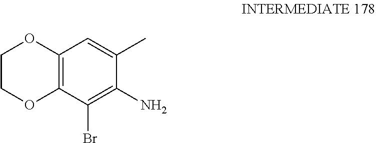

7-Methyl-2,3-dihydrobenzo[b][1,4]dioxin-6-amine [59820-84-7] (0.40 g, 2.42 mmol) was dissolved in DCM (10 mL). A solution of bromine (0.14 mL, 2.66 mmol) in DCM (2 mL) was added dropwise. The reaction mixture was stirred at room temperature for 4 h and diluted with DCM. The mixture was washed with water, dried (MgSO₄), filtered and concentrated in vacuo. The crude mixture was purified by flash column chromatography (silica, heptane/EtOAc, gradient from 100:0 to 80:20) to afford I-178 (471 mg, 80%) as a yellow solid.

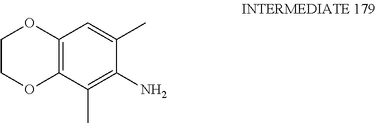

I-179 (471 mg, 1.93 mmol) and methylboronic acid (289 mg, 4.82 mmol) were added to a stirred mixture of Na₂CO₃ (613 mg, 5.79 mmol) in 1,4-dioxane (8 mL) and water (2 mL). PdCl₂(dppf) (78.9 mg, 96.5 μmol) was added. The reaction mixture was stirred at 100° C. overnight. The mixture was cooled down and additional quantity of methylboronic acid, Na₂CO₃ and PdCl₂(dppf) were added. The reaction mixture was stirred at 105° C. for another 16 h. The mixture was diluted with water and EtOAc. The organic layer was separated, dried (MgSO₄), filtered and concentrated in vacuo. The crude mixture was purified by flash column chromatography (silica, heptane/EtOAc, gradient from 100:0 to 50:50) to afford I-179 (200 mg, 58%) as a yellow solid.

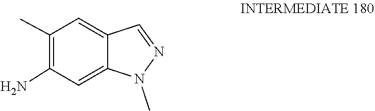

Pd/C (10% purity, 69.6 mg, 65.4 μmol) was added to a stirred solution of 1,5-dimethyl-6-nitro-1H-indazol [78416-45-2] (500 mg, 2.62 mmol) in EtOH (10 mL) under N2 atmosphere. The mixture was purged and stirred at room temperature for 18 h under H2 atmosphere. The mixture was filtered through a pad of Celite® and the residue was washed with MeOH. The filtrate was evaporated in vacuo to afford I-180 (299 mg, 71%).

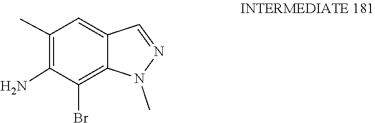

I-180 (299 mg, 1.86 mmol) was dissolved in DCM (15 mL). A solution of bromine (0.1 mL, 1.95 mmol) in DCM (4 mL) was added dropwise under vigorous stirring. The reaction mixture was stirred at room temperature for 3 h and diluted with DCM. The mixture was washed with water, dried (MgSO₄), filtered and concentrated in vacuo. The crude product was purified by flash column chromatography (silica; AcOEt in heptane, gradient from 0/100 to 20/80). The desired fractions were collected and concentrated in vacuo to afford I-181 (300 mg, 67%).

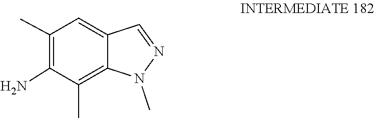

I-181 (300 mg, 1.25 mmol) and methylboronic acid (191 mg, 3.12 mmol) were added to a stirred solution of Na₂CO₃ (397 mg, 3.75 mmol) in 1,4-dioxane (4 mL) and H₂O (1 mL) under N2 atmosphere. PdCl₂(dppf) (51.0 mg, 62.5 μmol) was added and the reaction mixture was stirred at 105° C. for 16 h. The mixture was diluted with water and EtOAc. The organic layer was separated, dried (MgSO₄), filtered and the solvents were evaporated in vacuo. The crude mixture was purified by flash column chromatography (silica; EtOAc in Heptane, gradient from 0/100 to 20/80). The desired fractions were collected and concentrated in vacuo to afford I-182 (71 mg, 32%).

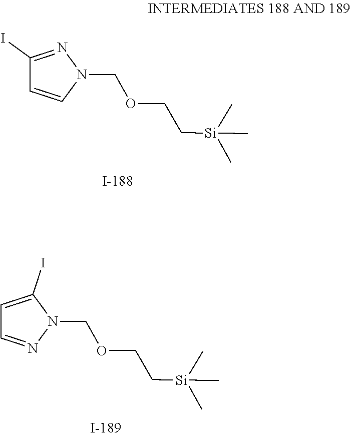

NaH (60% dispersion in mineral oil, 143 mg, 3.57 mmol) was added to a stirred solution of 3-iodo-1H-pyrazole [4522-35-4] (659 mg, 4.00 mmol) in DMF (20 mL) at 0° C. under N2 atmosphere. The mixture was stirred at room temperature for 30 min. 2-(Trimethylsilyl)ethoxymethyl chloride [76513-69-4] (0.66 mL, 3.74 mmol) was added at 0° C. and the reaction mixture was stirred at room temperature for 16 h. The mixture was diluted with water and extracted with EtOAc. The organic layer was dried (MgSO₄), filtered and the solvents were evaporated in vacuo. The crude product was purified by flash column chromatography (silica; EtOAc in heptane, gradient from 0/100 to 10/90). The desired fractions were collected and concentrated in vacuo to afford a mixture of I-188 and I-189 (965 mg, 86%).

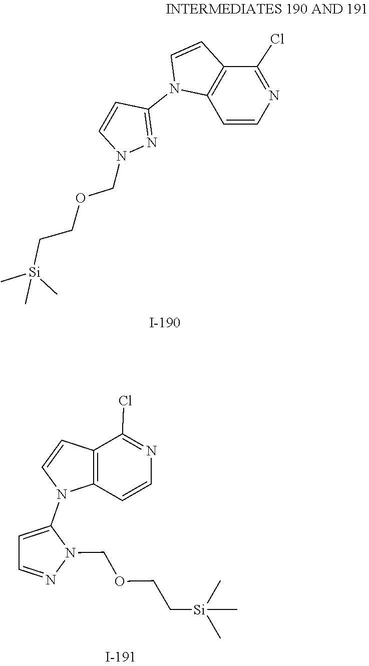 [SMILES: C[Si](C)(C)CCOCn1ccc(-n2ccc3c(Cl)nccc32)n1 ; C[Si](C)(C)CCOCn1nccc1-n1ccc2c(Cl)nccc21]

CuI (28.3 mg, 0.15 mmol), N,N′-dimethylcyclohexane-1,2-diamine (46.9 μL, 0.30 mmol) and K₂CO₃ (411 mg, 2.98 mmol) were added to a solution of I-188 and I-189 (965 mg, 2.98 mmol) in 1,4-dioxane (10 mL) in a sealed tube while nitrogen was bubbling. After 10 min, 4-chloro-1H-pyrrolo[3,2-c]pyridine [60290-21-3] (227 mg, 1.49 mmol) was added. The reaction mixture was stirred at room temperature for 10 min, and at 100° C. for 20 h. The mixture was diluted with water and extracted with EtOAc. The combined organic extracts were dried (MgSO4), filtered and the solvents were evaporated in vacuo. The crude product was purified by flash column chromatography (silica; EtOAc in heptane, gradient from 0/100 to 15/85). The desired fractions were collected and concentrated in vacuo to afford a mixture of I-190 and I-191 (270 mg, 51%).

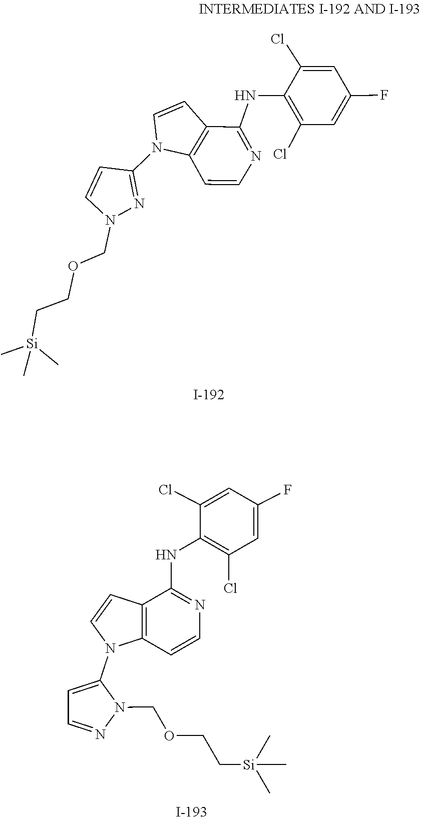 [SMILES: C[Si](C)(C)CCOCn1ccc(-n2ccc3c(Nc4c(Cl)cc(F)cc4Cl)nccc32)n1 ; C[Si](C)(C)CCOCn1nccc1-n1ccc2c(Nc3c(Cl)cc(F)cc3Cl)nccc21]

Pd₂dba₃ (39.1 mg, 42.6 μmol), XantPhos (61.7 mg, 0.11 mmol) and Cs₂CO₃ (521 mg, 1.60 mmol) were added to a solution of I-190 and I-191 (372 mg, 1.07 mmol) in anhydrous DMF (12 mL) in a sealed tube while nitrogen was bubbling. After 10 min, 2,6-dichloro-4-fluoroaniline [344-19-4] (249 mg, 1.39 mmol) was added. The reaction mixture was stirred at room temperature for 10 min, and at 100° C. for 20 h. The mixture was filtered over a pad of Celite® and the filtrate was concentrated in vacuo. The crude product was purified by flash column chromatography (silica; EtOAc in heptane, gradient from 0/100 to 100/0)). The desired fractions were collected and concentrated in vacuo to afford a mixture of I-192 and I-193 (376 mg, 71%).

### Preparation of Final Compounds

### Preparation of Compound 1

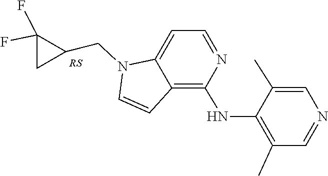 [SMILES: Cc1cncc(C)c1Nc1nccc2c1ccn2CC1CC1(F)F]

To a solution of I-49 (39 mg, 0.16 mmol) dissolved in DMF (1.3 mL) at 0° C. was added sodium hydride (60% dispersion in mineral oil, 7.2 mg, 0.18 mmol) and the reaction mixture was allowed to warm to RT and stirred until gas evolution halted, at which point 2-(bromomethyl)-1,1-difluorocyclopropane [77613-65-1] (33.6 mg, 0.2 mmol) was added at 0° C. Then the reaction mixture was stirred at rt for 16 h. The reaction mixture was then quenched with water and EtOAc was added. The aqueous layer was extracted three times with EtOAc. The combined organic layers were washed with brine, dried (MgSO₄), filtered and concentrated. The residue was then purified by flash column chromatography (silica gel; DCM/7N NH₃ in MeOH, gradient from 100/0 to 98/2) to afford Co. No. 1 (16.7 mg, 31%).

Compound 2 was synthesized in an analogous manner from the indicated intermediate and reagent:

| Starting material | Reagent | Compound |
|---|---|---|
| I-50 | 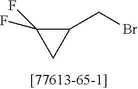 | 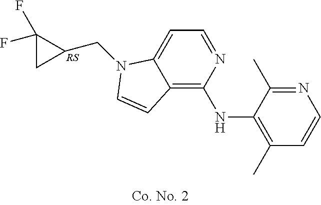 |

### Preparation of Compound 3

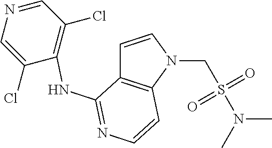 [SMILES: CN(C)S(=O)(=O)Cn1ccc2c(Nc3c(Cl)cncc3Cl)nccc21]

A mixture of I-11 (80 mg, 0.292 mmol), 4-amino-3,5-dichloropyiridine ([22889-78-7], 55.894 mg, 0.343 mmol), and Cs₂CO₃ (209.482 mg, 0.643 mmol) in tBuOH (1.097 mL) was degassed with nitrogen. Pd(OAc)₂ (6.561 mg, 0.0292 mmol) and Xantphos (16.91 mg, 0.0292 mmol) were added and the mixture was heated at 110° C. for 24 h. The solvent was removed in vacuo and then the crude was diluted with water, and extracted with DCM. The combined organic extracts were dried over MgSO₄, filtered and the solvent was removed. The crude was purified by reverse phase chromatography (eluent: MeOH and NH₄CO₃) to obtain Co. No. 3 (26.7 mg, yield 22.8%) as a white powder.

| INTERMEDIATE | ANILINE | COMPOUND |
|---|---|---|
| I-15 | 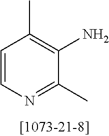 | 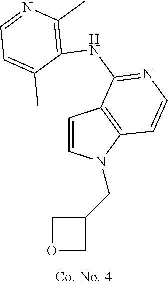 [SMILES: Cc1ccnc(C)c1Nc1nccc2c1ccn2CC1COC1] |
| I-1 | 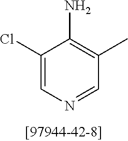 | 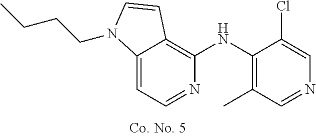 [SMILES: CCCCn1ccc2c(Nc3c(C)cncc3Cl)nccc21] |
| I-1 | 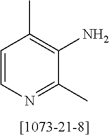 | 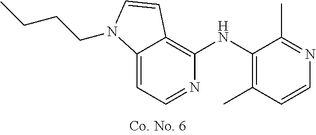 [SMILES: CCCCn1ccc2c(Nc3c(C)ccnc3C)nccc21] |
| I-3 | 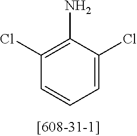 | 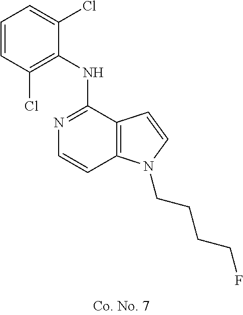 [SMILES: FCCCCn1ccc2c(Nc3c(Cl)cccc3Cl)nccc21] |
| I-1 | 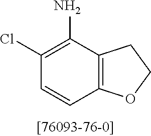 [SMILES: Nc1c(Cl)ccc2c1CCO2] | 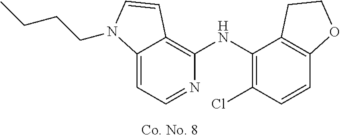 [SMILES: CCCCn1ccc2c(Nc3c(Cl)ccc4c3CCO4)nccc21] |
| I-27 | 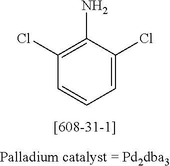 | 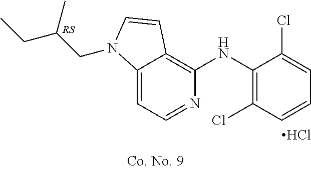 |
| I-2 | 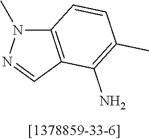 | 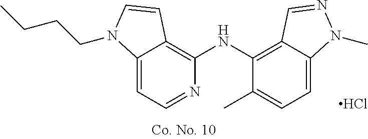 [SMILES: CCCCn1ccc2c(Nc3c(C)ccc4c3cnn4C)nccc21 ; Cl] |
| I-2 | 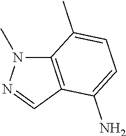 | 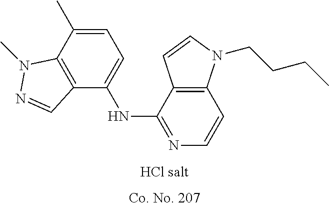 |
| I-2 | 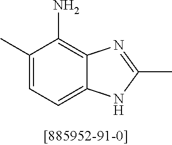 | 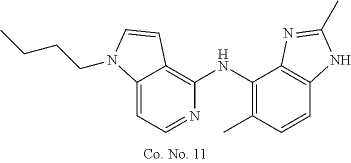 [SMILES: CCCCn1ccc2c(Nc3c(C)ccc4[nH]c(C)nc34)nccc21] |
| I-2 | 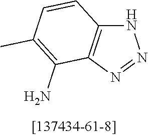 | 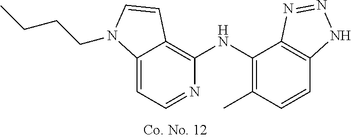 [SMILES: CCCCn1ccc2c(Nc3c(C)ccc4[nH]nnc34)nccc21] |
| I-1 | 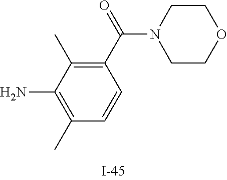 [SMILES: Cc1ccc(C(=O)N2CCOCC2)c(C)c1N] | 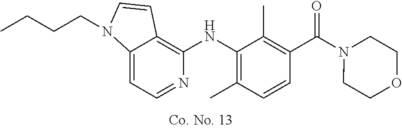 [SMILES: CCCCn1ccc2c(Nc3c(C)ccc(C(=O)N4CCOCC4)c3C)nccc21] |
| I-1 | 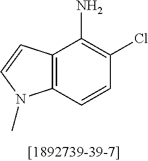 | 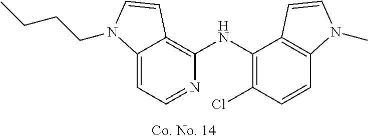 [SMILES: CCCCn1ccc2c(Nc3c(Cl)ccc4c3ccn4C)nccc21] |
| I-1 | 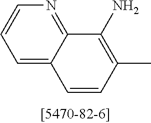 | 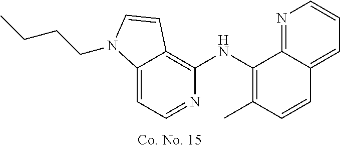 [SMILES: CCCCn1ccc2c(Nc3c(C)ccc4cccnc34)nccc21] |
| I-27 | 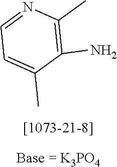 | 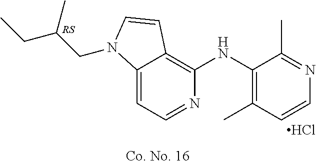 |
| I-1 | 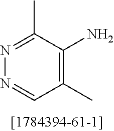 | 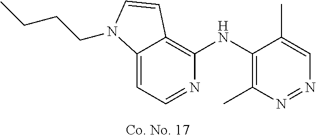 [SMILES: CCCCn1ccc2c(Nc3c(C)cnnc3C)nccc21] |
| I-2 | I-51 | 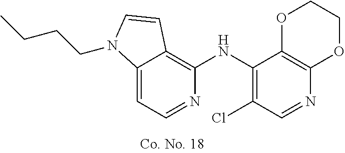 [SMILES: CCCCn1ccc2c(Nc3c(Cl)cnc4c3OCCO4)nccc21] |
| I-2 | I-54 | 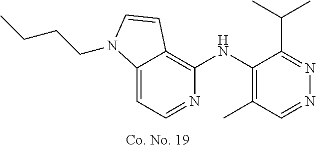 [SMILES: CCCCn1ccc2c(Nc3c(C)cnnc3C(C)C)nccc21] |
| I-2 | I-56 | 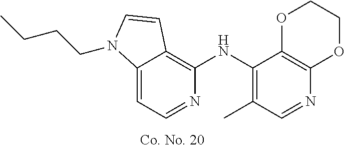 [SMILES: CCCCn1ccc2c(Nc3c(C)cnc4c3OCCO4)nccc21] |
| I-23 | 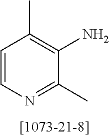 | 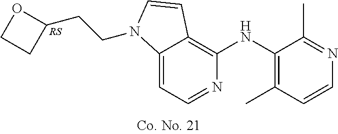 |
| I-4 | 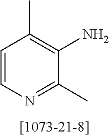 | 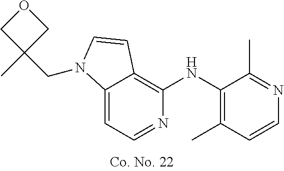 [SMILES: Cc1ccnc(C)c1Nc1nccc2c1ccn2CC1(C)COC1] |
| I-28 | 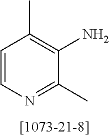 | 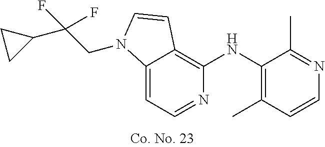 [SMILES: Cc1ccnc(C)c1Nc1nccc2c1ccn2CC(F)(F)C1CC1] |
| I-28 | 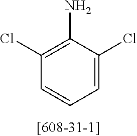 | 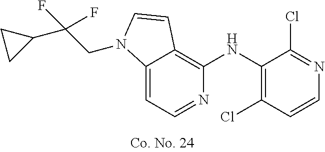 [SMILES: FC(F)(Cn1ccc2c(Nc3c(Cl)ccnc3Cl)nccc21)C1CC1] |
| I-15 | 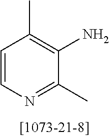 | 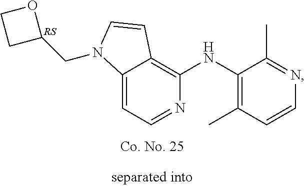 |
| | | 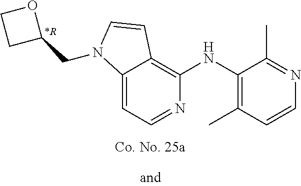 |
| | | 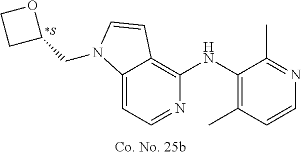 |
| I-8b | 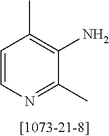 | 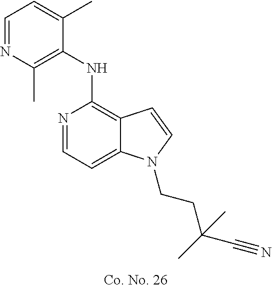 [SMILES: Cc1ccnc(C)c1Nc1nccc2c1ccn2CCC(C)(C)C#N] |
| I-9 | 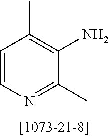 | 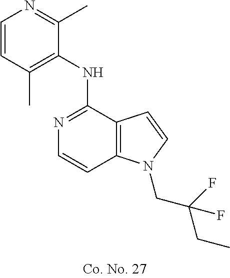 [SMILES: CCC(F)(F)Cn1ccc2c(Nc3c(C)ccnc3C)nccc21] |
| I-13 | 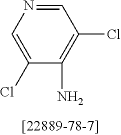 | 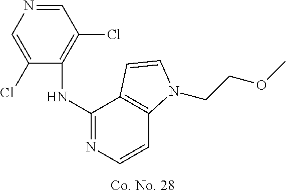 [SMILES: COCCn1ccc2c(Nc3c(Cl)cncc3Cl)nccc21] |
| I-15 | 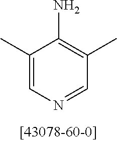 | 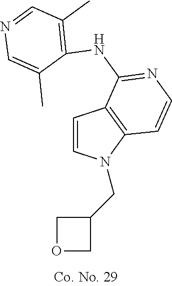 [SMILES: Cc1cncc(C)c1Nc1nccc2c1ccn2CC1COC1] |
| I-15 | 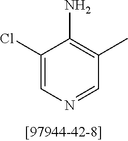 | 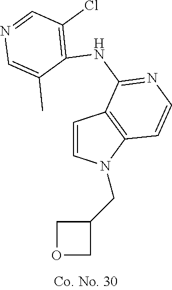 [SMILES: Cc1cncc(Cl)c1Nc1nccc2c1ccn2CC1COC1] |
| I-57 | 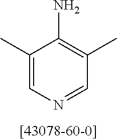 | 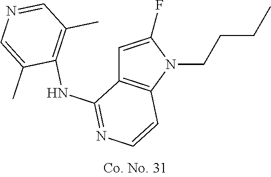 [SMILES: CCCCn1c(F)cc2c(Nc3c(C)cncc3C)nccc21] |
| I-15 | I-40a | 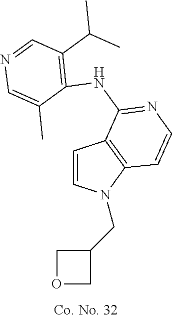 [SMILES: Cc1cncc(C(C)C)c1Nc1nccc2c1ccn2CC1COC1] |
| I-17 | 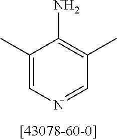 | 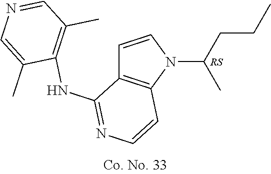 |
| I-58 | 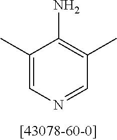 | 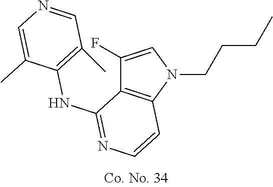 [SMILES: CCCCn1cc(F)c2c(Nc3c(C)cncc3C)nccc21] |
| I-15 | 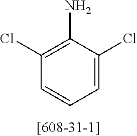 | 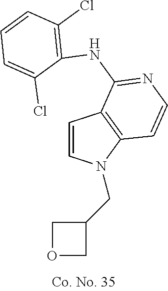 [SMILES: Clc1cccc(Cl)c1Nc1nccc2c1ccn2CC1COC1] |
| I-12 | 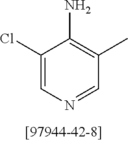 | 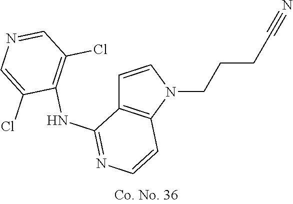 [SMILES: N#CCCCn1ccc2c(Nc3c(Cl)cncc3Cl)nccc21] |
| I-19 | 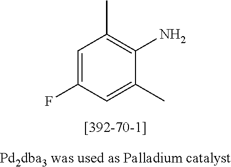 | 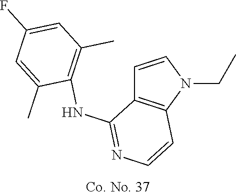 [SMILES: CCn1ccc2c(Nc3c(C)cc(F)cc3C)nccc21] |
| I-18b | 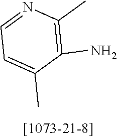 | 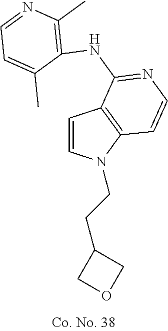 [SMILES: Cc1ccnc(C)c1Nc1nccc2c1ccn2CCC1COC1] |
| I-17 | 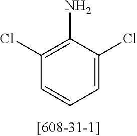 | 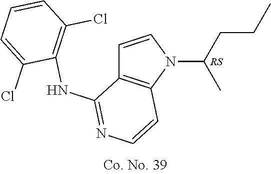 |
| I-13 | 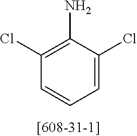 | 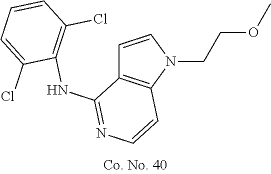 [SMILES: COCCn1ccc2c(Nc3c(Cl)cccc3Cl)nccc21] |
| I-6b | 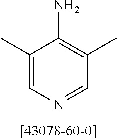 | 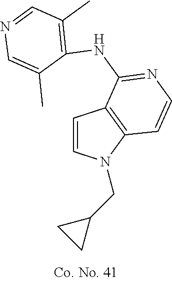 [SMILES: Cc1cncc(C)c1Nc1nccc2c1ccn2CC1CC1] |
| I-1 | 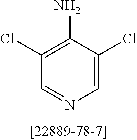 | 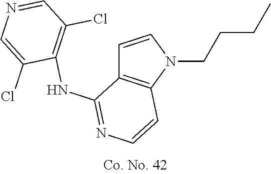 [SMILES: CCCCn1ccc2c(Nc3c(Cl)cncc3Cl)nccc21] |
| I-1 | 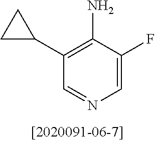 [SMILES: Nc1c(F)cncc1C1CC1] | 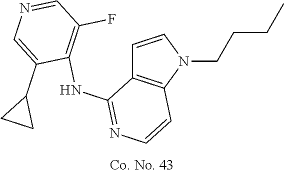 [SMILES: CCCCn1ccc2c(Nc3c(F)cncc3C3CC3)nccc21] |
| I-1 | 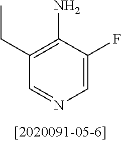 | 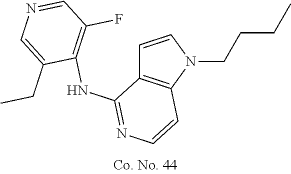 [SMILES: CCCCn1ccc2c(Nc3c(F)cncc3CC)nccc21] |
| I-2 | I-37 | 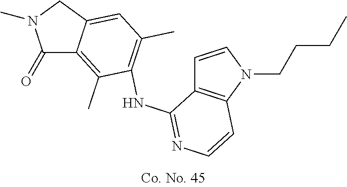 [SMILES: CCCCn1ccc2c(Nc3c(C)cc4c(c3C)C(=O)N(C)C4)nccc21] |
| I-1 | I-40 | 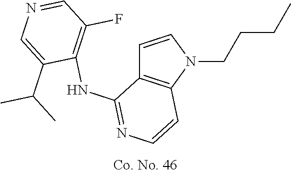 [SMILES: CCCCn1ccc2c(Nc3c(F)cncc3C(C)C)nccc21] |
| I-16 | 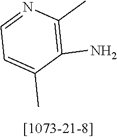 | 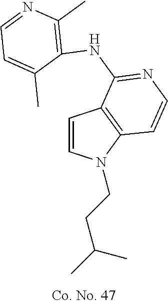 [SMILES: Cc1ccnc(C)c1Nc1nccc2c1ccn2CCC(C)C] |
| I-16 | 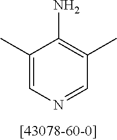 | 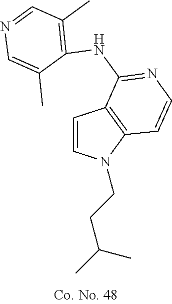 [SMILES: Cc1cncc(C)c1Nc1nccc2c1ccn2CCC(C)C] |
| I-7b | 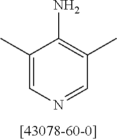 | 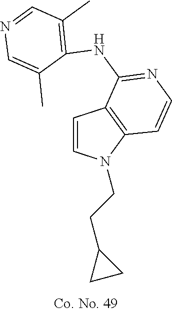 [SMILES: Cc1cncc(C)c1Nc1nccc2c1ccn2CCC1CC1] |
| I-12 | 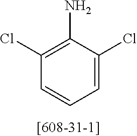 | 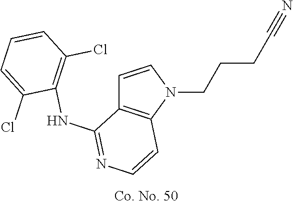 [SMILES: N#CCCCn1ccc2c(Nc3c(Cl)cccc3Cl)nccc21] |
| I-1 | 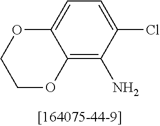 [SMILES: Nc1c(Cl)ccc2c1OCCO2] | 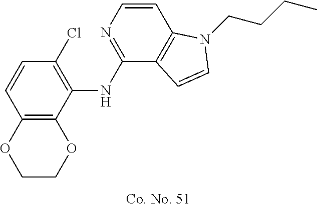 [SMILES: CCCCn1ccc2c(Nc3c(Cl)ccc4c3OCCO4)nccc21] |
| I-1 | 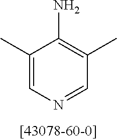 | 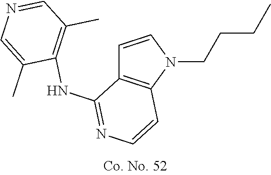 [SMILES: CCCCn1ccc2c(Nc3c(C)cncc3C)nccc21] |
| I-2 | I-42 | 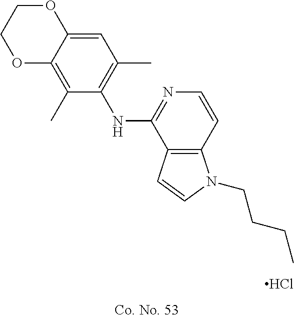 [SMILES: CCCCn1ccc2c(Nc3c(C)cc4c(c3C)OCCO4)nccc21 ; Cl] |
| I-2 | I-43 | 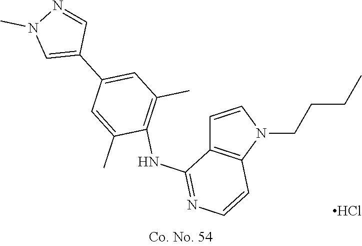 [SMILES: CCCCn1ccc2c(Nc3c(C)cc(-c4cnn(C)c4)cc3C)nccc21 ; Cl] |
| I-1 | I-44 | 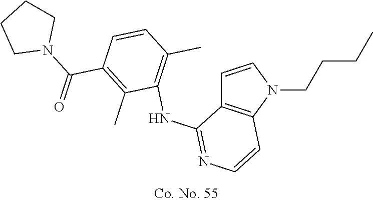 [SMILES: CCCCn1ccc2c(Nc3c(C)ccc(C(=O)N4CCCC4)c3C)nccc21] |
| I-1 | I-45 | 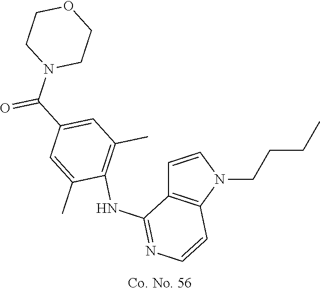 [SMILES: CCCCn1ccc2c(Nc3c(C)cc(C(=O)N4CCOCC4)cc3C)nccc21] |
| I-1 | I-46 | 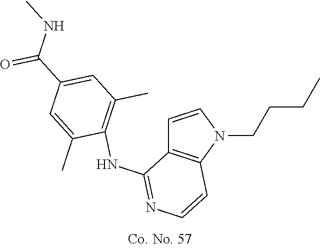 [SMILES: CCCCn1ccc2c(Nc3c(C)cc(C(=O)NC)cc3C)nccc21] |
| I-1 | I-47 | 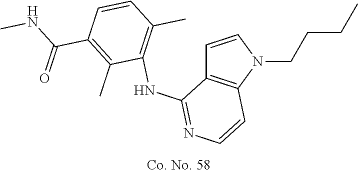 [SMILES: CCCCn1ccc2c(Nc3c(C)ccc(C(=O)NC)c3C)nccc21] |
| I-1 | I-48 | 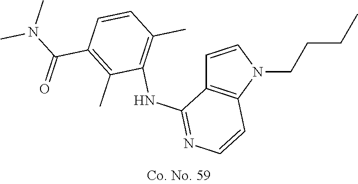 [SMILES: CCCCn1ccc2c(Nc3c(C)ccc(C(=O)N(C)C)c3C)nccc21] |
| I-16 | 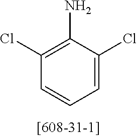 | 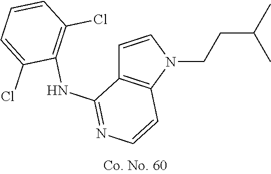 [SMILES: CC(C)CCn1ccc2c(Nc3c(Cl)cccc3Cl)nccc21] |
| I-1 | 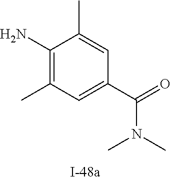 | 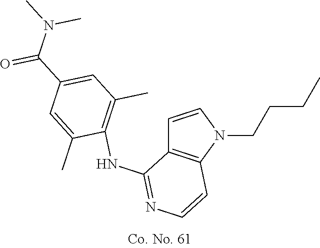 [SMILES: CCCCn1ccc2c(Nc3c(C)cc(C(=O)N(C)C)cc3C)nccc21] |
| I-1 | 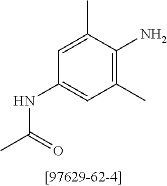 | 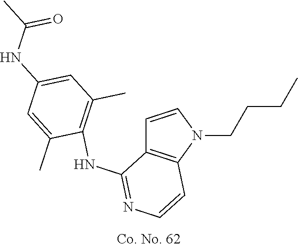 [SMILES: CCCCn1ccc2c(Nc3c(C)cc(NC(C)=O)cc3C)nccc21] |
| I-2 | I-40a | 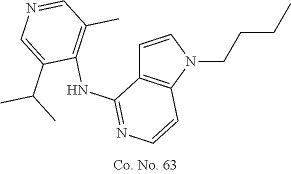 [SMILES: CCCCn1ccc2c(Nc3c(C)cncc3C(C)C)nccc21] |
| I-2 | 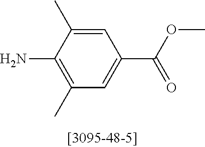 | 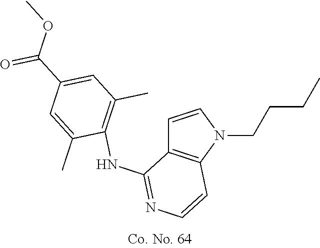 [SMILES: CCCCn1ccc2c(Nc3c(C)cc(C(=O)OC)cc3C)nccc21] |
| I-7b | 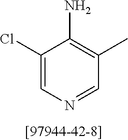 | 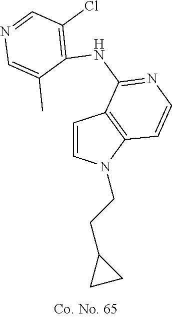 [SMILES: Cc1cncc(Cl)c1Nc1nccc2c1ccn2CCC1CC1] |
| I-2 | 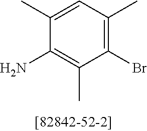 | 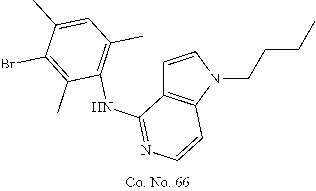 [SMILES: CCCCn1ccc2c(Nc3c(C)cc(C)c(Br)c3C)nccc21] |
| I-1 | 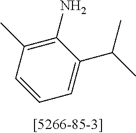 | 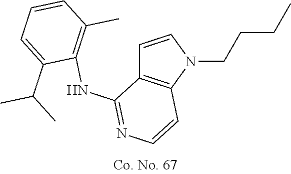 [SMILES: CCCCn1ccc2c(Nc3c(C)cccc3C(C)C)nccc21] |
| I-1 | 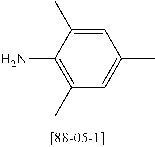 | 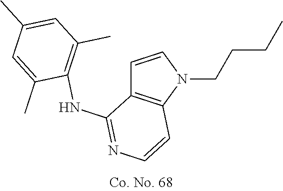 [SMILES: CCCCn1ccc2c(Nc3c(C)cc(C)cc3C)nccc21] |
| I-6a | 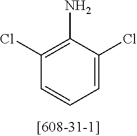 | 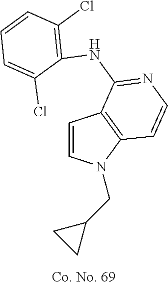 [SMILES: Clc1cccc(Cl)c1Nc1nccc2c1ccn2CC1CC1] |
| 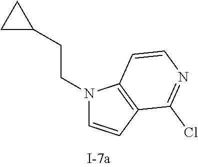 [SMILES: Clc1nccc2c1ccn2CCC1CC1] | 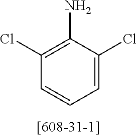 | 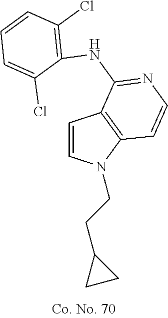 [SMILES: Clc1cccc(Cl)c1Nc1nccc2c1ccn2CCC1CC1] |
| I-1 | 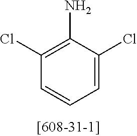 | 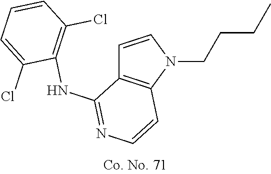 [SMILES: CCCCn1ccc2c(Nc3c(Cl)cccc3Cl)nccc21] |
| I-1 | 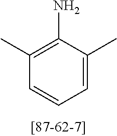 | 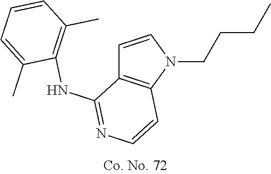 [SMILES: CCCCn1ccc2c(Nc3c(C)cccc3C)nccc21] |
| I-2 | 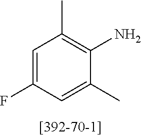 | 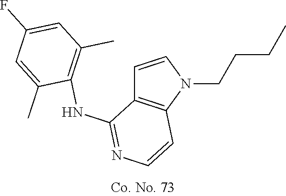 [SMILES: CCCCn1ccc2c(Nc3c(C)cc(F)cc3C)nccc21] |
| I-26a | 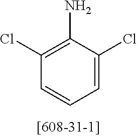 | 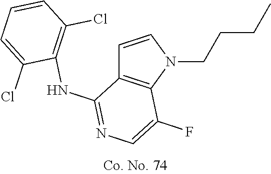 [SMILES: CCCCn1ccc2c(Nc3c(Cl)cccc3Cl)ncc(F)c21] |
| I-1 | 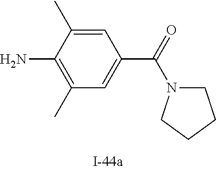 [SMILES: Cc1cc(C(=O)N2CCCC2)cc(C)c1N] | 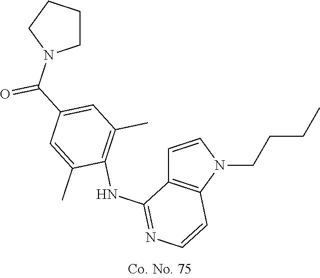 [SMILES: CCCCn1ccc2c(Nc3c(C)cc(C(=O)N4CCCC4)cc3C)nccc21] |
| I-2 | 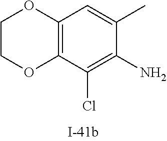 | 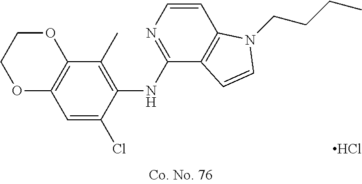 [SMILES: CCCCn1ccc2c(Nc3c(Cl)cc4c(c3C)OCCO4)nccc21 ; Cl] |
| I-2 | 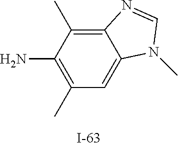 | 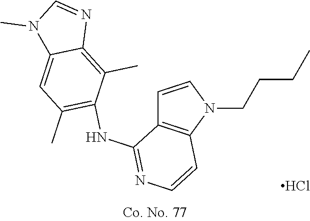 [SMILES: CCCCn1ccc2c(Nc3c(C)cc4c(ncn4C)c3C)nccc21 ; Cl] |
| I-30 | 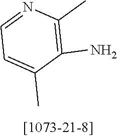 | 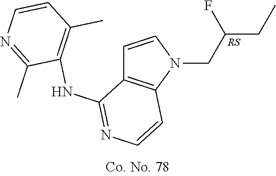 |
| I-10 | 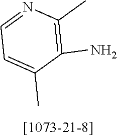 | 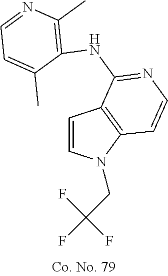 [SMILES: Cc1ccnc(C)c1Nc1nccc2c1ccn2CC(F)(F)F] |
| I-26a | 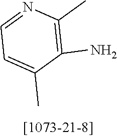 | 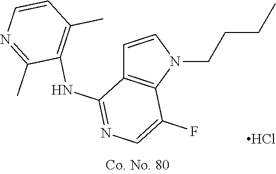 [SMILES: CCCCn1ccc2c(Nc3c(C)ccnc3C)ncc(F)c21 ; Cl] |
| I-5b | 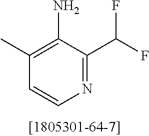 | 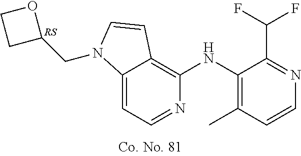 |
| I-5b | 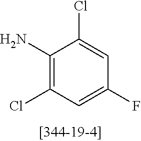 | 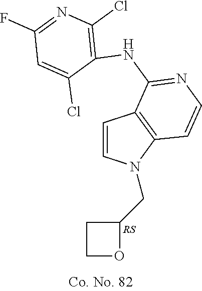 |
| I-8a | 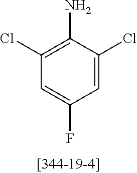 | 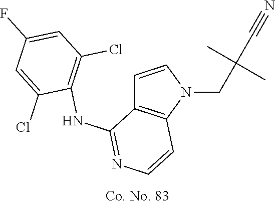 [SMILES: CC(C)(C#N)Cn1ccc2c(Nc3c(Cl)cc(F)cc3Cl)nccc21] |
| I-8a | 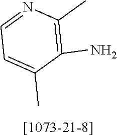 | 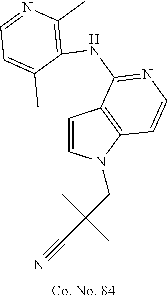 [SMILES: Cc1ccnc(C)c1Nc1nccc2c1ccn2CC(C)(C)C#N] |
| I-28 | 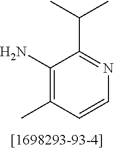 | 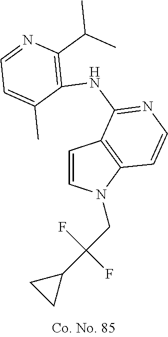 [SMILES: Cc1ccnc(C(C)C)c1Nc1nccc2c1ccn2CC(F)(F)C1CC1] |
| I-65 | 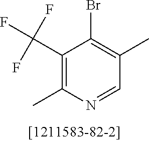 | 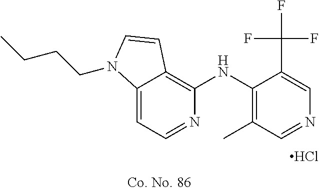 [SMILES: CCCCn1ccc2c(Nc3c(C)cncc3C(F)(F)F)nccc21 ; Cl] |
| I-2 | 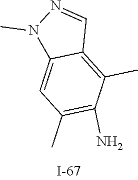 | 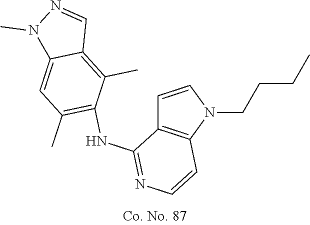 [SMILES: CCCCn1ccc2c(Nc3c(C)cc4c(cnn4C)c3C)nccc21] |
| I-2 | I-71 | 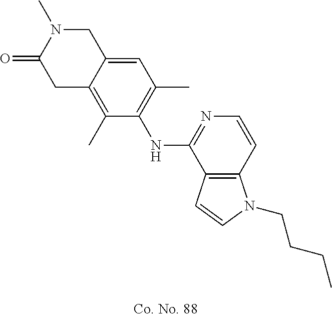 [SMILES: CCCCn1ccc2c(Nc3c(C)cc4c(c3C)CC(=O)N(C)C4)nccc21] |
| I-5b | 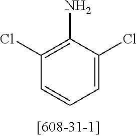 | 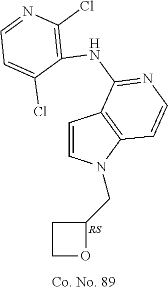 |
| I-29 | 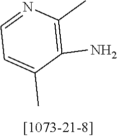 | 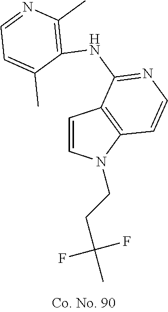 [SMILES: Cc1ccnc(C)c1Nc1nccc2c1ccn2CCC(C)(F)F] |
| I-24 | 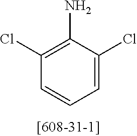 | 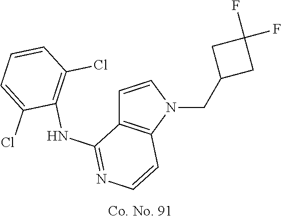 [SMILES: FC1(F)CC(Cn2ccc3c(Nc4c(Cl)cccc4Cl)nccc32)C1] |
| I-24 | 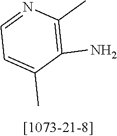 | 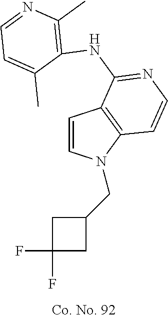 [SMILES: Cc1ccnc(C)c1Nc1nccc2c1ccn2CC1CC(F)(F)C1] |
| I-22 | 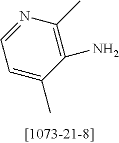 | 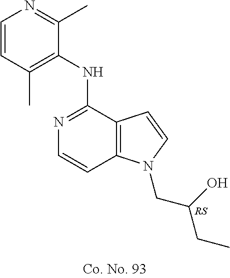 |
| I-25 | 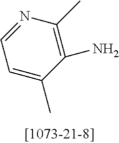 | 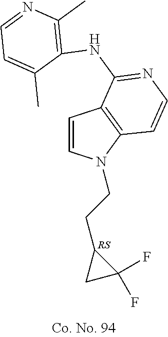 |
| I-25 | 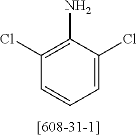 | 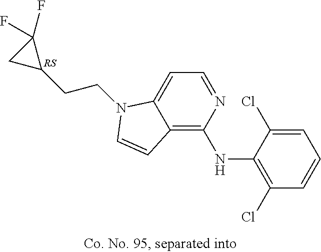 |
| | | 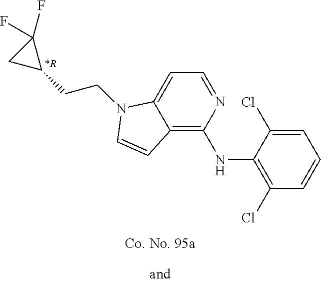 |
| | | 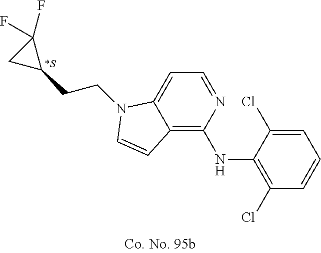 |
| I-33 | 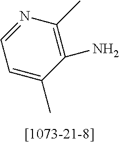 | 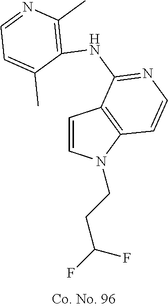 [SMILES: Cc1ccnc(C)c1Nc1nccc2c1ccn2CCC(F)F] |
| I-33 | 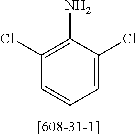 | 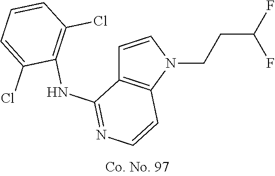 [SMILES: FC(F)CCn1ccc2c(Nc3c(Cl)cccc3Cl)nccc21] |
| I-18a | 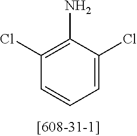 | 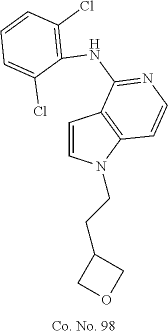 [SMILES: Clc1cccc(Cl)c1Nc1nccc2c1ccn2CCC1COC1] |
| I-18a | 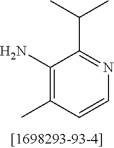 | 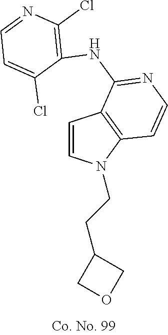 [SMILES: Clc1ccnc(Cl)c1Nc1nccc2c1ccn2CCC1COC1] |
| I-26b | 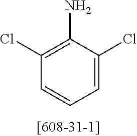 | 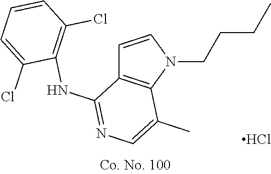 [SMILES: CCCCn1ccc2c(Nc3c(Cl)cccc3Cl)ncc(C)c21 ; Cl] |
| 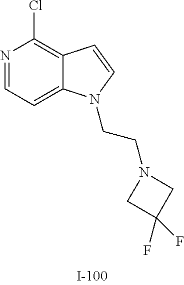 [SMILES: FC1(F)CN(CCn2ccc3c(Cl)nccc32)C1] | 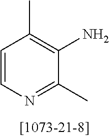 | 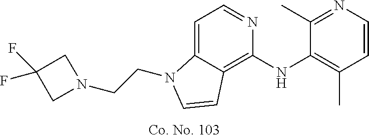 [SMILES: Cc1ccnc(C)c1Nc1nccc2c1ccn2CCN1CC(F)(F)C1] |
| 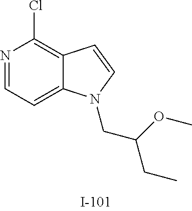 [SMILES: CCC(Cn1ccc2c(Cl)nccc21)OC] | 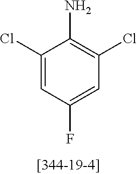 | 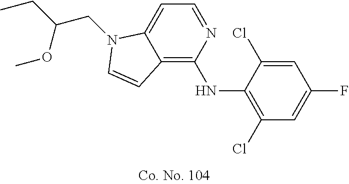 [SMILES: CCC(Cn1ccc2c(Nc3c(Cl)cc(F)cc3Cl)nccc21)OC] |
| 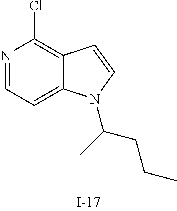 | 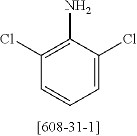 | 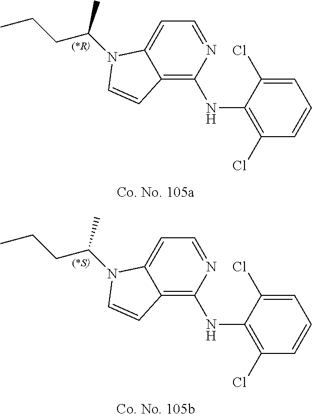 |
| 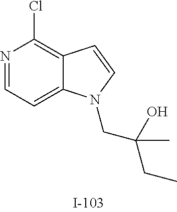 [SMILES: CCC(C)(O)Cn1ccc2c(Cl)nccc21] | 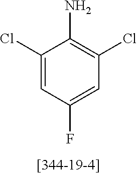 | 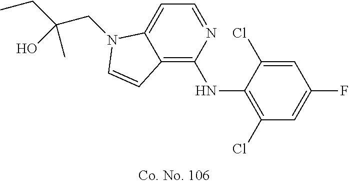 [SMILES: CCC(C)(O)Cn1ccc2c(Nc3c(Cl)cc(F)cc3Cl)nccc21] |
| 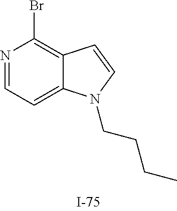 | 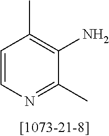 | 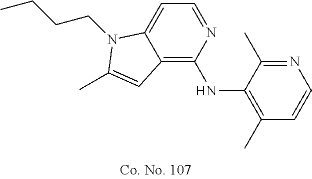 [SMILES: CCCCn1c(C)cc2c(Nc3c(C)ccnc3C)nccc21] |
| 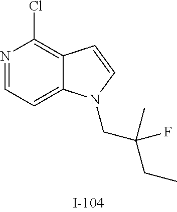 [SMILES: CCC(C)(F)Cn1ccc2c(Cl)nccc21] | 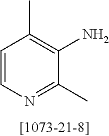 | 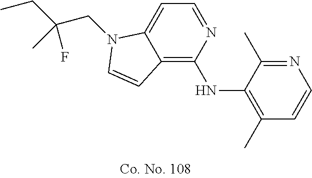 [SMILES: CCC(C)(F)Cn1ccc2c(Nc3c(C)ccnc3C)nccc21] |
| 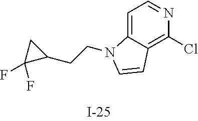 [SMILES: FC1(F)CC1CCn1ccc2c(Cl)nccc21] | 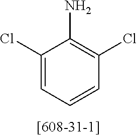 | 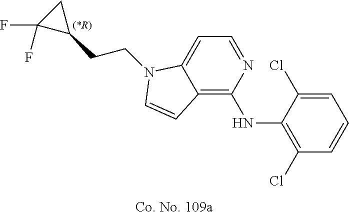 |
| | | 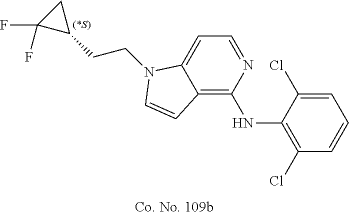 |
| 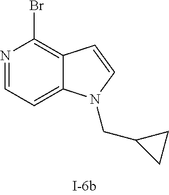 [SMILES: Brc1nccc2c1ccn2CC1CC1] | 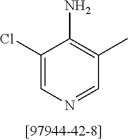 | 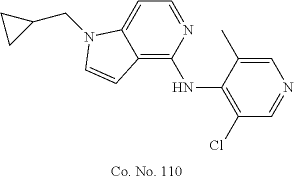 [SMILES: Cc1cncc(Cl)c1Nc1nccc2c1ccn2CC1CC1] |
| 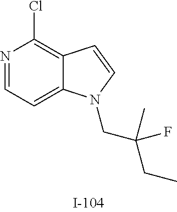 [SMILES: CCC(C)(F)Cn1ccc2c(Cl)nccc21] | 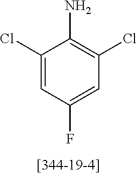 | 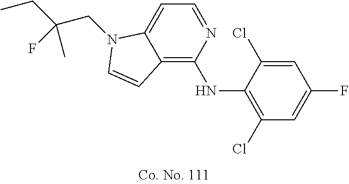 [SMILES: CCC(C)(F)Cn1ccc2c(Nc3c(Cl)cc(F)cc3Cl)nccc21] |
| 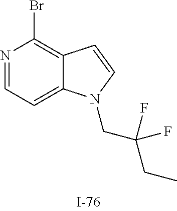 [SMILES: CCC(F)(F)Cn1ccc2c(Br)nccc21] | 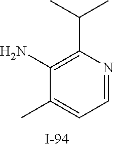 | 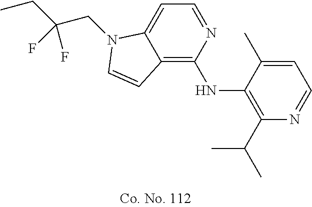 [SMILES: CCC(F)(F)Cn1ccc2c(Nc3c(C)ccnc3C(C)C)nccc21] |
| 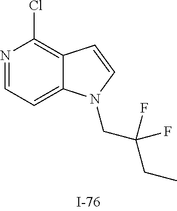 [SMILES: CCC(F)(F)Cn1ccc2c(Cl)nccc21] | 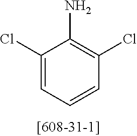 | 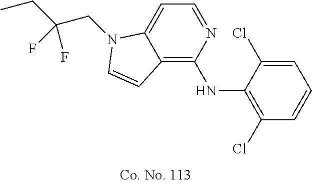 [SMILES: CCC(F)(F)Cn1ccc2c(Nc3c(Cl)cccc3Cl)nccc21] |
| 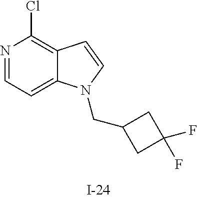 [SMILES: FC1(F)CC(Cn2ccc3c(Cl)nccc32)C1] | 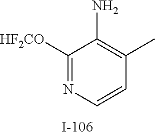 | 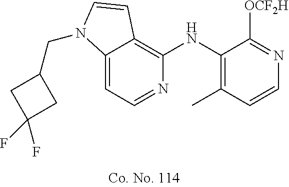 |
| 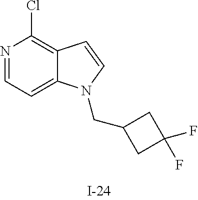 [SMILES: FC1(F)CC(Cn2ccc3c(Cl)nccc32)C1] | 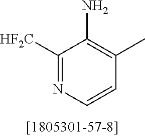 | 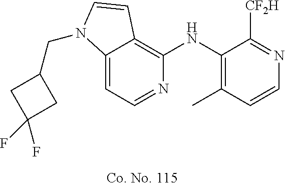 [SMILES: Cc1ccnc(C(F)F)c1Nc1nccc2c1ccn2CC1CC(F)(F)C1] |
| 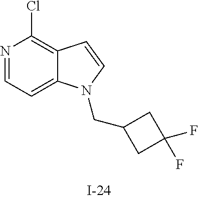 [SMILES: FC1(F)CC(Cn2ccc3c(Cl)nccc32)C1] | 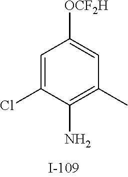 | 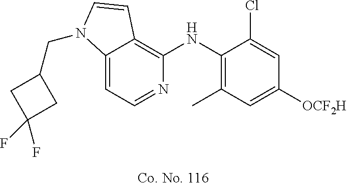 |
| 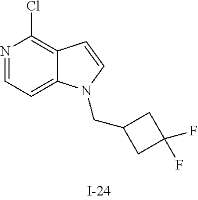 [SMILES: FC1(F)CC(Cn2ccc3c(Cl)nccc32)C1] | 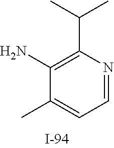 | 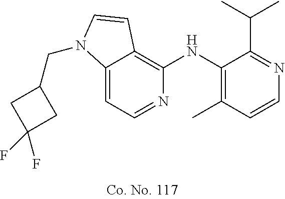 [SMILES: Cc1ccnc(C(C)C)c1Nc1nccc2c1ccn2CC1CC(F)(F)C1] |
| 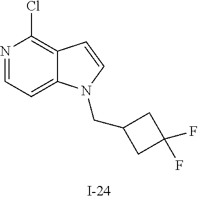 [SMILES: FC1(F)CC(Cn2ccc3c(Cl)nccc32)C1] | 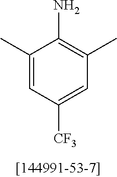 | 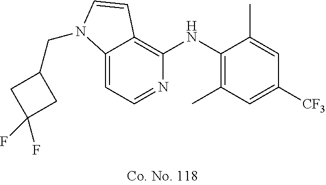 [SMILES: Cc1cc(C(F)(F)F)cc(C)c1Nc1nccc2c1ccn2CC1CC(F)(F)C1] |
| 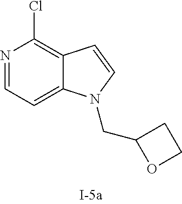 [SMILES: Clc1nccc2c1ccn2CC1CCO1] | 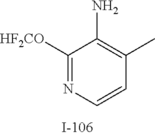 | 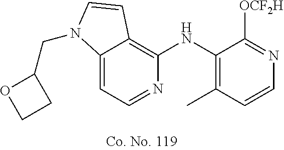 |
| 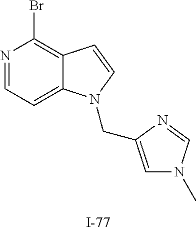 [SMILES: Cn1cnc(Cn2ccc3c(Br)nccc32)c1] | 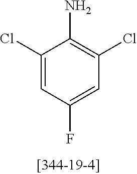 | 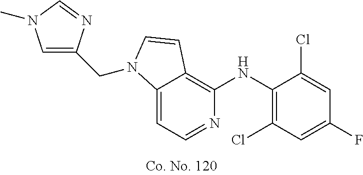 [SMILES: Cn1cnc(Cn2ccc3c(Nc4c(Cl)cc(F)cc4Cl)nccc32)c1] |
| 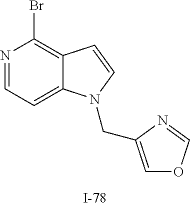 [SMILES: Brc1nccc2c1ccn2Cc1cocn1] | 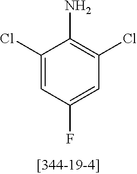 | 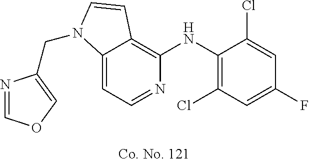 [SMILES: Fc1cc(Cl)c(Nc2nccc3c2ccn3Cc2cocn2)c(Cl)c1] |
| 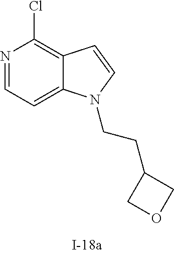 [SMILES: Clc1nccc2c1ccn2CCC1COC1] | 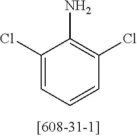 | 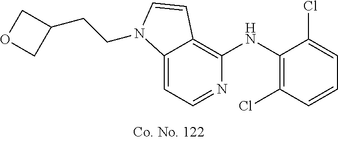 [SMILES: Clc1cccc(Cl)c1Nc1nccc2c1ccn2CCC1COC1] |
| 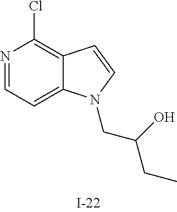 | 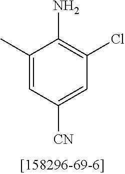 | 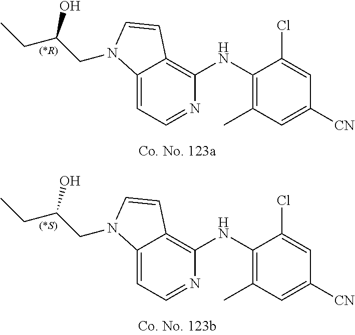 |
| 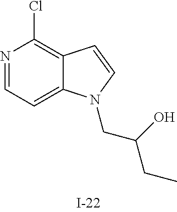 | 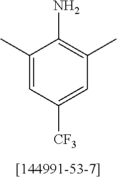 | 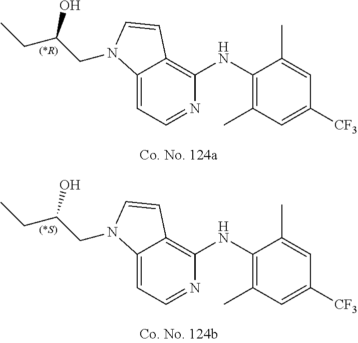 |
| 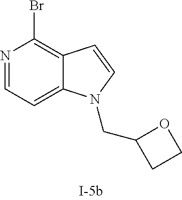 [SMILES: Brc1nccc2c1ccn2CC1CCO1] | 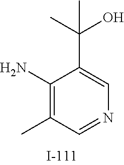 | 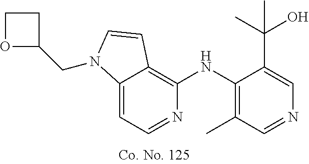 [SMILES: Cc1cncc(C(C)(C)O)c1Nc1nccc2c1ccn2CC1CCO1] |
| 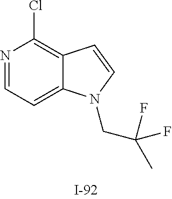 | 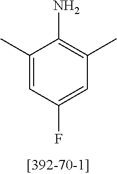 | 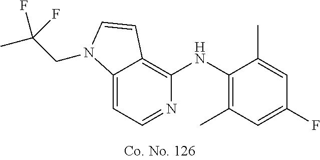 [SMILES: Cc1cc(F)cc(C)c1Nc1nccc2c1ccn2CC(C)(F)F] |
| 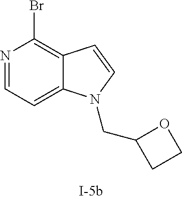 [SMILES: Brc1nccc2c1ccn2CC1CCO1] | 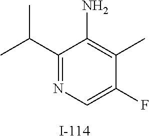 | 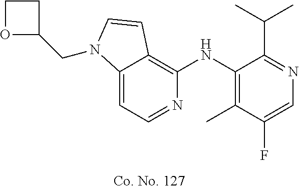 [SMILES: Cc1c(F)cnc(C(C)C)c1Nc1nccc2c1ccn2CC1CCO1] |
| 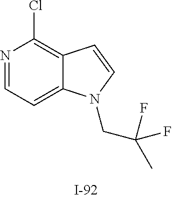 | 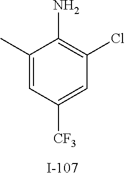 | 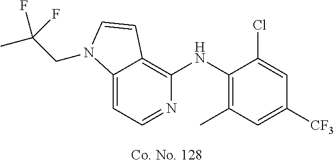 [SMILES: Cc1cc(C(F)(F)F)cc(Cl)c1Nc1nccc2c1ccn2CC(C)(F)F] |
| 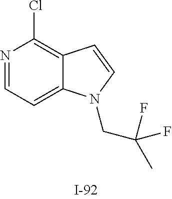 | 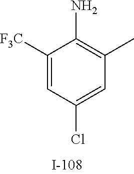 | 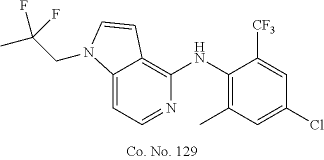 [SMILES: Cc1cc(Cl)cc(C(F)(F)F)c1Nc1nccc2c1ccn2CC(C)(F)F] |
| 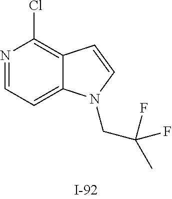 | 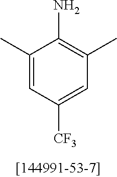 | 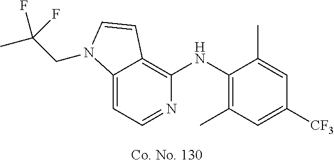 [SMILES: Cc1cc(C(F)(F)F)cc(C)c1Nc1nccc2c1ccn2CC(C)(F)F] |
| 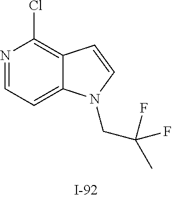 | 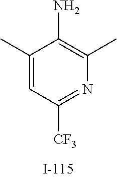 | 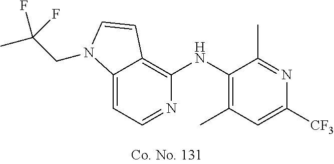 [SMILES: Cc1cc(C(F)(F)F)nc(C)c1Nc1nccc2c1ccn2CC(C)(F)F] |
| 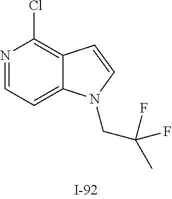 | 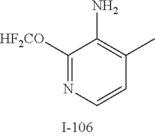 | 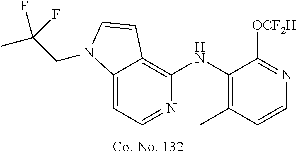 |
| 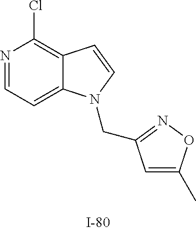 [SMILES: Cc1cc(Cn2ccc3c(Cl)nccc32)no1] | 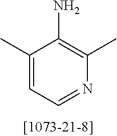 | 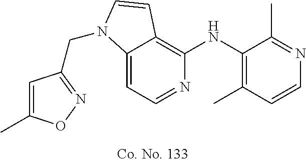 [SMILES: Cc1cc(Cn2ccc3c(Nc4c(C)ccnc4C)nccc32)no1] |
| 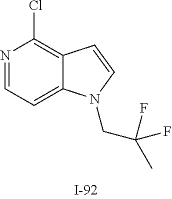 | 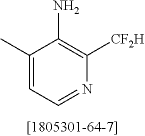 | 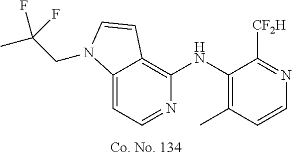 [SMILES: Cc1ccnc(C(F)F)c1Nc1nccc2c1ccn2CC(C)(F)F] |
| 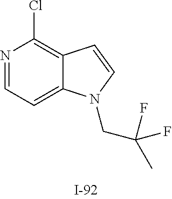 | 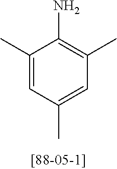 | 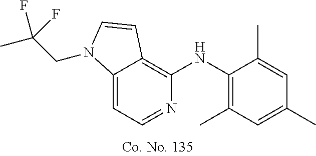 [SMILES: Cc1cc(C)c(Nc2nccc3c2ccn3CC(C)(F)F)c(C)c1] |
| 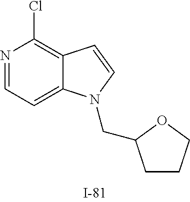 [SMILES: Clc1nccc2c1ccn2CC1CCCO1] | 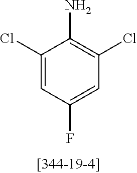 | 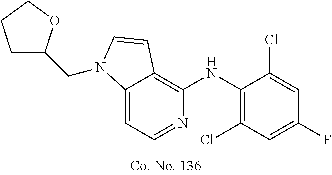 [SMILES: Fc1cc(Cl)c(Nc2nccc3c2ccn3CC2CCCO2)c(Cl)c1] |
| 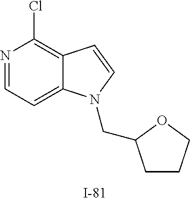 [SMILES: Clc1nccc2c1ccn2CC1CCCO1] | 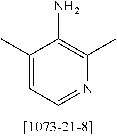 | 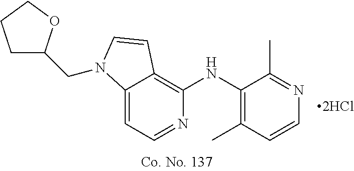 [SMILES: Cc1ccnc(C)c1Nc1nccc2c1ccn2CC1CCCO1 ; "·2HCl"] |
| 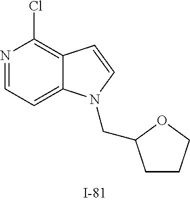 [SMILES: Clc1nccc2c1ccn2CC1CCCO1] | 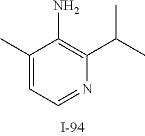 | 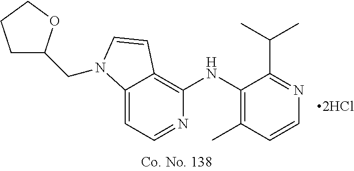 [SMILES: Cc1ccnc(C(C)C)c1Nc1nccc2c1ccn2CC1CCCO1 ; "·2HCl"] |
| 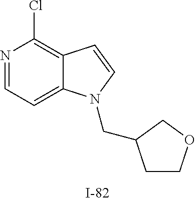 [SMILES: Clc1nccc2c1ccn2CC1CCOC1] | 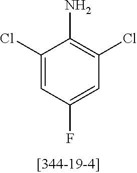 | 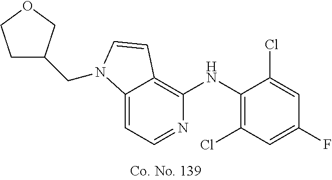 [SMILES: Fc1cc(Cl)c(Nc2nccc3c2ccn3CC2CCOC2)c(Cl)c1] |
| 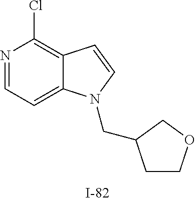 [SMILES: Clc1nccc2c1ccn2CC1CCOC1] | 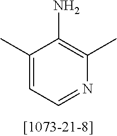 | 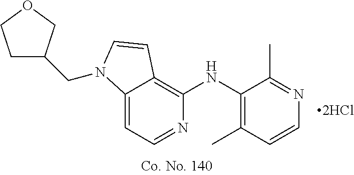 [SMILES: Cc1ccnc(C)c1Nc1nccc2c1ccn2CC1CCOC1 ; "·2HCl"] |
| 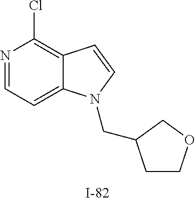 [SMILES: Clc1nccc2c1ccn2CC1CCOC1] | 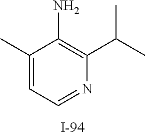 | 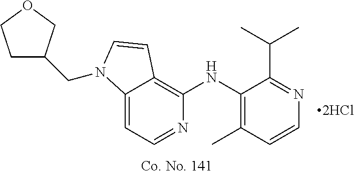 [SMILES: Cc1ccnc(C(C)C)c1Nc1nccc2c1ccn2CC1CCOC1 ; "·2HCl"] |
| 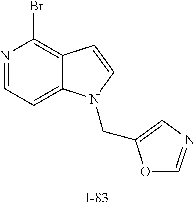 [SMILES: Brc1nccc2c1ccn2Cc1cnco1] | 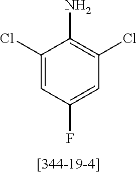 | 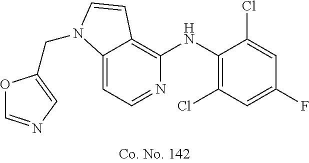 [SMILES: Fc1cc(Cl)c(Nc2nccc3c2ccn3Cc2cnco2)c(Cl)c1] |
| 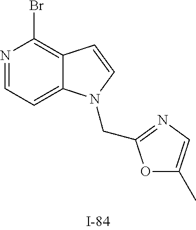 [SMILES: Cc1cnc(Cn2ccc3c(Br)nccc32)o1] | 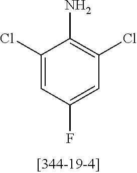 | 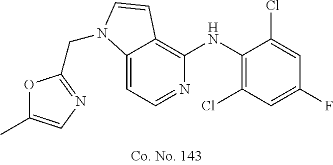 [SMILES: Cc1cnc(Cn2ccc3c(Nc4c(Cl)cc(F)cc4Cl)nccc32)o1] |
| 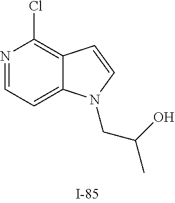 | 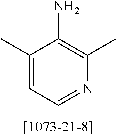 | 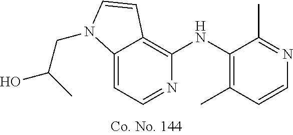 [SMILES: Cc1ccnc(C)c1Nc1nccc2c1ccn2CC(C)O] |
| 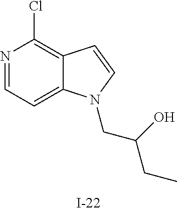 | 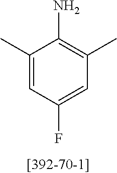 | 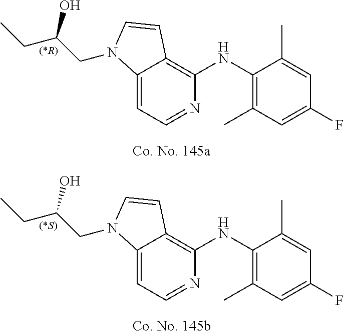 |
| 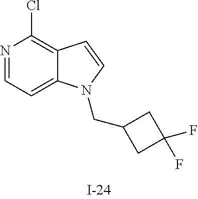 [SMILES: FC1(F)CC(Cn2ccc3c(Cl)nccc32)C1] | 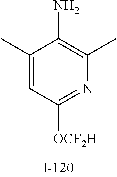 | 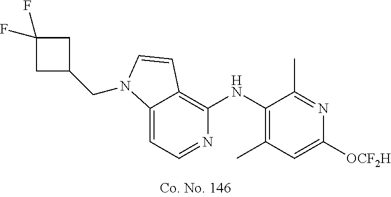 |
| 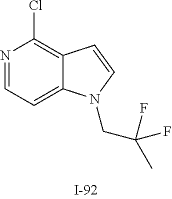 | 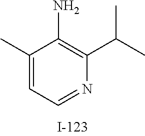 | 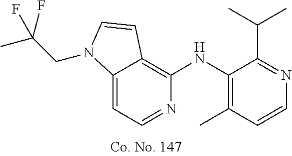 [SMILES: Cc1ccnc(C(C)C)c1Nc1nccc2c1ccn2CC(C)(F)F] |
| 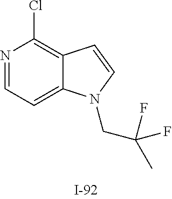 | 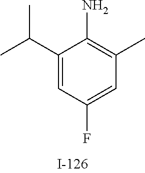 | 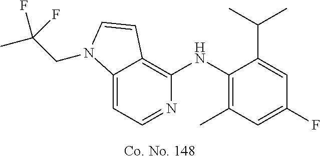 [SMILES: Cc1cc(F)cc(C(C)C)c1Nc1nccc2c1ccn2CC(C)(F)F] |
| 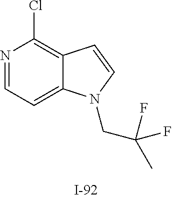 | 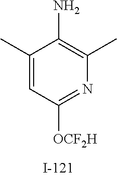 | 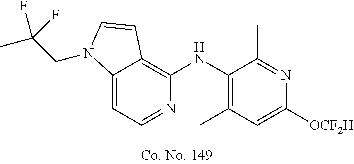 |
| 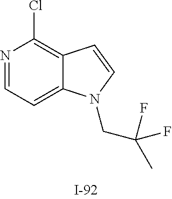 | 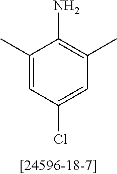 | 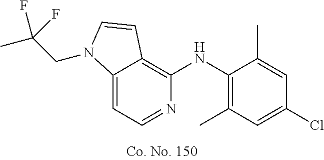 [SMILES: Cc1cc(Cl)cc(C)c1Nc1nccc2c1ccn2CC(C)(F)F] |
| 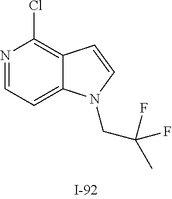 | 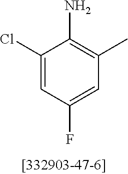 | 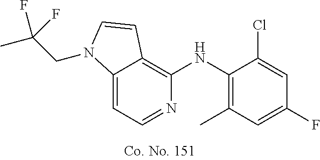 [SMILES: Cc1cc(F)cc(Cl)c1Nc1nccc2c1ccn2CC(C)(F)F] |
| 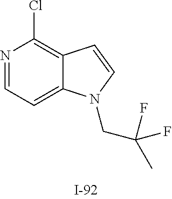 | 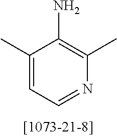 | 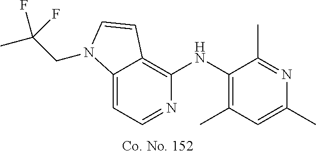 [SMILES: Cc1cc(C)c(Nc2nccc3c2ccn3CC(C)(F)F)c(C)n1] |
| 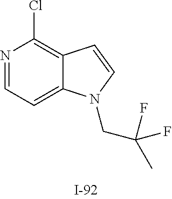 | 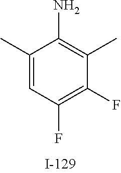 | 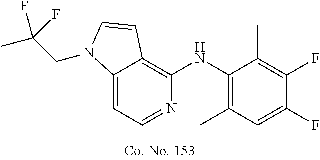 [SMILES: Cc1cc(F)c(F)c(C)c1Nc1nccc2c1ccn2CC(C)(F)F] |
| 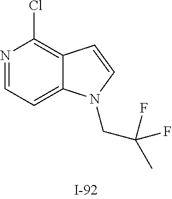 | 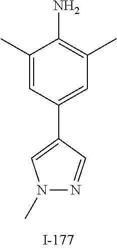 | 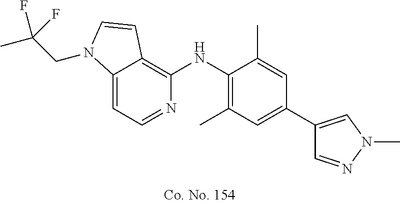 [SMILES: Cc1cc(-c2cnn(C)c2)cc(C)c1Nc1nccc2c1ccn2CC(C)(F)F] |
| 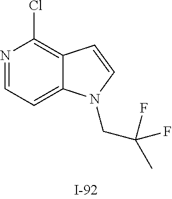 | 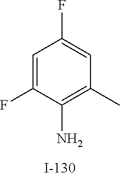 | 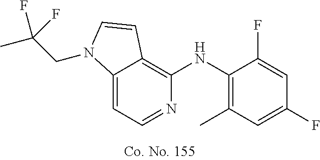 [SMILES: Cc1cc(F)cc(F)c1Nc1nccc2c1ccn2CC(C)(F)F] |
| 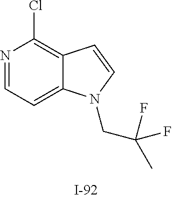 | 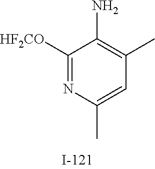 | 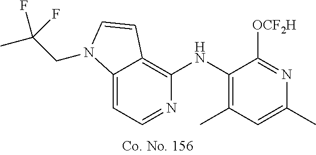 |
| 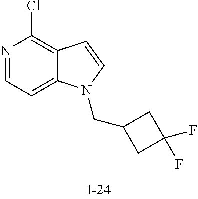 [SMILES: FC1(F)CC(Cn2ccc3c(Cl)nccc32)C1] | 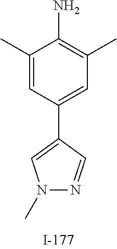 | 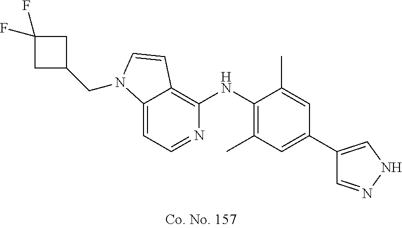 [SMILES: Cc1cc(-c2cn[nH]c2)cc(C)c1Nc1nccc2c1ccn2CC1CC(F)(F)C1] |
| 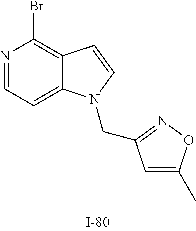 [SMILES: Cc1cc(Cn2ccc3c(Br)nccc32)no1] | 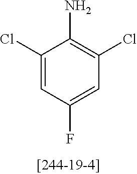 | 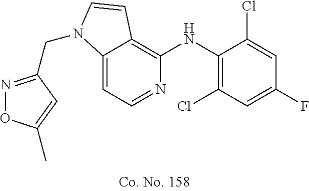 [SMILES: Cc1cc(Cn2ccc3c(Nc4c(Cl)cc(F)cc4Cl)nccc32)no1] |
| 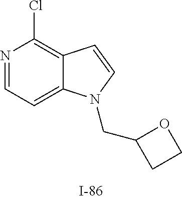 [SMILES: Clc1nccc2c1ccn2CC1CCO1] | 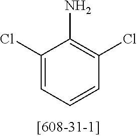 | 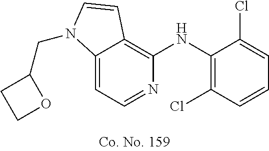 [SMILES: Clc1cccc(Cl)c1Nc1nccc2c1ccn2CC1CCO1] |
| 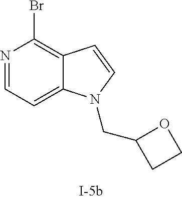 [SMILES: Brc1nccc2c1ccn2CC1CCO1] | 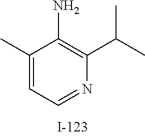 | 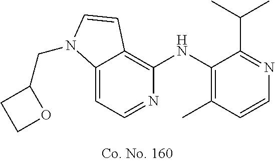 [SMILES: Cc1ccnc(C(C)C)c1Nc1nccc2c1ccn2CC1CCO1] |
| 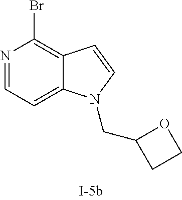 [SMILES: Brc1nccc2c1ccn2CC1CCO1] | 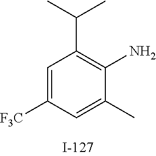 | 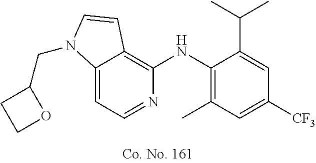 [SMILES: Cc1cc(C(F)(F)F)cc(C(C)C)c1Nc1nccc2c1ccn2CC1CCO1] |
| 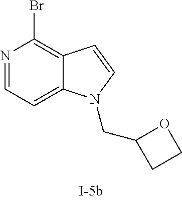 [SMILES: Brc1nccc2c1ccn2CC1CCO1] | 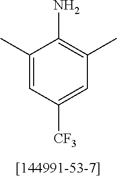 | 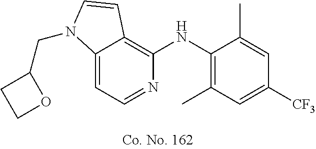 [SMILES: Cc1cc(C(F)(F)F)cc(C)c1Nc1nccc2c1ccn2CC1CCO1] |
| 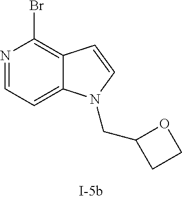 [SMILES: Brc1nccc2c1ccn2CC1CCO1] | 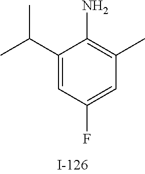 | 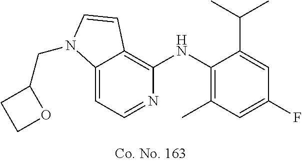 [SMILES: Cc1cc(F)cc(C(C)C)c1Nc1nccc2c1ccn2CC1CCO1] |

### Preparation of Compound 164

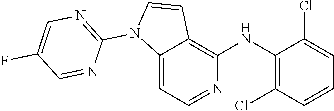 [SMILES: Fc1cnc(-n2ccc3c(Nc4c(Cl)cccc4Cl)nccc32)nc1]

### Preparation of Compound 165

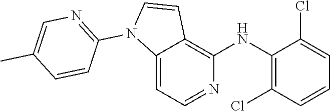 [SMILES: Cc1ccc(-n2ccc3c(Nc4c(Cl)cccc4Cl)nccc32)nc1]

To a mixture of I-134 (50.0 mg, 0.17 mmol), XPhos (8.27 mg, 17.4 μmol), Cs₂CO₃ (0.17 g, 0.52 mmol) and 2,6-dichloroaniline [608-31-1] (30.9 mg, 0.19 mmol) in toluene (20 mL) was added Pd₂dba₃ (15.9 mg, 17.4 μmol) under N2 atmosphere. The reaction mixture was stirred at 90° C. for 12 h. The mixture was extracted with DCM (3×10 mL). The combined organic layers were dried (Na₂SO₄), filtered and evaporated in vacuo. The crude mixture was purified by preparative high-performance liquid chromatography (column: Gemini 150*25 5u, mobile phase: water (0.05% ammonia hydroxide v/v)/CH₃CN, gradient from 25:75 to 45:55) to afford compound 165 (15.1 mg, 23%) as a white solid.

The following compound was prepared in an analogous manner to that described for compound 165 starting from the indicated starting material and reagent.

| STARTING MATERIAL | REAGENT | COMPOUND |
|---|---|---|
| 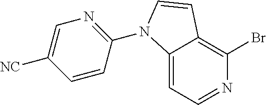 [SMILES: N#Cc1ccc(-n2ccc3c(Br)nccc32)nc1] I-133 | 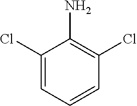 [608-31-1] | 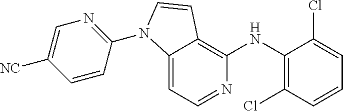 [SMILES: N#Cc1ccc(-n2ccc3c(Nc4c(Cl)cccc4Cl)nccc32)nc1] Co. No. 166 |

### Preparation of Compound 167

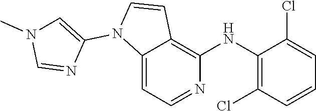 [SMILES: Cn1cnc(-n2ccc3c(Nc4c(Cl)cccc4Cl)nccc32)c1]

Pd₂dba₃ (18.7 mg, 20.4 μmol), XantPhos (29.5 mg, 51.0 μmol) and Cs₂CO₃ (249 mg, 0.77 mmol) were added to a stirred mixture of 6-dichloroaniline [608-31-1] (107 mg, 0.66 mmol) and I-139 (118 mg, 0.51 mmol) in DMF (5.1 mL). The reaction mixture was stirred at 105° C. for 12 h in a sealed tube. The mixture was cooled to room temperature and partitioned between NaHCO₃ (sat., aq.) and EtOAc. The aqueous phase was extracted with EtOAc (twice). The combined organic phases were washed with brine, dried (MgSO₄), filtered and concentrated in vacuo. The crude mixture was purified by flash column chromatography (silica, heptane/EtOAc, gradient from 100:0 to 0:100) to afford compound 167 (117 mg, 64%) as a white solid.

The following compounds were prepared in an analogous manner to that described for compound 167 using the indicated starting material and reagents.

| STARTING MATERIAL | REAGENT | COMPOUND |
|---|---|---|
| 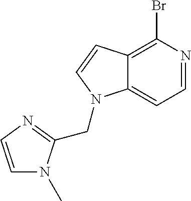 [SMILES: Cn1ccnc1Cn1ccc2c(Br)nccc21] I-87 | 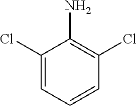 [608-31-1] | 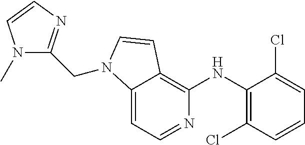 [SMILES: Cn1ccnc1Cn1ccc2c(Nc3c(Cl)cccc3Cl)nccc21] Co. No. 168 |
| 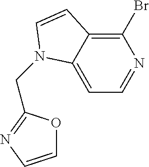 [SMILES: Brc1nccc2c1ccn2Cc1ncco1] I-89 | 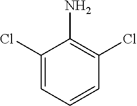 [608-31-1] | 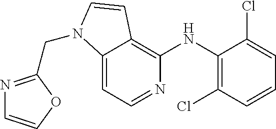 [SMILES: Clc1cccc(Cl)c1Nc1nccc2c1ccn2Cc1ncco1] Co. No. 169 |
| 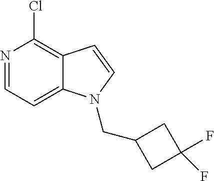 [SMILES: FC1(F)CC(Cn2ccc3c(Cl)nccc32)C1] I-24 | 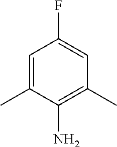 [392-70-1] | 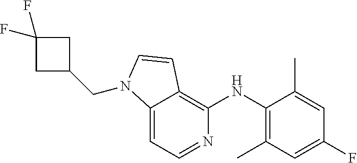 [SMILES: Cc1cc(F)cc(C)c1Nc1nccc2c1ccn2CC1CC(F)(F)C1] Co. No. 170 |
| 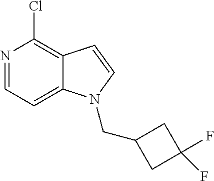 [SMILES: FC1(F)CC(Cn2ccc3c(Cl)nccc32)C1] I-24 | 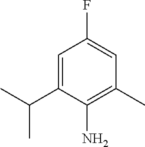 I-126 | 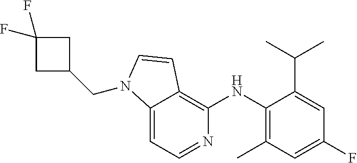 [SMILES: Cc1cc(F)cc(C(C)C)c1Nc1nccc2c1ccn2CC1CC(F)(F)C1] Co. No. 171 |
| 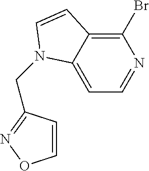 [SMILES: Brc1nccc2c1ccn2Cc1ccon1] I-88 | 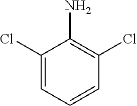 [608-31-1] | 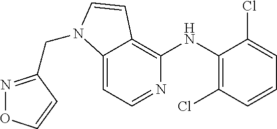 [SMILES: Clc1cccc(Cl)c1Nc1nccc2c1ccn2Cc1ccon1] Co. No. 172 |
| 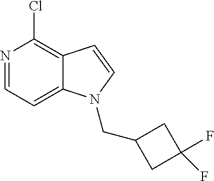 [SMILES: FC1(F)CC(Cn2ccc3c(Cl)nccc32)C1] I-24 | 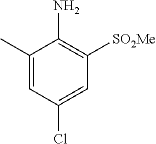 I-146 | 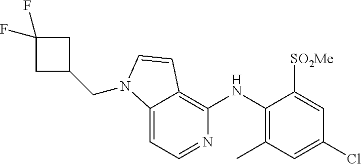 [SMILES: Cc1cc(Cl)cc(S(C)(=O)=O)c1Nc1nccc2c1ccn2CC1CC(F)(F)C1] Co. No. 173 |
| 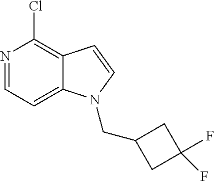 [SMILES: FC1(F)CC(Cn2ccc3c(Cl)nccc32)C1] I-24 | 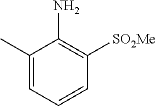 I-148 | 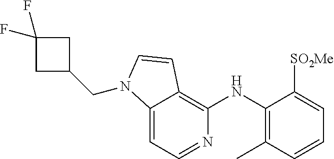 [SMILES: Cc1cccc(S(C)(=O)=O)c1Nc1nccc2c1ccn2CC1CC(F)(F)C1] Co. No. 174 |
| 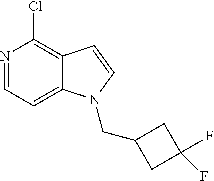 [SMILES: FC1(F)CC(Cn2ccc3c(Cl)nccc32)C1] I-24 | 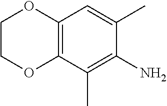 I-179 | 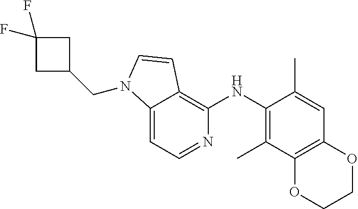 [SMILES: Cc1cc2c(c(C)c1Nc1nccc3c1ccn3CC1CC(F)(F)C1)OCCO2] Co. No. 175 |
| 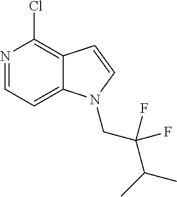 [SMILES: CC(C)C(F)(F)Cn1ccc2c(Cl)nccc21] I-150 | 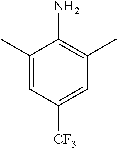 [144991-53-7] | 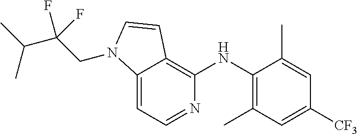 [SMILES: Cc1cc(C(F)(F)F)cc(C)c1Nc1nccc2c1ccn2CC(F)(F)C(C)C] Co. No. 176 |
| 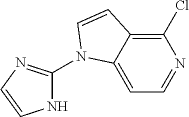 [SMILES: Clc1nccc2c1ccn2-c1ncc[nH]1] I-155 | 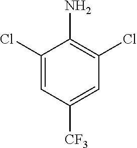 [SMILES: Nc1c(Cl)cc(C(F)(F)F)cc1Cl] [344-19-4] | 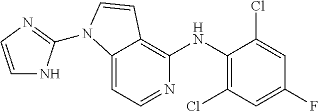 [SMILES: Fc1cc(Cl)c(Nc2nccc3c2ccn3-c2ncc[nH]2)c(Cl)c1] Co. No. 177 |
| Mixture of I-150 and I-184 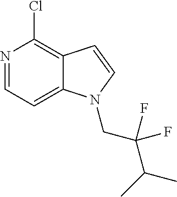 [SMILES: CC(C)C(F)(F)Cn1ccc2c(Cl)nccc21] I-150 | 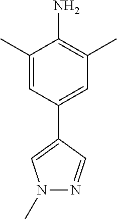 I-177 | 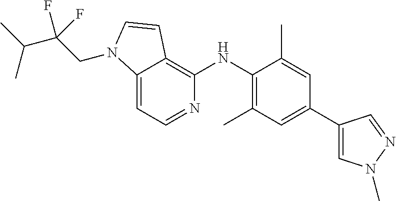 [SMILES: Cc1cc(-c2cnn(C)c2)cc(C)c1Nc1nccc2c1ccn2CC(F)(F)C(C)C] Co. No. 178 |
| 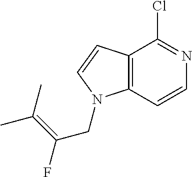 [SMILES: CC(C)=C(F)Cn1ccc2c(Cl)nccc21] I-184 | | 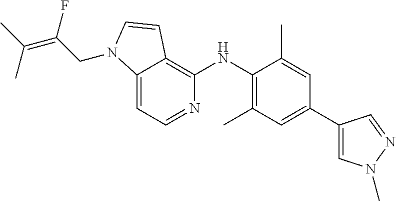 [SMILES: CC(C)=C(F)Cn1ccc2c(Nc3c(C)cc(-c4cnn(C)c4)cc3C)nccc21] Co. No. 208 |
| 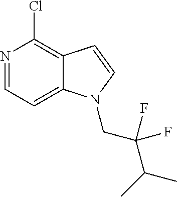 [SMILES: CC(C)C(F)(F)Cn1ccc2c(Cl)nccc21] I-150 | 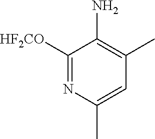 I-155 | 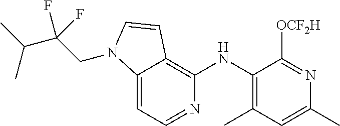 Co. No. 179 |
| 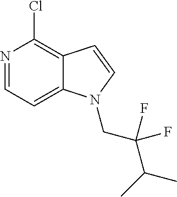 [SMILES: CC(C)C(F)(F)Cn1ccc2c(Cl)nccc21] I-150 | 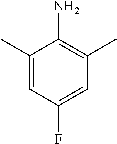 [392-70-1] | 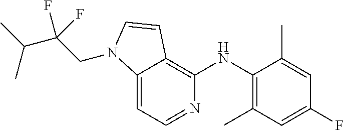 [SMILES: Cc1cc(F)cc(C)c1Nc1nccc2c1ccn2CC(F)(F)C(C)C] Co. No. 180 |
| 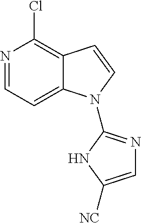 [SMILES: N#Cc1cnc(-n2ccc3c(Cl)nccc32)[nH]1] I-157 | 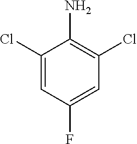 [344-19-4] | 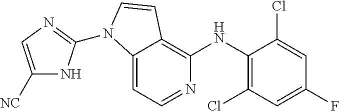 [SMILES: N#Cc1cnc(-n2ccc3c(Nc4c(Cl)cc(F)cc4Cl)nccc32)[nH]1] Co. No. 181 |
| 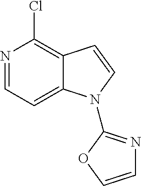 [SMILES: Clc1nccc2c1ccn2-c1ncco1] I-156 | 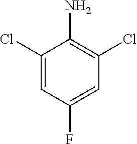 [344-19-4] | 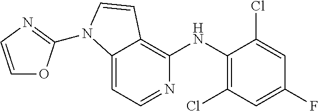 [SMILES: Fc1cc(Cl)c(Nc2nccc3c2ccn3-c2ncco2)c(Cl)c1] Co. No. 182 |
| 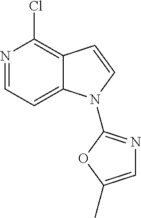 [SMILES: Cc1cnc(-n2ccc3c(Cl)nccc32)o1] I-162 | 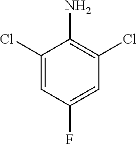 [344-19-4] | 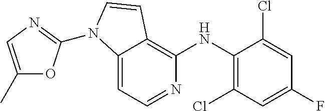 [SMILES: Cc1cnc(-n2ccc3c(Nc4c(Cl)cc(F)cc4Cl)nccc32)o1] Co. No. 183 |
| 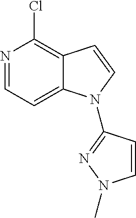 [SMILES: Cn1ccc(-n2ccc3c(Cl)nccc32)n1] I-158 | 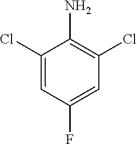 [344-19-4] | 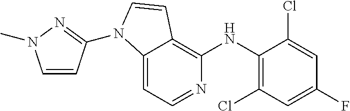 [SMILES: Cn1ccc(-n2ccc3c(Nc4c(Cl)cc(F)cc4Cl)nccc32)n1] Co. No. 184 |
| 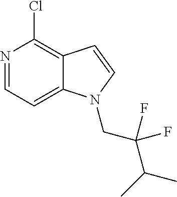 [SMILES: CC(C)C(F)(F)Cn1ccc2c(Cl)nccc21] I-150 | 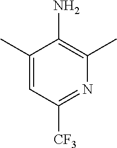 I-164 | 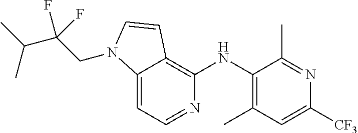 [SMILES: Cc1cc(C(F)(F)F)nc(C)c1Nc1nccc2c1ccn2CC(F)(F)C(C)C] Co. No. 185 |
| 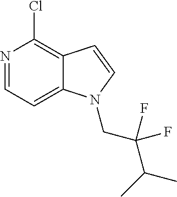 [SMILES: CC(C)C(F)(F)Cn1ccc2c(Cl)nccc21] I-150 | 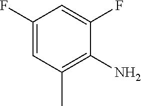 [1464825-76-0] | 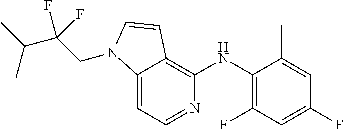 [SMILES: Cc1cc(F)cc(F)c1Nc1nccc2c1ccn2CC(F)(F)C(C)C] Co. No. 186 |
| 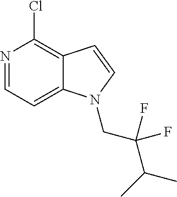 [SMILES: CC(C)C(F)(F)Cn1ccc2c(Cl)nccc21] I-150 | 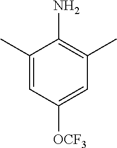 I-147 | 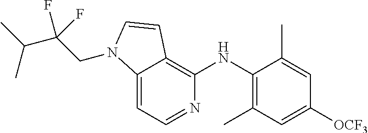 [SMILES: Cc1cc(OC(F)(F)F)cc(C)c1Nc1nccc2c1ccn2CC(F)(F)C(C)C] HCl salt Co. No. 187 |
| 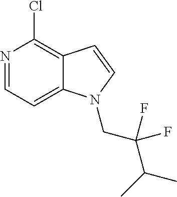 [SMILES: CC(C)C(F)(F)Cn1ccc2c(Cl)nccc21] I-150 | 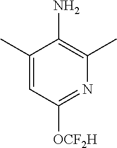 I-154 | 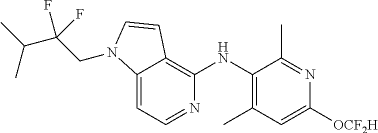 HCl salt Co. No. 188 |
| 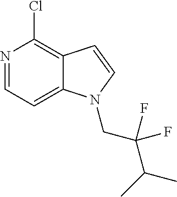 [SMILES: CC(C)C(F)(F)Cn1ccc2c(Cl)nccc21] I-150 | 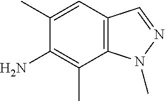 I-182 | 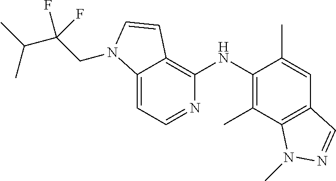 [SMILES: Cc1cc2cnn(C)c2c(C)c1Nc1nccc2c1ccn2CC(F)(F)C(C)C] HCl salt Co. No. 189 |

### Preparation of Compound 190

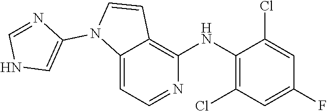 [SMILES: Fc1cc(Cl)c(Nc2nccc3c2ccn3-c2c[nH]cn2)c(Cl)c1]

HCl (4M in 1,4-dioxane, 4.9 mL, 19.6 mmol) was added to a stirred solution of I-171 (200 mg, 0.33 mmol) in MeOH (3.2 mL). The reaction mixture was stirred at 55° C. for 2 h and the solvent was evaporated in vacuo. The crude mixture was purified by reverse phase chromatography (25 mM NH₄HCO₃/(CH₃CN/MeOH 1:1), gradient from 81:19 to 45:55). The product was triturated in Et₂O to afford compound 190 (67 mg, 55%) as a white solid.

The following compound was obtained in an analogous manner to that described for compound 190 from the indicated starting material and reagent.

| STARTING MATERIAL | COMPOUND |
|---|---|
| 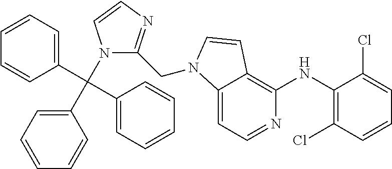 [SMILES: Clc1cccc(Cl)c1Nc1nccc2c1ccn2Cc1nccn1C(c1ccccc1)(c1ccccc1)c1ccccc1] I-183 | 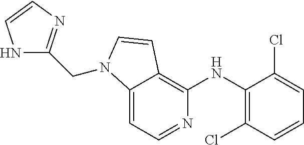 [SMILES: Clc1cccc(Cl)c1Nc1nccc2c1ccn2Cc1ncc[nH]1] Co. No. 191 |

### Preparation of Compound 192

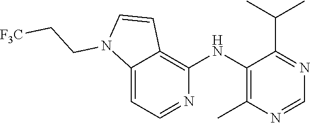 [SMILES: Cc1ncnc(C(C)C)c1Nc1nccc2c1ccn2CCC(F)(F)F]

4-Methyl-6-propan-2-ylpyrimidin-5-amine [1368911-16-3] (59.0 mg, 0.39 mmol) and I-143 (97.0 mg, 0.39 mmol) were added to a stirred solution of Pd(OAc)₂ (3.50 mg, 15.6 μmol), XantPhos (18.1 mg, 31.2 μmol) and Cs₂CO₃ (381 mg, 1.17 mmol) in 1,4-dioxane (10 mL) while N2 was bubbling. The reaction mixture was stirred at 105° C. for 18 h. The mixture was diluted with EtOAc and water. The organic layer was washed with water (twice) and brine, dried (MgSO₄), filtered and the solvents were evaporated in vacuo. The crude mixture was purified by reverse phase chromatography (25 mM NH₄HCO₃/(CH₃CN/MeOH 1:1), gradient from 72:28 to 36:64). The product was triturated in DIPE to afford compound 192 (20 mg, 14%) as a pale white solid.

The following compound was obtained in an analogous manner to that described for compound 192 from the indicated intermediate and reagent.

| INTERMEDIATE | REAGENT | COMPOUND |
|---|---|---|
| 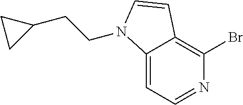 [SMILES: Brc1nccc2c1ccn2CCC1CC1] I-90 | 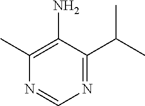 [1368911-16-3] | 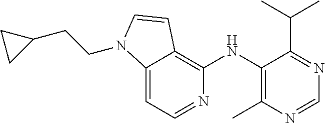 [SMILES: Cc1ncnc(C(C)C)c1Nc1nccc2c1ccn2CCC1CC1] Co. No. 193 |

### Preparation of Compound 194

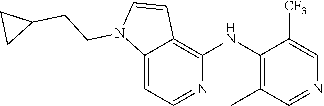 [SMILES: Cc1cncc(C(F)(F)F)c1Nc1nccc2c1ccn2CCC1CC1]

Pd₂dba₃ (20.5 mg, 22.4 μmol), Xantphos (25.9 mg, 44.7 μmol) and Cs₂CO₃ (219 mg, 0.67 mmol) were added to a solution of 4-bromo-3-methyl-5-(trifluoromethyl)pyridine [1211583-82-2] (107 mg, 0.45 mmol) in 1,4-dioxane (15 mL) while N2 was bubbling. After 10 min, I-90 (90.0 mg, 0.45 mmol) was added. The reaction mixture was stirred at room temperature for 10 min, and at 90° C. for 12 h in a sealed tube. The mixture was diluted with water and extracted with EtOAc (3 times). The combined organic layers were dried (MgSO₄), filtered and evaporated in vacuo. The crude mixture was purified by reverse phase (25 mM NH₄HCO₃/(CH₃CN/MeOH 1:1), gradient from 59:41 to 17:83). The product was triturated in DIPE to afford compound 194 (15 mg, 9%) a white solid.

The following compound was obtained in an analogous manner to that described for compound 194 form the indicated starting material and aniline.

| STARTING MATERIAL | ANILINE | COMPOUND |
|---|---|---|
| 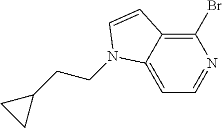 [SMILES: Brc1nccc2c1ccn2CCC1CC1] I-90 | 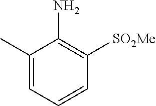 I-148 | 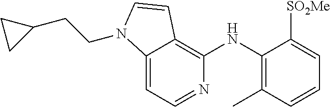 [SMILES: Cc1cccc(S(C)(=O)=O)c1Nc1nccc2c1ccn2CCC1CC1] Co. No. 195 |
| 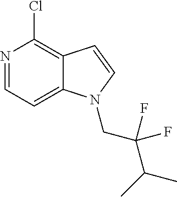 [SMILES: CC(C)C(F)(F)Cn1ccc2c(Cl)nccc21] I-150 | 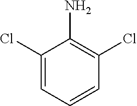 [608-31-1] | 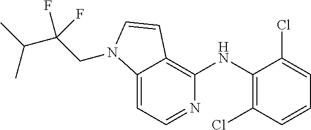 [SMILES: CC(C)C(F)(F)Cn1ccc2c(Nc3c(Cl)cccc3Cl)nccc21] Co. No. 196 |
| 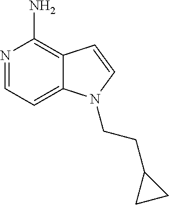 [SMILES: Nc1nccc2c1ccn2CCC1CC1] I-96 | 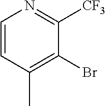 [1448776-80-4] | 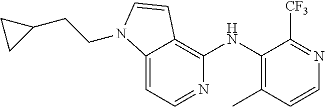 [SMILES: Cc1ccnc(C(F)(F)F)c1Nc1nccc2c1ccn2CCC1CC1] HCl salt Co. No. 197 |

### Preparation of Compound 198

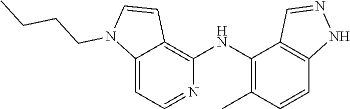 [SMILES: CCCCn1ccc2c(Nc3c(C)ccc4[nH]ncc34)nccc21]

I-176 (120 mg, 0.27 mmol) was dissolved in TFA (1.99 mL, 26.8 mmol). The reaction mixture was stirred at 95° C. for 12 h and the solvent was evaporated in vacuo. The mixture was diluted with NaHCO₃ and extracted with DCM. The organic layer was dried (MgSO₄), filtered and concentrated under reduced pressure. The crude mixture was purified by flash column chromatography (silica, heptane/EtOAc, gradient from 100:0 to 0:100). A second purification was performed by reverse phase (25 mM NH₄HCO₃/(CH₃CN/MeOH 1:1), gradient from 70:30 to 27:73). The product was triturated in Et₂O to afford compound 198 (11.2 mg, 13%) as a beige solid.

### Preparation of Compound 199

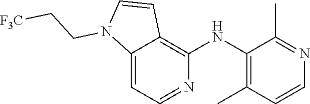 [SMILES: Cc1ccnc(C)c1Nc1nccc2c1ccn2CCC(F)(F)F]

Pd₂dba₃ (24.8 mg, 27 μmol), Xantphos (26.1 mg, 45 μmol) and K₃PO₄ (275 mg, 1.30 mmol) were added to a solution of I-143 (112 mg, 0.45 mmol) in 1,4-dioxane (10 mL) while N₂ was bubbling. After 10 min, 3-amino-2,4-dimethylpyridine [1073-21-8] (55.0 mg, 0.45 mmol) was added. the reaction mixture was stirred at room temperature for 10 min in a sealed tube and at 90° C. for 16 h. The mixture was diluted with water and extracted with EtOAc. The organic layer was dried (MgSO₄), filtered and the solvents were evaporated in vacuo. The crude mixture was purified by flash column chromatography (silica, heptane/EtOAc, gradient from 100:0 to 75:25). The product was dissolved in DCM (3 mL) and HCl (4M) was added (1.0 eq). The mixture was concentrated in vacuo and the product was cristallizated from Et₂O. The residue was purified by reverse phase (25 mM NH₄HCO₃/(CH₃CN/MeOH, 1:1), gradient from 81:19 to 45:55). The product was triturated in Et₂O to afford compound 199 (22.5 mg, 15%) as a white foam.

The following compound was obtained in an analogous manner to that described for compound 199 from the indicated starting material and reagent.

| STARTING MATERIAL | REAGENT | COMPOUND |
|---|---|---|
| 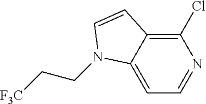 [SMILES: FC(F)(F)CCn1ccc2c(Cl)nccc21] I-143 | 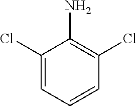 [608-31-1] | 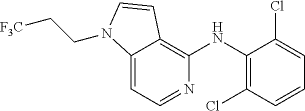 [SMILES: FC(F)(F)CCn1ccc2c(Nc3c(Cl)cccc3Cl)nccc21] Co. No. 200 |

### Preparation of Compound 201

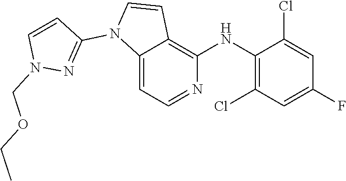 [SMILES: CCOCn1ccc(-n2ccc3c(Nc4c(Cl)cc(F)cc4Cl)nccc32)n1]

HCl (12M solution, 0.82 mL, 9.9 mmol) was added to mixture of I-192 and I-193 (325 mg, 0.66 mmol) in EtOH (5 mL) at room temperature. The reaction mixture was stirred at 70° C. for 8 h. Additional amount of HCl (12M solution, 0.50 mL, 6.0 mmol) was added and the reaction mixture was stirred at 70° C. for another 8 h. The mixture was cooled to room temperature and the solvents were concentrated in vacuo. The crude mixture was dissolved in EtOAc (30 mL) and washed with NaHCO₃ (sat., aq. 10×5 mL). The combined organic layers were dried (MgSO₄), filtered and concentrated in vacuo. The crude mixture was purified by flash column chromatography (silica, heptane/EtOAc, gradient from 100:0 to 65:35). The product was triturated in DIPE to afford compound 201 (13.2 mg, 4%, 95% purity).

### Preparation of Compounds 202 and 203

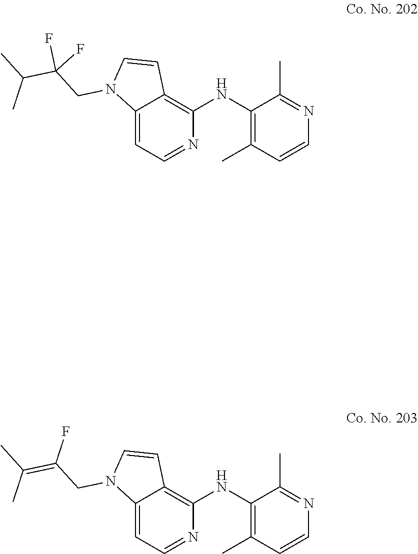 [SMILES: Cc1ccnc(C)c1Nc1nccc2c1ccn2CC(F)(F)C(C)C ; CC(C)=C(F)Cn1ccc2c(Nc3c(C)ccnc3C)nccc21]

Pd₂dba₃ (42.0 mg, 45.9 μmol), Xantphos (44.3 mg, 76.5 μmol) and K₃PO₄ (468 mg, 2.20 mmol) were added to a mixture of I-186 (containing 50% of I-187, 232 mg, 0.77 mmol) in THF (10 mL) while N₂ was bubbling. After 10 min, 3-amino-2,4-dimethylpyridine [1073-21-8] (93.5 mg, 0.77 mmol) was added. The reaction mixture was stirred at room temperature for 10 min, and at 90° C. for 16 h in a sealed tube. The mixture was diluted with water and extracted with EtOAc. The combined organic layers were dried (MgSO₄), filtered and the solvents were evaporated in vacuo. The crude mixture was purified by flash column chromatography (silica, heptane/EtOAc, gradient from 100:0 to 70:30). A second purification was performed by reverse phase (HCOOH (0.1%)/(CH₃CN/MeOH (1:1)), gradient from 95:5 to 63:37) to afford compound 202 and compound 203. The residues were separately taken up in DCM and treated with HCl 4N in 1,4-dioxane (1 eq.). The solvents were evaporated in vacuo. The products were finally tritured in Et₂O to afford compound 202 (29.7 mg, 10%) as a HCl salt and compound 203 (31.6 mg, 11%) as a HCl salt.

### Preparation of Compound 101

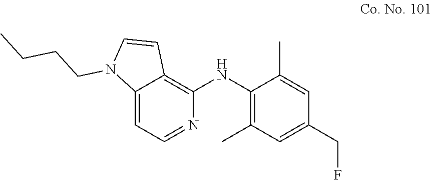 [SMILES: CCCCn1ccc2c(Nc3c(C)cc(CF)cc3C)nccc21]

HCl (4M in dioxane, 0.352 mL, 1.41 mmol) was added to a stirred solution of I-74 (60 mg, 0.141 mmol) in 1,4-dioxane (1.2 mL) and the mixture was stirred at rt for 2 h. Then additional HCl (106 μL) was added and the rm was stirred at rt for 60 h. Then further HCl (106 μL) was added and the rm was stirred at rt for 48 h. The rm was concentrated and purified by column chromatography (silica gel; eluent: DCM/7N NH₃ in MeOH 100/0 to 98/2) to afford 38 mg of Co. No. 101, which was further purified via Prep SFC (stationary phase: Chiralpak Daicel IC 20×250 mm; mobile phase: CO₂, EtOH+0.4 iPrNH₂), to yield a white solid that was dried in a vacuum oven at 55° C. to yield Co. No. 101 (17 mg, 37%).

### Preparation of Compound 102

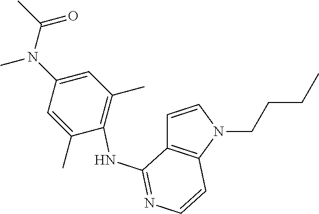 [SMILES: CCCCn1ccc2c(Nc3c(C)cc(N(C)C(C)=O)cc3C)nccc21]

To a solution of Co. No. 62 (152.4 mg, 0.435 mmol) in DMF (1.5 mL) was added portionwise NaH (60% dispersion in mineral oil, 20.2 mg, 0.505 mmol) under nitrogen at 0° C. The reaction mixture was allowed to reach rt and stirred 30 min. Dimethyl sulfate (42 μL, 1.333 g/mL, 0.444 mmol) was added dropwise at 0° C. and the mixture was stirred for 3 h. NaHCO₃ sat. sol. was added and the OL was extracted with EtOAc, then washed with water and brine, then dried over MgSO₄ and the solvent was removed. To help removing DMF, the residue was diluted twice in MIK and co-evaporated under vacuum. This fraction was then purified via Prep HPLC (Stationary phase: RP XBridge Prep C18 OBD-10 μm, 30×150 mm; mobile phase: 0.25% NH₄HCO₃ solution in water, CH₃CN) to yield Co. No. 102 (23 mg, yield 14.51%) as a pale brownish powder.

### Preparation of Compound 204

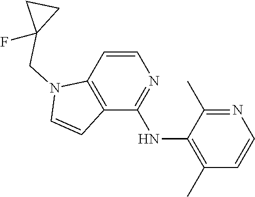 [SMILES: Cc1ccnc(C)c1Nc1nccc2c1ccn2CC1(F)CC1]

I-50 (36.8 mg, 0.15 mmol) was dissolved in DMF (1.2 mL). NaH (60% dispersion in mineral oil, 6.79 mg, 0.17 mmol) was added at 0° C. and the mixture was stirred at room temperature. When gas evolution stopped, (1-fluorocyclopropyl)methyl methanesulphonate (93.3 mg, 0.56 mmol) was added at 0° C. The reaction mixture was stirred at room temperature. The reaction was quenched with water and diluted with EtOAc. The aqueous layer was extracted with EtOAc (3 times). The combined organic layers were washed with brine, dried (MgSO₄), filtered and concentrated in vacuo. The crude mixture was purified by Prep HPLC (stationary phase: XBridge Prep C18 3.5 μm, 4.6×100 mm, mobile phase: 0.2% NH₄HCO₃ (0.2% solution in water)/CH₃CN) to afford compound 204 (11 mg, 23%).

### Preparation of Compound 205

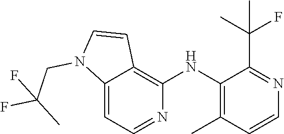 [SMILES: Cc1ccnc(C(C)(C)F)c1Nc1nccc2c1ccn2CC(C)(F)F]

To a mixture of I-168 (269 mg, 0.37 mmol, 50% purity) in DCM (2 mL) was added DAST [38078-09-0] (0.1 mL, 0.76 mmol) at 0° C. The reaction mixture was stirred at 0° C. for 1 h, diluted with NaHCO₃ and extracted with DCM. The combined organic extracts were washed with water, dried (MgSO₄), filtered and concentrated in vacuo. The crude mixture was purified via Prep SFC (stationary phase: Chiralpak Diacel AD 20×250 mm, mobile phase: CO₂, i-PrOH+0.4% i-PrNH₂) to afford compound 205 (19 mg, 14%).

### Preparation of Compound 206

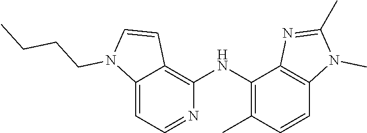 [SMILES: CCCCn1ccc2c(Nc3c(C)ccc4c3nc(C)n4C)nccc21]

Compound 11 (71.1 mg, 0.21 mmol) was dissolved in DMF (1 mL) under N₂ atmosphere. NaH (60% dispersion in mineral oil, 11.1 mg, 0.28 mmol) was added and the mixture was stirred at room temperature for 30 min. Mel (36.3 mg, 0.26 mmol) was added dropwise and the reaction mixture was stirred at room temperature for 1 h. The reaction was quenched with water. The organic layer was extracted with DCM, dried (MgSO₄), filtered and evaporated in vacuo. The crude mixture was purified by reverse phase. The residue was purified via prep SFC (stationary phase: Chiralpak Diacel AD 20×250 mm, mobile phase: C02, EtOH+0.4% i-PrNH₂) to afford compound 206 (21.3 mg, 29%) as a white foam.

### Preparation of Compound 209

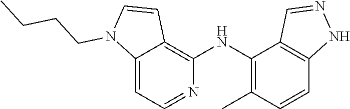 [SMILES: CCCCn1ccc2c(Nc3c(C)ccc4[nH]ncc34)nccc21]

I-176 (120 mg, 0.27 mmol) was dissolved in TFA (1.98 mL). The reaction mixture was stirred at 95° C. for 13 h, cooled down and the solvent was evaporated in vacuo. The mixture was diluted with NaHCO₃ and extracted with DCM. The combined organic layers were dried (MgSO₄), filtered and concentrated in vacuo. The crude mixture was purified by flash column chromatography (silica; EtOAc in heptane, gradient from 0/100 to 100/0). The desired fractions were collected and concentrated in vacuo. A second purification was performed by purified by reverse phase ([25 mM NH₄HCO₃]/[MeCN:MeOH (1:1)], gradient from 70:30 to 27:73). The desired fractions were collected and concentrated in vacuo. The product was triturated in Et₂O to afford compound 209 (11.2 mg, 13%) as a beig solid.

### Analytical Part

### Melting Points

Values are either peak values or melt ranges, and are obtained with experimental uncertainties that are commonly associated with this analytical method. DSC823e or DSC1 STAR (indicated as (a)) & Mettler Toledo MP50:

For a number of compounds, melting points were determined with a DSC823e or a DSC1 STAR (Mettler-Toledo). Melting points were measured with a temperature gradient of 10° C./minute. Maximum temperature was 300° C.

For a number of compounds, melting points were determined with a MP50 (Mettler-Toledo) (indicated as (b)). Melting points were measured with a temperature gradient of 10° C./minute.

### LCMS

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻ etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, HSS” High Strength Silica, “CSH” charged surface hybrid, “UPLC” Ultra Performance Liquid Chromatography, “DAD” Diode Array Detector.

TABLE 1
LC-MS Methods (Flow expressed in mL/min; column temperature (T) in
° C.; Run time in min).

| | | | | | Flow | Run |
|---|---|---|---|---|---|---|
| Method | | | | | [symbol] | time |
| code | Instrument | Column | Mobile phase | Gradient | Col T | (min) |
| 1 | Waters: | Waters : | A: 10 mM | From 100% A | 0.6 | 3.5 |
| | Acquity ® | BEH | CH₃COONH₄ | to 5% A in | [symbol] | |
| | UPLC ® -DAD | (1.8 μm, | in 95% H₂O + | 2.10 min, to 0% | 55 | |
| | and SQD | 2.1*100 mm) | 5% CH₃CN | A in 0.90 min | | |
| | | | B: CH₃CN | to 5% A in | | |
| | | | | 0.5 min | | |
| 2 | Waters: | Waters: | A: 10 mM | From 95% A to | 0.8 | 2 |
| | Acquity ® | BEH C18 | CH₃COONH₄ | 5% A in | [symbol] | |
| | UPLC ® -DAD | (1.7 μm, | in 95% H₂O + | 1.3 min, | 55 | |
| | and SQD | 2.1*50 mm) | 5% CH₃CN | held for 0.7 min | | |
| | | | B: CH₃CN | | | |
| 3 | Waters: | Waters: | A: 10 mM | From 100% A | 0.7 | 3.5 |
| | Acquity ® | HSS T3 | CH3COONH4 | to | [symbol] | |
| | UPLC ® -DAD | (1.8 μm, | in 95% H2O + | 5% A in | 55 | |
| | and SQD | 2.1*100 mm) | 5% CH₃CN | 2.10 min, | | |
| | | | B: CH₃CN | to 0% A in | | |
| | | | | 0.90 min, | | |
| | | | | to 5% A in | | |
| | | | | 0.5 min | | |
| 4 | Waters: | Waters: | A: 10mM | From 100% A | 0.6 | 3.5 |
| | Acquity ® | HSS T3 | CH₃COONH₄ | to 5% A in | [symbol] | |
| | UPLC ® -DAD, | (1.8 μm, | in 95% H₂O + | 2.10 min to 0% | 55 | |
| | SQD and | 2.1*100 mm) | 5% CH₃CN | A in 0.90 min, | | |
| | ELSD | | B: CH₃CN | to 5% A in | | |
| | | | | 0.5min | | |
| 5 | Waters: | Waters : | A: 0.1% | From 100% A | 0.6 | 3.5 |
| | Acquity ® | BEH | NH₄HCO₃ in | to 5% A in | [symbol] | |
| | UPLC ® -DAD, | (1.8 μm, | H₂O | 2.10 min, to 0% | 55 | |
| | SQD | 2.1*100 mm) | B: MeOH | A in 0.90 min, | | |
| | | | | to 5% A in | | |
| | | | | 0.5 min | | |
| 6 | Agilent: 1100- | YMC: Pack | A: HCOOH | 95% A to 5% A | 2.6 | 6 |
| | DAD and MSD | ODS-AQ | 0.1% in water, | in 4.8 min, held | | |
| | | (3 μm, | B: CH₃CN | for 1 min, back | | |
| | | 4.6 × 50 mm) | | to 95% A in | | |
| | | | | 0.2 min. | | |
| 7 | Waters: | Waters: | A: 10 mM | From 100% A | 0.6 | 3.5 |
| | Acquity ® | HSS T3 | CH3COONH4 | to | [symbol] | |
| | UPLC ® -DAD | (1.8 μm, | in 95% H2O + | 5% A in | 55 | |
| | and SQD | 2.1*100 mm) | 5% CH3CN | 2.10 min, | | |
| | | | B: CH3CN | to 0% A in | | |
| | | | | 0.90 min, | | |
| | | | | to 5% A in | | |
| | | | | 0.5 min | | |

TABLE 2
Analytical data-melting point (Mp) and LCMS: [M + H]⁺ means the
protonated mass of the free base of the compound, [M − H]⁻ means the deprotonated mass
of the free base of the compound or the type of adduct specified [M + CH3C00]⁻). Rₜ
means retention time (in min). For some compounds, exact mass was determined.

| Co. No. | Mp (° C.) | Rt | UV Area % | [M + H]+ | [M − H]− | LCMS Method |
|---|---|---|---|---|---|---|
| 1 | | 1.52 | 100.00 | 329 | 327 | 1 |
| 2 | | 1.46 | 100.00 | 329 | 327 | 1 |
| 3 | | 0.75 | 100.00 | 400 | 398 | 2 |
| 4 | 161.61^{(a)} | | | | | |
| 5 | 88.69^{(a)} | 0.99 | 100.00 | 315 | 313 | 2 |
| 6 | | 0.84 | 98.74 | 295 | 293 | 2 |
| 7 | | 1.00 | 100.00 | 352 | 350 | 2 |
| 8 | 129.11^{(a)} | 2.11 | 99.05 | 342 | 340 | 3 |
| 8 | 129.11^{(a)} | 0.94 | 100.00 | 359 | 357 | 2 |
| 9 | 146.3^{(b)} | 2.66 | 99.00 | 348 | | 6 |
| 10 | 159.6^{(b)} | 2.27 | 99.00 | 334 | | 6 |
| 11 | | 0.79 | 100.00 | 334 | 332 | 2 |
| 12 | | 1.67 | 81.73 | 321 | 319 | 1 |
| 13 | | 0.83 | 100.00 | 407 | 465 | 2 |
| | | | | | [MCH₃C00]− | |
| 14 | 162.74^{(a)} | 1.04 | 95.17 | 353 | | 2 |
| 15 | | 0.96 | 100.00 | 331 | | 2 |
| 16 | 186.4^{(b)} | 1.71 | 99.00 | 309 | | 6 |
| 17 | | 0.82 | 98.13 | 296 | 294 | 2 |
| 19 | | 1.82 | 100.00 | 324 | 322 | 4 |
| 20 | | 1.63 | 97.76 | 339 | 337 | 1 |
| 21 | | 0.77 | 100.00 | 323 | | 2 |
| 22 | | 1.94 | 100.00 | 323 | 321 | 5 |
| 23 | | 0.98 | 100.00 | 343 | 341 | 2 |
| 24 | | 1.87 | 100.00 | 382 | 380 | 3 |
| 25 | | 1.37 | 100.00 | 309 | 307 | 1 |
| 25a | | 1.29 | 100.00 | 309 | 307 | 1 |
| 25b | | 1.29 | 100.00 | 309 | 307 | 1 |
| 26 | 186.02^{(a)} | 1.42 | 100.00 | 334 | 332 | 1 |
| 27 | | 1.52 | 98.57 | 331 | 329 | 1 |
| 28 | 129.24^{(a)} | 0.78 | 100.00 | 337 | 335 | 2 |
| 29 | | 1.18 | 100.00 | 309 | 307 | 1 |
| 30 | 128.42^{(a)} | 0.67 | 100.00 | 329 | 327 | 2 |
| 31 | | 1.00 | 96.76 | 313 | | 2 |
| 32 | | 1.33 | 100.00 | 337 | 335 | 1 |
| 33 | 172.06^{(a)} | 0.95 | 100.00 | 309 | 307 | 2 |
| 34 | | 1.96 | 100.00 | 313 | 311 | 4 |
| 35 | 161.13^{(a)} | | | | | |
| 36 | | 0.75 | 100.00 | 346 | 344 | 2 |
| 37 | 112.9^{(b)} | 2.26 | 99.00 | 284 | | 6 |
| 38 | 83.99^{(a)} | 1.24 | 96.35 | 323 | 321 | 4 |
| 39 | | 2.07 | 100.00 | 348 | | 1 |
| 40 | 133.12^{(a)} | 0.89 | 100.00 | 336 | 334 | 2 |
| 41 | | 1.59 | 97.97 | 293 | 291 | 1 |
| 41 | 176.68^{(a)} | 1.62 | 100.00 | 293 | 291 | 1 |
| 42 | 130.96^{(a)} | 1.98 | 100.00 | 335 | 333 | 4 |
| 43 | | 1.96 | 100.00 | 325 | 323 | 4 |
| 44 | | 0.98 | 96.91 | 313 | | 2 |
| 45 | 168.1^{(b)} | 2.16 | 99.00 | 363 | | 6 |
| 46 | 121.53^{(b)} | 1.03 | 96.25 | 327 | 325 | 2 |
| 47 | | 0.92 | 100.00 | 309 | 307 | 2 |
| 49 | | 0.90 | 100.00 | 307 | 305 | 2 |
| 50 | 140.92^{(a)} | 0.86 | 96.42 | 345 | 343 | 2 |
| 51 | | 1.00 | 96.64 | 358 | | 2 |
| 52 | | 0.89 | 98.39 | 295 | 293 | 2 |
| 53 | 206.5^{(b)} | 2.55 | 98.00 | 352 | | 6 |
| 54 | 213.3^{(b)} | 2.66 | 99.00 | 374 | | 6 |
| 55 | 203.12^{(a)} | 0.93 | 100.00 | 391 | 449 | 2 |
| | | | | | [MCH₃C00]⁻ | |
| 56 | | 0.88 | 100.00 | 407 | 405 | 2 |
| 57 | | 0.84 | 100.00 | 351 | 409 | 2 |
| | | | | | [MCH₃C00]⁻ | |
| 58 | | 0.79 | 95.26 | 351 | 409 | 2 |
| | | | | | [MCH₃C00]⁻ | |
| 59 | | 0.83 | 100.00 | 365 | 363 | 2 |
| 60 | 155.42^{(a)} | 2.09 | 100.00 | 348 | 346 | 1 |
| 61 | 185.16^{(a)} | 0.85 | 100.00 | 365 | 363 | 2 |
| 63 | | 1.88 | 96.00 | 323 | 321 | 1 |
| 64 | | 1.98 | 100.00 | 352 | 350 | 1 |
| 65 | | 0.99 | 96.09 | 327 | 325 | 2 |
| 66 | 128.58^{(a)} | 1.21 | 100.00 | 386 | 384 | 2 |
| 67 | | 1.11 | 98.40 | 322 | | 2 |
| 68 | 113.87^{(a)} | 1.04 | 99.02 | 308 | | 2 |
| 69 | | 0.76 | 1.05 | 332 | 330 | 2 |
| 70 | | 1.12 | 100.00 | 346 | 344 | 2 |
| 71 | 162.26^{(a)} | 2.11 | 100.00 | 334 | 332 | 7 |
| 72 | | 1.92 | 96.71 | 294 | | 4 |
| 73 | 109.5^{(b)} | 2.69 | 99.00 | 312 | | 6 |
| 74 | | 2.61 | 99.00 | 352 | | 6 |
| 75 | 199.77^{(a)} | 1.07 | 100.00 | 391 | 389 | 2 |
| 76 | 229.8^{(b)} | 2.37 | 98.00 | 372 | | 6 |
| 77 | 274.9^{(b)} | 1.65 | 99.00 | 348 | | 6 |
| 78 | | 1.49 | 100.00 | 313 | 311 | 1 |
| 79 | 186.62^{(a)} | 1.45 | 99.05 | 321 | 319 | 1 |
| 80 | 144.6^{(b)} | 1.79 | 99.00 | 313 | | 6 |
| 81 | | 1.51 | 100.00 | 345 | 343 | 1 |
| 82 | | 1.88 | 97.08 | 366 | | 1 |
| 83 | | 1.88 | 100.00 | 377 | 375 | 1 |
| 84 | | 1.45 | 100.00 | 320 | 318 | 1 |
| 85 | | 1.97 | 100.00 | 371 | 369 | 1 |
| 86 | 209.9^{(b)} | 2.21 | 96.00 | 349 | | 6 |
| 88 | | 2.18 | 96.00 | 377 | | 6 |
| 89 | 140.86^{(a)} | 1.82 | 100.00 | 348 | 346 | 1 |
| 90 | | 1.78 | 100.00 | 331 | 329 | 1 |
| 91 | | 1.99 | 98.32 | 382 | | 1 |
| 92 | | 1.62 | 100.00 | 343 | 341 | 1 |
| 93 | 172.70^{(a)} | 1.30 | 94.61 | 311 | 309 | 1 |
| 94 | | 1.58 | 100.00 | 343 | 341 | 1 |
| 95 | 131.72^{(a)} | 2.09 | 97.73 | 382 | 380 | |
| 96 | | 1.48 | 100.00 | 317 | 315 | 1 |
| 97 | | 1.88 | 95.83 | 356 | 354 | 1 |
| 98 | | 0.95 | 100.00 | 362 | | 2 |
| 99 | | 0.69 | 100.00 | 351 | 349 | 2 |
| 100 | 255.1^{(b)} | 2.52 | 99.00 | 348 | | 6 |
| 101 | | 1.86 | 98.90 | 326 | | 1 |
| 102 | | 0.77 | 100.00 | 365 | | 2 |
| 103 | | 1.43 | 98.34 | 358 | 356 | 1 |
| 104 | | 2.04 | 98.99 | 382 | 380 | 1 |
| 105a | | 2.19 | 94.08 | 348 | | 1 |
| 105b | | 2.18 | 100 | 348 | 346 | 1 |
| 106 | | 1.82 | 4.79 | 382 | 380 | 1 |
| 107 | | 1.63 | 94.44 | 309 | | 1 |
| 108 | | 1.7 | 1.95 | 327 | 325 | 1 |
| 109a | | 2.01 | 100 | 382 | 380 | 1 |
| 109b | | 2.01 | 99.06 | 382 | 380 | 1 |
| 110 | 155.45^{(b)} | 1.8 | 100 | 313 | | 1 |
| 111 | | 2.1 | 1.16 | 384 | 382 | 1 |
| 112 | | 1.82 | 100 | 359 | 357 | 1 |
| 113 | | 1.97 | 1.16 | 370 | 368 | 1 |
| 114 | | 1.85 | 100 | 395 | 393 | 1 |
| 115 | | 1.75 | 100 | 379 | 377 | 1 |
| 116 | | 2.08 | 100 | 428 | 430 | 1 |
| 117 | 173.7^{(b)} | 1.78 | 100 | 371 | 369 | 1 |
| 118 | | 2.2 | 100 | 410 | 408 | 1 |
| 119 | | 1.65 | 100 | 361 | | 1 |
| 120 | 239.1^{(b)} | 0.87 | 97.93 | 390 | 388 | 4 |
| 121 | 212.1^{(b)} | 1.83 | 100 | 377 | 375 | 1 |
| 122 | | 0.95 | 100 | 362 | | 4 |
| 123a | | 1.72 | 97.64 | 355 | 353 | 1 |
| 123b | | 1.72 | 93.1 | 355 | 353 | 1 |
| 124a | | 1.94 | 95.26 | 378 | 376 | 1 |
| 124b | | 1.9 | 97.92 | 378 | 376 | 1 |
| 125 | | 1.34 | 100 | 353 | | 1 |
| 126 | | 1.85 | 100 | 334 | 332 | 1 |
| 127 | | 0.91 | 98.42 | 355 | 353 | 4 |
| 128 | | 2.15 | 100 | 404 | 402 | 1 |
| 129 | | 2.12 | 98.56 | 404 | 402 | 1 |
| 130 | | 1.11 | 98 | 384 | 382 | 4 |
| 131 | | 0.99 | 97.9 | 385 | 383 | 4 |
| 132 | 181.8^{(b)} | 1.76 | 100 | 369 | 367 | 1 |
| 133 | | 0.76 | 96.21 | 334 | | 4 |
| 134 | | 0.81 | 100 | 353 | 351 | 4 |
| 135 | | 0.94 | 99.05 | 330 | | 4 |
| 136 | | 2.09 | 100 | 380 | 378 | 1 |
| 137 | | 1.55 | 100 | 323 | 321 | 1 |
| 138 | | 0.76 | 1.21 | 351 | | 4 |
| 139 | 151.7^{(b)} | 1.86 | 100 | 380 | | 1 |
| 140 | | 1.37 | 100 | 323 | 323 | 1 |
| 141 | | 1.58 | 100 | 351 | 349 | 1 |
| 142 | | 0.9 | 100 | 377 | | 4 |
| 143 | 238.4^{(b)} | 1.04 | 97.32 | 391 | 389 | 4 |
| 144 | | 0.62 | 100 | 297 | | 4 |
| 145a | | 1.59 | 100 | 328 | 326 | 1 |
| 145b | | 1.52 | 100 | 328 | 326 | 1 |
| 146 | | 1.04 | 100 | 409 | 407 | 4 |
| 147 | | 0.84 | 100 | 345 | 343 | 4 |
| 148 | 140.5^{(b)} | 1.04 | 100 | 362 | 360 | 4 |
| 149 | | 0.99 | 100 | 383 | 381 | 4 |
| 150 | 150.6^{(b)} | 1.04 | 100 | 350 | | 4 |
| 151 | 141.1^{(b)} | 1 | 100 | 354 | 352 | 4 |
| 152 | | 0.75 | 100 | 317 | 315 | 4 |
| 153 | | 1.94 | 97.64 | 352 | 350 | 1 |
| 154 | | 0.82 | 100 | 396 | | 4 |
| 155 | 161.4^{(b)} | 0.95 | 96.91 | 338 | | 4 |
| 156 | 181.9^{(b)} | 0.99 | 97.55 | 383 | | 4 |
| 157 | | 0.88 | 100 | 422 | 422 | 4 |
| 158 | | 0.99 | 100 | 391 | 389 | 4 |
| 159 | 140.8^{(b)} | 1.82 | 100 | 348 | 346 | 1 |
| 160 | | 1.58 | 97.99 | 337 | | 1 |
| 161 | | 2.24 | 100 | 404 | 402 | 1 |
| 162 | 148.9^{(b)} | 1.88 | 100 | 391 | 391 | 1 |
| 163 | | 2.05 | 97.5 | 354 | | 1 |
| 164 | | 1.2458 | 97 | 374 | 372 | 6 |
| 166 | | 1.1367 | 90 | 380 | 378 | 6 |
| 168 | | 0.9175 | 99 | 372 | 370 | 6 |
| 170 | | 1.0992 | 100 | 360 | 358 | 6 |
| 175 | | 1.0375 | 99 | 400 | 398 | 6 |
| 176 | | 1.3384 | 100 | 412 | 410 | 6 |
| 178 | | 1.1242 | 100 | 424 | 422 | 6 |
| 179 | | 1.2183 | 100 | 411 | 409 | 6 |
| 180 | | 1.2025 | 100 | 342 | 340 | 6 |
| 181 | | 0.9367 | 97 | 387 | 385 | 6 |
| 182 | | 1.1833 | 97 | 363 | 361 | 6 |
| 183 | | 1.12 | 100 | 377 | 375 | 6 |
| 184 | | 1.2308 | 100 | 376 | 374 | 6 |
| 185 | | 1.2092 | 100 | 413 | 411 | 6 |
| 186 | | 1.3492 | 99 | 366 | 364 | 6 |
| 187 | | 1.2283 | 100 | 428 | 426 | 6 |
| 188 | | 1.1242 | 97 | 411 | 409 | 6 |
| 189 | | 0.9542 | 100 | 398 | 396 | 6 |
| 190 | | 0.8383 | 100 | 362 | 360 | 6 |
| 191 | | 1.0483 | 98 | 358 | 356 | 6 |
| 192 | | 1.1992 | 96 | 364 | 362 | 6 |
| 193 | | 1.1658 | 99 | 336 | 334 | 6 |
| 194 | | 1.1517 | 97 | 361 | 359 | 6 |
| 195 | | 1.1183 | 99 | 370 | 368 | 6 |
| 197 | | 1.0683 | 96 | 361 | 359 | 6 |
| 200 | | 1.0525 | 97 | 374 | 372 | 6 |
| 201 | | 1.2458 | 97 | 362 | 360 | 6 |
| 202 | | 1.1358 | 97 | 345 | 343 | 6 |
| 208 | | 0.9175 | 99 | 404 | 402 | 6 |

### General Procedure for SFC-MS Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 3
Analytical SFC-MS Methods (Flow expressed in mL/min; column
temperature (T) in ° C.; run time in minutes; backpressure (BPR) in bars.

| | | | Flow | Run time |
|---|---|---|---|---|
| | | | [symbol] | [symbol] |
| Column | Mobile Phase | Gradient | T | BPR |
| Daicel Chiralpak ® | A: CO₂ | 10%-50% B in | 2.5 | 9.5 |
| IC-H column (3.0 | B: EtOH + 0.2% | 6 min, hold 3.5 | [symbol] | [symbol] |
| μm, 150 × 4.6 mm) | iPrNH₂ | min | 40 | 110 |

TABLE 4
Analytical SFC data—Rₜ means retention time (in minutes), [M + H]⁺
means the protonated mass of the compound, method refers to the
method used for (SFC)MS analysis of enantiomerically pure compounds.

| Co. No. | Rt (min) | UV Area % | [M + H]⁺ | [M − H]⁻ |
|---|---|---|---|---|
| 25b | 6.43 | 99.05 | 309 | 307 |
| 25a | 6.23 | 100 | 309 | 307 |

### Pharmacological Examples

### 1) OGA—Biochemical Assay

The assay is based on the inhibition of the hydrolysis of fluorescein mono-ß-D-N-Acetyl-Glucosamine (FM-GlcNAc) (Mariappa et al. 2015, Biochem J 470:255) by the recombinant human Meningioma Expressed Antigen 5 (MGEA5), also referred to as O-GlcNAcase (OGA). The hydrolysis FM-GlcNAc (Marker Gene technologies, cat #M1485) results in the formation of ß-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OnGene (cat #TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄ 2 H₂O (Sigma, #C0759) were dissolved in 1 L water to obtain a 200 mM solution. 19.2 g citric acid (Merck, #1.06580) was dissolved in 1 L water to obtain a 100 mM solution. pH of the sodium phosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at a 2 nM concentration and FM-GlcNAc at a 100 uM final concentration. Dilutions were prepared in assay buffer.

50 nl of a compound dissolved in DMSO was dispensed on Black Proxiplate™ 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000 rpm and incubate at room temperature for 6 h. To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

### 2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting 0-GlcNAcylated residues as previously described (Dorfmueller et al. 2010 Chemistry & biology, 17:1250). Inhibition of OGA will result in an increase of O-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, #D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100p of Assay Medium (Low Glucose medium is used to reduce basal levels of GlcNAcylation) (Park et al. 2014 The Journal of biological chemistry 289:13519). At the day of compound test medium from assay plates was removed and replenished with 90p1 of fresh Assay Medium. 10p of compounds at a 10 fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, #D8537). From next step onwards unless other stated assay volume was always 50p and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, #A16046), pH 7.5 (ICC buffer) before being permeabilized in same buffer for 10 minutes. Samples are subsequently blocked in ICC containing 5% goat serum (Sigma, #G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, #A-21042) and nuclei stained with Hoechst 33342 at a final concentration of 1 μg/ml in ICC (Lifetechnologies, #H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 5
Results in the biochemical and cellular assays.

| | Enzymatic | | Cellular | |
|---|---|---|---|---|
| Co. | hOGA; | Enzymatic | hOGA; | Cellular |
| No. | pIC₅₀ | Eₘₐₓ (%) | pEC₅₀ | Eₘₐₓ (%) |
| 1 | 6.82 | 102 | 6.15 | 56 |
| 2 | 7.04 | 99 | | |
| 3 | 6.03 | 92 | | |
| 4 | 6.03 | 93 | | |
| 5 | 7.95 | 101 | 7.17 | 67 |
| 6 | 8.16 | 102 | 7.3 | 89 |
| 7 | 7.98 | 103 | 6.67 | 84 |
| 8 | 8.11 | 102 | 6.96 | 87 |
| 9 | 8.42 | 105 | 7.52 | 85 |
| 11 | 7.06 | 100 | 6.54 | 80 |
| 12 | 7.31 | 99 | 6.25 | 64 |
| 13 | 8.49 | 105 | 7.55 | 87 |
| 14 | 8.54 | 103 | 6.74 | 80 |
| 15 | 7.18 | 101 | 6.25 | 71 |
| 16 | 7.83 | 101 | 7.49 | 85 |
| 17 | 6.94 | 102 | | |
| 18 | 7.46 | 102 | 6.24 | 62 |
| 19 | 7.8 | 102 | 7.08 | 94 |
| 20 | 7.4 | 102 | | |
| 21 | 6.61 | 96 | | |
| 22 | 6.54 | 99 | | |
| 23 | 8.84 | 102 | 8.53 | 97 |
| 24 | 8.8 | 102 | 8.47 | 91 |
| 25 | 8.32 | 104 | 7.42 | 89 |
| 25a | 8.23 | 102 | 7.3 | 92 |
| 25b | 8.34 | 101 | 7.63 | 90 |
| 26 | 6.72 | 100 | 6.32 | 64 |
| 27 | 8.65 | 104 | 8.12 | 112 |
| 28 | 5.66 | 83 | | |
| 29 | 5.79 | 88 | | |
| 30 | 5.85 | 93 | | |
| 31 | 6.52 | 99 | <6 | 27 |
| 32 | 6.63 | 98 | | |
| 33 | 6.51 | 99 | | |
| 34 | 6.75 | 99 | <6 | 36 |
| 35 | 6.83 | 100 | <6 | 20 |
| 36 | 6.56 | 100 | | |
| 37 | 6.6 | 101 | | |
| 38 | 6.94 | 100 | 6.25 | 63 |
| 39 | 7.27 | 102 | 6.16 | 56 |
| 40 | 7.09 | 102 | <6 | 30 |
| 41 | 7.61 | 101 | 7.02 | 71 |
| 42 | 7.46 | 100 | <6 | 49 |
| 43 | 7.62 | 99 | 6.17 | 50 |
| 44 | 7.45 | 100 | 6.52 | 69 |
| 45 | 7.98 | 103 | 7.78 | 87 |
| 46 | 7.75 | 99 | 6.6 | 67 |
| 47 | 7.93 | 101 | 7.32 | 95 |
| 48 | 7.97 | 102 | 7.51 | 86 |
| 49 | 7.93 | 103 | 7.1 | 63 |
| 50 | 7.9 | 102 | 6.52 | 78 |
| 51 | 7.95 | 102 | 6.93 | 76 |
| 52 | 8.02 | 103 | 7.57 | 72 |
| 53 | 8.73 | 105 | 7.69 | 82 |
| 54 | 8.69 | 104 | 7.68 | 82 |
| 55 | 8.77 | 103 | 7.65 | 104 |
| 56 | 8.57 | 101 | 8.29 | 107 |
| 57 | 8.34 | 102 | 7.96 | 107 |
| 58 | 8.51 | 101 | 7.14 | 88 |
| 59 | 8.36 | 101 | 7 | 88 |
| 60 | 8.44 | 102 | 7.26 | 86 |
| 61 | 8.43 | 103 | 8.23 | 92 |
| 62 | 8.27 | 101 | 8.03 | 95 |
| 63 | 8.54 | 103 | 7.91 | 93 |
| 64 | 8.4 | 102 | 7.61 | 80 |
| 65 | 8.27 | 104 | 6.92 | 69 |
| 66 | 8.42 | 103 | 6.73 | 84 |
| 67 | 9.05 | 104 | 7.79 | 99 |
| 68 | 8.42 | 104 | 7.24 | 87 |
| 69 | 8.47 | 102 | 7.26 | 82 |
| 70 | 8.77 | 103 | 7.43 | 82 |
| 71 | 8.65 | 101 | 7.32 | 78 |
| 72 | 8.25 | 100 | 7.27 | 78 |
| 73 | 8.57 | 101 | 7.27 | 93 |
| 74 | 7.54 | 100 | <6 | 40 |
| 75 | 8.55 | 102 | 8.22 | 85 |
| 76 | 8.53 | 101 | 7.63 | 100 |
| 77 | 8.32 | 99 | 8.13 | 83 |
| 78 | 8.49 | 103 | 8.1 | 96 |
| 79 | 6.86 | 101 | | |
| 80 | 7.54 | 100 | 6.43 | 73 |
| 81 | 8.02 | 102 | 6.99 | 74 |
| 82 | 8.71 | 103 | 7.67 | 87 |
| 83 | 6.04 | 96 | <6 | 8 |
| 84 | 5.67 | 86 | <6 | 8 |
| 85 | 9.01 | 104 | 9.15 | 94 |
| 86 | 7.66 | 101 | 6.51 | 71 |
| 87 | 8.32 | 102 | 7.89 | 87 |
| 88 | 8.39 | 103 | 7.97 | 95 |
| 90 | 7.31 | 102 | 6.44 | 62 |
| 91 | 8 | 102 | 7.1 | 73 |
| 92 | 7.38 | 103 | 6.82 | 75 |
| 93 | 7.44 | 101 | | |
| 94 | 6.86 | 99 | | |
| 95 | 7.61 | 104 | 6.28 | 63 |
| 96 | 7.1 | 102 | 6.29 | 60 |
| 97 | 7.9 | 101 | 6.46 | 78 |
| 98 | 6.54 | 98 | | |
| 99 | 7.75 | 102 | 7.04 | 81 |
| 100 | 8.25 | 104 | 7.15 | 81 |
| 101 | 8.49 | 103 | 7.24 | 83 |
| 102 | 6.64 | 99 | | |
| 103 | 6.12 | 99.735 | | |
| 104 | 6.14 | 96.86 | | |
| 105a | 7.51 | 102.725 | 6.07 | 49.31755 |
| 105b | 6.49 | 97.73 | | |
| 106 | 6.64 | 103.42 | | |
| 107 | 7.2 | 102.49 | 6.41 | 72.4444 |
| 108 | 7.32 | 101.5 | | |
| 109a | 7.67 | 103.365 | 6.35 | 70.00575 |
| 109b | 7.4 | 103.505 | 6.21 | 62.63105 |
| 110 | 7.54 | 100.33 | 6.44 | 48.0484 |
| 111 | 7.62 | 102.06 | | |
| 112 | 8.88 | 101.71 | ~8.74 | 92.2672 |
| 113 | 8.9 | 106.41 | 8.1 | 93.0309 |
| 114 | 7.03 | 100.065 | 6.06 | 49.5545 |
| 115 | 7.2 | 99.59 | ~6.05 | 52.30405 |
| 116 | 7.98 | 103.56 | 7.26 | 61.80555 |
| 117 | 8.26 | 101.215 | 7.35 | 89.60805 |
| 118 | 7.89 | 102.49 | 7.13 | 71.65095 |
| 119 | 8 | 104.835 | 6.99 | 80.29385 |
| 120 | 6.77 | 99.16 | | |
| 121 | 7.25 | 102.525 | | |
| 122 | 7.72 | 103.22 | 6.58 | 84.0559 |
| 123a | 7.56 | 101.785 | 6.36 | 63.77535 |
| 123b | 7.53 | 99.245 | 7 | 71.089 |
| 124a | 7.88 | 102.79 | 7.66 | 74.67885 |
| 124b | 8.04 | 101.97 | 7.56 | 76.70975 |
| 125 | 7.91 | 103.375 | 7.36 | 74.82105 |
| 126 | 8.67 | 100.075 | 7.98 | 87.0992 |
| 127 | 8.75 | 103.315 | 7.94 | 76.7132 |
| 128 | 8.58 | 100.75 | 7.89 | 90.31005 |
| 129 | 8.59 | 100.47 | 8.13 | 104.0232 |
| 130 | 8.6 | 102.195 | 8.5 | 93.6924 |
| 131 | 8.36 | 102.16 | 7.81 | 78.6533 |
| 132 | 7.94 | 99.715 | 6.96 | 82.2019 |
| 133 | 8.16 | 100.71 | 7.06 | 76.10895 |
| 134 | 8.28 | 99.895 | 7.09 | 77.501 |
| 135 | 8.64 | 98.82 | 8.03 | 82.2068 |
| 136 | 7.64 | 101.11 | 6.62 | 74.9413 |
| 137 | 7.14 | 99.125 | 6.29 | 60.4354 |
| 138 | 8.07 | 102.94 | 7.13 | 66.8173 |
| 139 | 7.8 | 104.24 | 6.4 | 69.6531 |
| 140 | 7.08 | 103.41 | 6.22 | 63.89145 |
| 141 | 7.76 | 102.875 | 6.97 | 74.70745 |
| 142 | 6.56 | 99.25 | | |
| 143 | 7.41 | 100.34 | | |
| 144 | 7.25 | 100.36 | 6.74 | 79.03785 |
| 145a | 7.79 | 100.115 | 7.25 | 81.17225 |
| 145b | 8.46 | 101.69 | 7.57 | 85.6778 |
| 146 | 7.56 | 100.855 | 7.34 | 76.3091 |
| 147 | 8.81 | 98.635 | ~8.35 | 90.89435 |
| 148 | 8.89 | 102.07 | ~8.32 | 87.4542 |
| 149 | 8.58 | 100.825 | 7.97 | 96.36615 |
| 150 | 8.57 | 101.215 | 8.27 | 82.12015 |
| 151 | 8.61 | 100.315 | 7.99 | 101.6189 |
| 152 | 8.5 | 101.115 | 7.52 | 97.3957 |
| 153 | 8.52 | 102.395 | 7.69 | 74.13665 |
| 154 | 8.67 | 100.825 | 8.46 | 86.15435 |
| 155 | 8 | 100.975 | 7.3 | 73.3467 |
| 156 | 8.07 | 101.715 | 7.34 | 70.885 |
| 157 | 7.99 | 99.265 | 7.56 | 95.2079 |
| 158 | 8.58 | 101.14 | 7.2 | 70.9797 |
| 159 | 8.87 | 104.285 | 7.69 | 83.97355 |
| 160 | 8.67 | 102.645 | 8.09 | 95.965 |
| 161 | 8.58 | 103.575 | 8.17 | 88.812 |
| 162 | 8.59 | 103.665 | 7.99 | 85.80495 |
| 163 | 8.63 | 103.52 | 8.38 | 90.2664 |
| 164 | 7.43 | 100.645 | | |
| 165 | 7.42 | 101.05 | | |
| 166 | 7.32 | 101.145 | 6 | 44.09655 |
| 167 | 7.77 | 101.535 | ~6.65 | 81.9417 |
| 168 | 6.25 | 98.08 | | |
| 169 | 7.92 | 104.445 | 6.26 | 67.5494 |
| 170 | 7.56 | 100.34 | 7.08 | 78.57035 |
| 171 | 8.22 | 102.85 | 7.41 | 73.7683 |
| 172 | 8.47 | 102.06 | 7.15 | 80.8014 |
| 173 | 7.63 | 100.725 | 6.8 | 88.52535 |
| 174 | 7.89 | 99.33 | 7.03 | 87.0352 |
| 175 | 8.2 | 100.495 | 7.43 | 80.08945 |
| 176 | 8.81 | 102.905 | 8.26 | 101.3075 |
| 177 | | | | |
| 178 | 8.95 | 101.215 | 8.53 | 102.5392 |
| 179 | 8.69 | 101.9 | 7.69 | 80.8956 |
| 180 | 7.71 | 100.025 | 7.09 | 84.4394 |
| 181 | 6.1 | 92.68 | | |
| 182 | 6.51 | 97.805 | | |
| 183 | 6.3 | 95.29 | <6 | 13.87005 |
| 184 | 7.1 | 100.845 | 6.04 | 46.5431 |
| 185 | 8.65 | 101.665 | 8.01 | 99.77105 |
| 186 | 8.37 | 101.65 | 7.61 | 85.8858 |
| 187 | 8.75 | 101.04 | 8.06 | 96.35835 |
| 188 | 8.79 | 100.39 | ~8.36 | 89.05235 |
| 189 | 8.98 | 100.42 | 8.5 | 101.3393 |
| 190 | 6.25 | 96.315 | | |
| 191 | 6.64 | 98.825 | | |
| 192 | 6.74 | 98.315 | | |
| 193 | 8.2 | 101.64 | 6.95 | 89.69315 |
| 194 | 8.11 | 103.785 | 7.1 | 67.8969 |
| 195 | 8.87 | 101.94 | 7.6 | 95.2942 |
| 196 | 8.82 | 104.05 | 8.07 | 91.65555 |
| 197 | 8.31 | 104.36 | 7.25 | 86.2826 |
| 199 | | | | |
| 200 | 7.63 | 103.085 | 6.29 | 70.12265 |
| 201 | 6.19 | 95.09 | | |
| 202 | 8.51 | 101.43 | 8.44 | 88.08685 |
| 203 | 7.13 | 99.545 | 6.66 | 73.6534 |
| 204 | 7.24 | 101.135 | 6.57 | 76.0909 |
| 205 | 8.52 | 98.185 | 8.02 | 88.61055 |
| 206 | 7.29 | 101.44 | 6.18 | 63.6645 |
| 207 | 7.13 | 101.28 | 6.29 | 63.19185 |
| 208 | 7.99 | 100.63 | 7.14 | 90.14845 |
| 209 | | | | |

1. A compound of Formula (I) 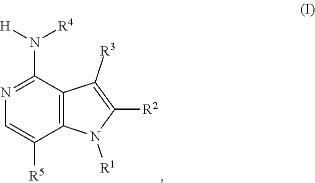
or a tautomer or a stereoisomeric form thereof, wherein
R¹ is selected from the group consisting of C₁₋₆alkyl optionally substituted with one or more substituents each independently selected from the group consisting of halo, —CN, —OC₁₋₃alkyl, —OH, —SO₂NR^{5a}R^{6a}, and C₃₋₆cycloalkyl optionally substituted with one or more independently selected halo substituents; C₁₋₆alkyl substituted with oxetanyl; and C₁₋₆alkyl wherein two geminal hydrogens are replaced by oxetanylidene; wherein R^{5a} and R^{6a} are each independently selected from the group consisting of hydrogen and C₁₋₃alkyl; with the proviso that a —OC₁₋₃alkyl or —OH substituent, when present, is at least two carbon atoms away from the nitrogen atom of the 1H-pyrrolo[3.2-c]pyridine;
R², R³ and R⁵ are each independently selected from the group consisting of hydrogen, halo and C₁₋₃alkyl;
R⁴ is a monovalent radical selected from the group consisting of (a), (b), (c), and (d): 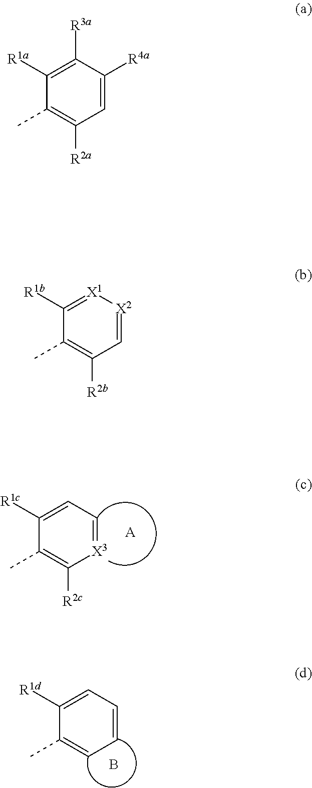
wherein
R^{1a}, R^{2a}, R^{1b}, and R^{2b} are each independently selected from the group consisting of halo, C₁₋₃alkyl, monohaloC₁₋₃alkyl, polyhaloC₁₋₃alkyl, C₁₋₃alkyloxy, monohaloC₁₋₃alkyloxy, polyhaloC₁₋₃alkyloxy, and C₃₋₆cycloalkyl;
R^{3a} is selected from the group consisting of hydrogen, halo, —C(O)—OC₁₋₃alkyl, —C(O)—NR′R″, and —N(R′″)—C(O)—C₁₋₃alkyl;
R^{4a} is selected from the group consisting of hydrogen, halo, —CN, C₁₋₃alkyl, monohaloC₁₋₃alkyl, polyhaloC₁₋₃alkyl, —C(O)—OC₁₋₃alkyl, —C(O)—NR′R″, —N(R′″)—C(O)—C₁₋₃alkyl, and Het;
with the proviso that R^{3a} and R^{4a} are not simultaneously —C(O)—OC₁₋₃alkyl, —C(O)—NR′R″, or —N(R′″)—C(O)—C₁₋₃alkyl;
R′ and R″ are each independently selected from the group consisting of hydrogen and C₁₋₃alkyl; or R′ and R″ together with the nitrogen atom to which they are attached form a heterocyclyl ring selected from the group consisting of azetidinyl, pyrrolidinyl, piperidinyl, piperazinyl and morpholinyl;
R′″ is selected from the group consisting of hydrogen and C₁₋₃alkyl;
Het is pyrazolyl or imidazolyl, optionally substituted with one or more independently selected C₁₋₃alkyl substituents;
X¹ and X² are each independently selected from N and CH, with the proviso that at least one of X¹ or X² is N;
R^{1c}, R^{2c}, and R^{1d} are each independently selected from the group consisting of halo, C₁₋₃alkyl, monohaloC₁₋₃alkyl, polyhaloC₁₋₃alkyl, C₁₋₃alkyloxy, monohaloC₁₋₃alkyloxy, polyhaloC₁₋₃alkyloxy, and C₃₋₆cycloalkyl;
X³ represents CH or N;
and each of the rings represented by 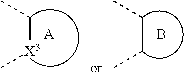
form
(i) a 5- or 6-membered unsaturated heterocycle having one, two or three heteroatoms each independently selected from nitrogen and oxygen, and which is optionally substituted with one or more substituents, each independently selected from halo, C₁₋₃alkyl and oxo; or
(ii) an aromatic heterocycle having one, two or three heteroatoms each independently selected from nitrogen, oxygen, and sulfur, and which is optionally substituted with one or more substituents, each independently selected from halo, —CN, C₁₋₃alkyl, monohaloC₁₋₃alkyl, and polyhaloC₁₋₃alkyl;
or a pharmaceutically acceptable addition salt or a solvate thereof.

2. The compound according to claim 1, wherein
R¹ is selected from the group consisting of C₁₋₆alkyl optionally substituted with one, two or three substituents each independently selected from the group consisting of halo, —CN, —OC₁₋₃alkyl, —OH, —SO₂NR^{5a}R^{6a}, and C₃₋₆cycloalkyl optionally substituted with one, two or three independently selected halo substituents; C₁₋₆alkyl substituted with oxetanyl; and C₁₋₆alkyl wherein two geminal hydrogens are replaced by oxetanylidene; wherein R^{5a} and R^{6a} are each independently selected from the group consisting of hydrogen and C₁₋₃alkyl; with the proviso that a —OC₁₋₃alkyl or —OH substituent, when present, is at least two carbon atoms away from the nitrogen atom of the 1H-pyrrolo[3.2-c]pyridine;
R², R³ and R⁵ are each independently selected from the group consisting of hydrogen, halo and C₁₋₃alkyl;
R⁴ is a monovalent radical selected from the group consisting of (a), (b), (c), and (d), wherein R^{1a}, R^{2a}, R^{1b}, and R^{2b} are each independently selected from the group consisting of halo, C₁₋₃alkyl, monohaloC₁₋₃alkyl, polyhaloC₁₋₃alkyl, and C₃₋₆cycloalkyl;
R^{3a} is selected from the group consisting of hydrogen, halo, —C(O)—NR′R″, and —N(R′″)—C(O)—C₁₋₃alkyl;
R^{4a} is selected from the group consisting of hydrogen, halo, C₁₋₃alkyl, monohaloC₁₋₃alkyl, polyhaloC₁₋₃alkyl, —C(O)—OC₁₋₃alkyl, —C(O)—NR′R″, —N(R′″)—C(O)—C₁₋₃alkyl, and Het; with the proviso that R^{3a} and R^{4a} are not simultaneously —C(O)—OC₁₋₃alkyl, —C(O)—NR′R″, or —N(R′″)—C(O)—C₁₋₃alkyl;
R′ and R″ are each independently selected from the group consisting of hydrogen and C₁₋₃alkyl; or R′ and R″ together with the nitrogen atom to which they are attached form a heterocyclyl ring selected from the group consisting of azetidinyl, pyrrolidinyl, piperidinyl, piperazinyl and morpholinyl;
R′″ is selected from the group consisting of hydrogen and C₁₋₃alkyl;
Het is pyrazolyl or imidazolyl, optionally substituted with one or more independently selected C₁₋₃alkyl substituents;
X¹ and X² are each independently selected from N and CH, with the proviso that at least one of X¹ or X² is N;
R^{1c}, R^{2c}, and R^{1d} each independently represent halo or C₁₋₃alkyl;
X³ represents CH or N;
and each of the rings represented by 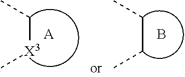
form
(i) a 5- or 6-membered unsaturated heterocycle having one, two or three heteroatoms each independently selected from nitrogen and oxygen, and which is optionally substituted with one or two substituents, each independently selected from halo, C₁₋₃alkyl and oxo; or
(ii) an aromatic heterocycle having one, two or three heteroatoms each independently selected from nitrogen and oxygen, and which is optionally substituted with one or two substituents, each independently selected from C₁₋₃alkyl.

3. The compound according to claim 1, wherein R¹ is selected from the group consisting of C₁₋₆alkyl optionally substituted with one, two or three substituents each independently selected from the group consisting of halo, and C₃₋₆cycloalkyl optionally substituted with one, two or three independently selected halo substituents; C₁₋₆alkyl substituted with oxetanyl; and C₁₋₆alkyl wherein two geminal hydrogens are replaced by oxetanylidene;
R², R³ and R⁵ are each independently selected from the group consisting of hydrogen, halo and C₁₋₃alkyl;
R⁴ is a monovalent radical selected from the group consisting of (a), (b), (c), and (d), wherein R^{1a}, R^{2a}, R^{1b}, and R^{2b} are each independently selected from the group consisting of halo, C₁₋₃alkyl, monohaloC₁₋₃alkyl, polyhaloC₁₋₃alkyl, and C₃₋₆cycloalkyl;
R^{3a} is selected from the group consisting of hydrogen, halo, and —C(O)—NR′R″;
R^{4a} is selected from the group consisting of hydrogen, halo, C₁₋₃alkyl, monohaloC₁₋₃alkyl, polyhaloC₁₋₃alkyl, —C(O)—OC₁₋₃alkyl, —C(O)—NR′R″, —N(R′″)—C(O)—C₁₋₃alkyl, and Het; with the proviso that R^{3a} and R^{4a} are not simultaneously —C(O)—OC₁₋₃alkyl, —C(O)—NR′R″, or —N(R′″)—C(O)—C₁₋₃alkyl;
R′ and R″ are each independently selected from the group consisting of hydrogen and C₁₋₃alkyl; or R′ and R″ together with the nitrogen atom to which they are attached form a heterocyclyl ring selected from the group consisting of pyrrolidinyl, and morpholinyl;
R′″ is selected from the group consisting of hydrogen and C₁₋₃alkyl;
Het is pyrazolyl or imidazolyl, optionally substituted with one or more independently selected C₁₋₃alkyl substituents;
X¹ and X² are each independently selected from N and CH, with the proviso that at least one of X¹ or X² is N;
R^{1c}, R^{2c}, and R^{1d} each independently represent halo or C₁₋₃alkyl;
X³ represents CH or N;
and each of the rings represented by 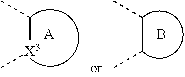
form
(i) a 5- or 6-membered unsaturated heterocycle having one, two or three heteroatoms each independently selected from nitrogen and oxygen, and which is optionally substituted with one or two substituents, each independently selected from halo, C₁₋₃alkyl and oxo; or
(ii) an aromatic heterocycle having one, two or three heteroatoms each independently selected from nitrogen and oxygen, and which is optionally substituted with one or two substituents, each independently selected from C₁₋₃alkyl.

4. The compound according to claim 1, wherein R¹ is C₁₋₆alkyl optionally substituted with one, two or three substituents each independently selected from the group consisting of halo, and C₃₋₆cycloalkyl optionally substituted with one, two or three independently selected halo substituents or R¹ is C₁₋₆alkyl substituted with oxetanyl or C₁₋₆alkyl wherein two geminal hydrogens are replaced by oxetanylidene.

5. The compound according to claim 1, wherein R² and R³ are each independently selected from hydrogen and fluoro.

6. The compound according to claim 1, wherein R⁵ is hydrogen, fluoro or methyl.

7. A pharmaceutical composition comprising a prophylactically or a therapeutically effective amount of a compound according to claim 1 and a pharmaceutically acceptable carrier.

8. A process for preparing a pharmaceutical composition comprising mixing a pharmaceutically acceptable carrier with a prophylactically or a therapeutically effective amount of a compound according to claim 1.

9. (canceled)

10. (canceled)

11. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound according to claim 1.

12. (canceled)
